SEQUENCE LISTING (25 sequences)

SEQ ID NO: 1 - protein, 219 aa, Artificial Sequence
  humanized immunoglobulin
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 447 aa, Artificial Sequence
  humanized immunoglobulin
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 24 bp, Artificial Sequence
  forward primer
tgtggagacaggggcttttatttc

SEQ ID NO: 4 - DNA, 24 bp, Artificial Sequence
  forward primer
tgtggagacaggggcttttatttc

SEQ ID NO: 5 - DNA, 24 bp, Artificial Sequence
  forward primer
tgtggagacaggggcttttatttc

SEQ ID NO: 6 - DNA, 24 bp, Artificial Sequence
  forward primer
tgtggagacaggggcttttatttc

SEQ ID NO: 7 - DNA, 24 bp, Artificial Sequence
  forward primer
tgtggagacaggggcttttatttc

SEQ ID NO: 8 - DNA, 20 bp, Artificial Sequence
  reverse primer
gcagcactcatccacgatgc

SEQ ID NO: 9 - DNA, 20 bp, Artificial Sequence
  reverse primer
gcagcactcatccacgatgc

SEQ ID NO: 10 - DNA, 20 bp, Artificial Sequence
  reverse primer
gcagcactcatccacgatgc

SEQ ID NO: 11 - DNA, 20 bp, Artificial Sequence
  reverse primer
gcagcactcatccacgatgc

SEQ ID NO: 12 - DNA, 20 bp, Artificial Sequence
  reverse primer
gcagcactcatccacgatgc

SEQ ID NO: 13 - DNA, 7370 bp, Homo sapiens
ttttgtagataaatgtgaggattttctctaaatccctcttctgtttgctaaatctcactgtcactgctaaattcagagcagatagagcctgcgcaatggaataaagtcctcaaaattgaaatgtgacattgctctcaacatctcccatctctctggatttctttttgcttcattattcctgctaaccaattcattttcagactttgtacttcagaagcaatgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacgcaagtagagggagtgcaggaaacaagaactacaggatgtaggaagaccctcctgaggagtgaagagtgacatgccaccgcaggatcctttgctctgcacgagttacctgttaaactttggaacacctaccaaaaaataagtttgataacatttaaaagatgggcgtttcccccaatgaaatacacaagtaaacattccaacattgtctttaggagtgatttgcaccttgcaaaaatggtcctggagttggtagattgctgttgatcttttatcaataatgttctatagaaaagaaaaaaaaaatatatatatatatatatcttagtccctgcctctcaagagccacaaatgcatgggtgttgtatagatccagttgcactaaattcctctctgaatcttggctgctggagccattcattcagcaaccttgtctaagtggtttatgaattgtttccttatttgcacttctttctacacaactcgggctgtttgttttacagtgtctgataatcttgttagtctatacccaccacctcccttcataacctttatatttgccgaatttggcctcctcaaaagcagcagcaagtcgtcaagaagcacaccaattctaacccacaagattccatctgtggcatttgtaccaaatataagttggatgcattttattttagacacaaagctttatttttccacatcatgcttacaaaaaagaataatgcaaatagttgcaactttgaggccaatcatttttaggcatatgttttaaacatagaaagtttcttcaactcaaaagagttccttcaaatgatgagttaatgtgcaacctaattagtaactttcctctttttattttttccatatagagcactatgtaaatttagcatatcaattatacaggatatatcaaacagtatgtaaaactctgttttttagtataatggtgctattttgtagtttgttatatgaaagagtctggccaaaacggtaatacgtgaaagcaaaacaataggggaagcctggagccaaagatgacacaaggggaagggtactgaaaacaccatccatttgggaaagaaggcaaagtccccccagttatgccttccaagaggaacttcagacacaaaagtccactgatgcaaattggactggcgagtccagagaggaaactgtggaatggaaaaagcagaaggctaggaattttagcagtcctggtttctttttctcatggaagaaatgaacatctgccagctgtgtcatggactcaccactgtgtgaccttgggcaagtcacttcacctctctgtgcctcagtttcctcatctgcaaaatgggggcaatatgtcatctacctacctcaaaggggtggtataaggtttaaaaagataaagattcagattttttttaccctgggttgctgtaagggtgcaacatcagggcgcttgagttgctgagatgcaaggaattctataaataacccattcatagcatagctagagattggtgaattgaatgctcctgacatctcagttcttgtcagtgaagctatccaaataactggccaactagttgttaaaagctaacagctcaatctcttaaaacacttttcaaaatatgtgggaagcatttgattttcaatttgattttgaattctgcatttggttttatgaatacaaagataagtgaaaagagagaaaggaaaagaaaaaggagaaaaacaaagagatttctaccagtgaaaggggaattaattactctttgttagcactcactgactcttctatgcagttactacatatctagtaaaacctcgtttaatactataaataatattctattcattttgaaaaacacaatgattccttcttttctaggcaatataaggaaagtgatccaaaatttgaaatattaaaataatatctaataaaaagtcacaaagttatcttctttaacaaactttactcttattcttagctgtatatacatttttttaaaagtttgttaaaatatgcttgactagagtttccagttgaaaggcaaaaacttccatcacaacaagaaatttcccatgcctgctcagaagggtagcccctagctctctgtgaatgtgttttatccattcaactgaaaattggtatcaagaaagtccactggttagtgtactagtccatcatagcctagaaaatgatccctatctgcagatcaagattttctcattagaacaatgaattatccagcattcagatctttctagtcaccttagaactttttggttaaaagtacccaggcttgattatttcatgcaaattctatattttacattcttggaaagtctatatgaaaaacaaaaataacatcttcagtttttctcccactgggtcacctcaaggatcagaggccaggaaaaaaaaaaaaaagactccctggatctctgaatatatgcaaaaagaaggccccatttagtggagccagcaatcctgttcagtcaacaagtattttaactctcagtccaacattatttgaattgagcacctcaagcatgcttagcaatgttctaatcactatggacagatgtaaaagaaactatacatcatttttgccctctgcctgttttccagacatacaggttctgtggaataagatactggactcctcttcccaagatggcacttctttttatttcttgtccccagtgtgtaccttttaaaattattccctctcaacaaaactttataggcagtcttctgcagacttaacgtgttttctgtcatagttagatgtgataattctaagagtgtctatgacttatttccttcacttaattctatccacagtcaaaaatcccccaaggaggaaagctgaaagatgcactgccatattatctttcttaactttttccaacacataatcctctccaactggattataaataaattgaaaataactcattataccaattcactattttattttttaatgaattaaaactagaaaacaaattgatgcaaaccctggaagtcagttgattactatatactacagcagaatgactcagatttcatagaaaggagcaaccaaaatgtcacaacccaaaactttacaagctttgcttcagaattagattgctttataattcttgaatgaggcaatttcaagatatttgtaaaagaacagtaaacattggtaagaatgagctttcaactcataggcttatttccaatttaattgaccatactggatacttaggtcaaatttctgttctctcttccccaaataatattaaagtattatttgaactttttaagatgaggcagttcccctgaaaaagttaatgcagctctccatcagaatccactcttctagggatatgaaaatctcttaacacccaccctacatacacagacacacacacacacacacacacacacacacacacacacattcaccctaaggatccaatggaatactgaaaagaaatcacttccttgaaaattttattaaaaaacaaacaaacaaacaaaaagcctgtccacccttgagaatccttcctctccttggaacgtcaatgtttgtgtagatgaaaccatctcatgctctgtggctccagggtttctgttactattttatgcacttgggagaaggcttagaataaaagatgtagcacattttgctttcccatttattgtttggccagctatgccaatgtggtgctattgtttctttaagaaagtacttgactaaaaaaaaaagaaaaaaagaaaaaaaagaaagcatagacatatttttttaaagtataaaaacaacaattctatagatagatggcttaataaaatagcattaggtctatctagccaccaccacctttcaactttttatcactcacaagtagtgtactgttcaccaaattgtgaatttgggggtgcaggggcaggagttggaaattttttaaagttagaaggctccattgttttgttggctctcaaacttagcaaaattagcaatatattatccaatcttctgaacttgatcaagagcatggagaataaacgcgggaaaaaagatcttataggcaaatagaagaatttaaaagataagtaagttccttattgatttttgtgcactctgctctaaaacagatattcagcaagtggagaaaataagaacaaagagaaaaaatacatagatttacctgcaaaaaatagcttctgccaaatcccccttgggtattctttggcatttactggtttatagaagacattctcccttcacccagacatctcaaagagcagtagctctcatgaaaagcaatcactgatctcatttgggaaatgttggaaagtatttccttatgagatgggggttatctactgataaagaaagaatttatgagaaattgttgaaagagatggctaacaatctgtgaagattttttgtttcttgtttttgttttttttttttttttactttatacagtctttatgaatttcttaatgttcaaaatgacttggttcttttcttctttttttatatcagaatgaggaataataagttaaacccacatagactctttaaaactataggctagatagaaatgtatgtttgacttgttgaagctataatcagactatttaaaatgttttgctatttttaatcttaaaagattgtgctaatttattagagcagaacctgtttggctctcctcagaagaaagaatctttccattcaaatcacatggctttccaccaatattttcaaaagataaatctgatttatgcaatggcatcatttattttaaaacagaagaattgtgaaagtttatgcccctcccttgcaaagaccataaagtccagatctggtaggggggcaacaacaaaaggaaaatgttgttgattcttggttttggattttgttttgttttcaatgctagtgtttaatcctgtagtacatatttgcttattgctattttaatattttataagaccttcctgttaggtattagaaagtgatacatagatatcttttttgtgtaatttctatttaaaaaagagagaagactgtcagaagctttaagtgcatatggtacaggataaagatatcaatttaaataaccaattcctatctggaacaatgcttttgttttttaaagaaacctctcacagataagacagaggcccaggggatttttgaagctgtctttattctgcccccatcccaacccagcccttattattttagtatctgcctcagaattttatagagggctgaccaagctgaaactctagaattaaaggaacctcactgaaaacatatatttcacgtgttccctctttttttttttcctttttgtgagatggggtctcgcactgtcccccaggctggagtgcagtggcatgatctcggctcactgcaacctccacctcctgggtttaagcgattctcctgcctcagcctcctgagtagctgggattacaggcacccaccactatgcccggctaattttttggatttttaatagagacggggttttaccatgttggccaggttggtctcaaactcctgaccttgtgatttgcccgcctcagcctcccaaattgctgggattacaggcatgagccaccacaccctgcccatgtgttccctcttaatgtatgattacatggatcttaaacatgatccttctctcctcattcttcaactatctttgatggggtctttcaaggggaaaaaaatccaagcttttttaaagtaaaaaaaaaaaaagagaggacacaaaaccaaatgttactgctcaactgaaatatgagttaagatggagacagagtttctcctaataaccggagctgaattacctttcactttcaaaaacatgaccttccacaatccttagaatctgcctttttttatattactgaggcctaaaagtaaacattactcattttattttgcccaaaatgcactgatgtaaagtaggaaaaataaaaacagagctctaaaatccctttcaagccacccattgaccccactcaccaactcatagcaaagtcacttctgttaatcccttaatctgattttgtttggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtcttagttgaaaagcatattttttattaaattaattctgattgtatttgaaattattattcaattcacttatggcagaggaatatcaatcctaatgacttctaaaaatgtaactaattgaatcattatcttacatttactgtttaataagcatattttgaaaatgtatggctagagtgtcataataaaatggtatatctttctttagtaattacattaaaattagtcatgtttgattaattagttc

SEQ ID NO: 14 - DNA, 1621 bp, Homo sapiens
gcggcaggtctctgcgcagcccagcccgccggtccacgccgcgcaccgctccgagggccagcgccacccgctccgcagccggcaccatgcgcgagatcgtgcacatccaggcgggccagtgcggcaaccagatcggcgccaagttttgggaggtcatcagcgatgagcatgggatcgaccccacaggcagttaccatggagacagtgacttgcagctggagagaatcaacgtgtactacaatgaggctgctggtaacaaatatgtacctcgggccatcctggtggatctggagcctggcaccatggactctgtcaggtctggacccttcggccagatcttcagaccagacaacttcgtgttcggccagagtggagccgggaataactgggccaagggccactacacagagggagccgagctggtcgactcggtcctggatgtggtgaggaaggagtcagagagctgtgactgtctccagggcttccagctgacccactctctggggggcggcacggggtccgggatgggcaccctgctcatcagcaagatccgggaagagtacccagaccgcatcatgaacaccttcagcgtcatgccctcacccaaggtgtcagacacggtggtggagccctacaacgccaccctctctgtccaccagctggtggaaaacacagatgaaacctactccattgataacgaggccctgtatgacatctgcttccgcaccctgaagctgaccacccccacctacggggacctcaaccacctggtgtcggccaccatgagcggggtcaccacctgcctgcgcttcccgggccagctgaacgcagacctgcgcaagctggcggtgaacatggtgcccttccctcgcctgcacttcttcatgcccggcttcgcgcccctgaccagccggggcagccagcagtaccgggcgctcacggtgcccgagctcacccagcagatgttcgactccaagaacatgatggccgcctgcgacccgcgccacggccgctacctgacggtggctgccatcttccggggccgcatgtccatgaaggaggtggacgagcagatgctcaacgtgcagaacaagaacagcagctacttcgtggagtggatccccaacaacgtgaagacggccgtgtgcgacatcccgccccgcggcctgaagatgtcggccaccttcatcggcaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgacgaacaaggggagttcgaggaggaggagggcgaggacgaggcttaaaaacttctcagatcaatcgtgcatccttagtgaacttctgttgtcctcaagcatggtctttctacttgtaaactatggtgctcagttttgcctctgttagaaattcacactgttgatgtaatgatgtggaactcctctaaaaattacagtattgtctgtgaaggtatctatactaataaaaaagcatgtgtagaaaa

SEQ ID NO: 15 - DNA, 5410 bp, Homo sapiens
agtgggccgccatgttgtcggagtgaaaggtaagggggagcgagagcgccagagagagaagatcggggggctgaaatccatcttcatcctaccgctccgcccgtgttggtggaatgagcgttgcatgtgtcttgaagagaaaagcagtgctttggcaggactctttcagcccccacctgaaacatcaccctcaagaaccagctaatcccaacatgcctgttgttttgacatctggaacagggtcgcaagcgcagccacaaccagctgcaaatcaggctcttgcagctgggactcactccagccctgtcccaggatctataggagttgcaggccgttcccaggacgacgctatggtggactacttctttcagaggcagcatggtgagcagcttgggggaggaggaagtggaggaggcggctataataatagcaaacatcgatggcctactggggataacattcatgcagaacatcaggtgcgttccatggatgaactgaatcatgattttcaagcacttgctctggagggaagagcgatgggagagcagctcttgccaggtaaaaagttttgggaaacagatgaatccagcaaagatggaccaaaaggaatattcctgggtgatcaatggcgagacagtgcctggggaacatcagatcattcagtttcccagccaatcatggtgcagagaagacctggtcagagtttccatgtgaacagtgaggtcaattctgtactgtccccacgatcggagagtgggggactaggcgttagcatggtggagtatgtgttgagctcatccccgggcgattcctgtctaagaaaaggaggatttggcccaagggatgcagacagtgatgaaaacgacaaaggtgaaaagaagaacaagggtacgtttgatggagataagctaggagatttgaaggaggagggtgatgtgatggacaagaccaatggtttaccagtgcagaatgggattgatgcagacgtcaaagattttagccgtacccctggtaattgccagaactctgctaatgaagtggatcttctgggtccaaaccagaatggttctgagggcttagcccagctgaccagcaccaatggtgccaagcctgtggaggatttctccaacatggagtcccagagtgtccccttggaccccatggaacatgtgggcatggagcctcttcagtttgattattcaggcacgcaggtacctgtggactcagcagcagcaactgtgggactttttgactacaattctcaacaacagctgttccaaagacctaatgcgcttgctgtccagcagttgacagctgctcagcagcagcagtatgcactggcagctgctcatcagccgcacatcggtttagctcccgctgcgtttgtccccaatccatacatcatcagcgctgctcccccagggacggacccctacacagctggattggctgcagcagcgacactaggcccagctgtggtccctcaccagtattatggagttactccctggggagtctaccctgccagtcttttccagcagcaagctgccgctgccgctgcagcaactaattcagctaatcaacagaccaccccacaggctcagcaaggacagcagcaggttctccgtggaggagccagccaacgtcctttgaccccaaaccagaaccagcagggacagcaaacggatccccttgtggcagctgcagcagtgaattctgcccttgcatttggacaaggtctggcagcaggcatgccaggttatccggtgttggctcctgctgcttactatgaccaaactggtgcccttgtagtgaatgcaggcgcgagaaatggtcttggagctcctgttcgacttgtagctcctgccccagtcatcattagttcctcagctgcacaagcagctgttgcagcagccgcagcttcagcaaatggagcagctggtggtcttgctggaacaacaaatggaccatttcgccctttaggaacacagcagcctcagccccagccccagcagcagcccaataacaacctggcatccagttctttctacggcaacaactctctgaacagcaattcacagagcagctccctcttctcccagggctctgcccagcctgccaacacatccttgggattcggaagtagcagttctctcggcgccaccctgggatccgcccttggagggtttggaacagcagttgcaaactccaacactggcagtggctcccgccgtgactccctgactggcagcagtgacctttataagaggacatcgagcagcttgacccccattggacacagtttttataacggccttagcttttcctcctctcctggacccgtgggcatgcctctccctagtcagggaccaggacattcacagacaccacctccttccctctcttcacatggatcctcttcaagcttaaacctgggaggactcacgaatggcagtggaagatacatctctgctgctccaggcgctgaagccaagtaccgcagtgcaagcagcgcctccagcctcttcagcccgagcagcactcttttctcttcctctcgtttgcgatatggaatgtctgatgtcatgccttctggcaggagcaggcttttggaagattttcgaaacaaccggtaccccaatttacaactgcgggagattgctggacatataatggaattttcccaagaccagcatgggtccagattcattcagctgaaactggagcgtgccacaccagctgagcgccagcttgtcttcaatgaaatcctccaggctgcctaccaactcatggtggatgtgtttggtaattacgtcattcagaagttctttgaatttggcagtcttgaacagaagctggctttggcagaacggattcgaggccacgtcctgtcattggcactacagatgtatggctgccgtgttatccagaaagctcttgagtttattccttcagaccagcagaatgagatggttcgggaactagatggccatgtcttgaagtgtgtgaaagatcagaatggcaatcacgtggttcagaaatgcattgaatgtgtacagccccagtctttgcaatttatcatcgatgcgtttaagggacaggtatttgccttatccacacatccttatggctgccgagtgattcagagaatcctggagcactgtctccctgaccagacactccctattttagaggagcttcaccagcacacagagcagcttgtacaggatcaatatggaaattatgtaatccaacatgtactggagcacggtcgtcctgaggataaaagcaaaattgtagcagaaatccgaggcaatgtacttgtattgagtcagcacaaatttgcaagcaatgttgtggagaagtgtgttactcacgcctcacgtacggagcgcgctgtgctcatcgatgaggtgtgcaccatgaacgacggtccccacagtgccttatacaccatgatgaaggaccagtatgccaactacgtggtccagaagatgattgacgtggcggagccaggccagcggaagatcgtcatgcataagatccggccccacatcgcaactcttcgtaagtacacctatggcaagcacattctggccaagctggagaagtactacatgaagaacggtgttgacttagggcccatctgtggcccccctaatggtatcatctgaggcagtgtcacccgctgttccctcattcccgctgacctcactggcccactggcaaatccaaccagcaaccagaaatgttctagtgtagagtctgagacgggcaagtggttgctccaggattactccctcctccaaaaaaggaatcaaatccacgagtggaaaagcctttgtaaatttaattttattacacataacatgtactattttttttaattgactaattgccctgctgttttactggtgtataggatacttgtacataggtaaccaatgtacatgggaggccacatattttgttcactgttgtatctatatttcacatgtggaaactttcagggtggttggtttaacaaaaaaaaaaagctttaaaaaaaaaagaaaaaaaggaaaaggtttttagctcatttgcctggccggcaagttttgcaaatagctcttccccacctcctcattttagtaaaaaacaaacaaaaacaaaaaaacctgagaagtttgaattgtagttaaatgaccccaaactggcatttaacactgtttataaaaaatatatatatatatatatatatatataatgaaaaaggtttcagagttgctaaagcttcagtttgtgacattaagtttatgaaattctaaaaaatgccttttttggagactatattatgctgaagaaggctgttcgtgaggaggagatgcgagcacccagaacgtcttttgaggctgggcgggtgtgattgtttactgcctactggatttttttctattaacattgaaaggtaaaatctgattatttagcatgagaaaaaaaaatccaactctgcttttggtcttgcttctataaatatatagtgtatacttggtgtagactttgcatatatacaaatttgtagtattttcttgttttgatgtctaatctgtatctataatgtaccctagtagtcgaacatacttttgattgtacaattgtacatttgtatacctgtaatgtaaatgtggagaagtttgaatcaacataaacacgttttttggtaagaaaagagaattagccagccctgtgcattcagtgtatattctcaccttttatggtcgtagcatatagtgttgtatattgtaaattgtaatttcaaccagaagtaaatttttttcttttgaaggaataaatgttctttatacagcctagttaatgtttaaaaagaaaaaaatagcttggttttatttgtcatctagtctcaagtatagcgagattctttctaaatgttattcaagattgagttctcactagtgtttttttaatcctaaaaaagtaatgttttgattttgtgacagtcaaaaggacgtgcaaaagtctagccttgcccgagctttccttacaatcagagcccctctcaccttgtaaagtgtgaatcgcccttcccttttgtacagaagatgaactgtattttgcattttgtctacttgtaagtgaatgtaacatactgtcaattttccttgtttgaatatagaattgtaacactacacggtgtacatttccagagccttgtgtatatttccaatgaacttttttgcaagcacacttgtaaccatatgtgtataattaacaaacctgtgtatgcttatgcctgggcaactattttttgtaactcttgtgtagattgtctctaaacaatgtgtgatctttattttgaaaaatacagaactttggaatctgaaaaaaaaaaaaaaaaaaaaaaaaaaaaaaa

SEQ ID NO: 16 - DNA, 2594 bp, Homo sapiens
aggggccgcggagccgcggctaaggaacgcgggccgcccacccgctcccggtgcagcggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcagttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtcgagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattccttctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcaaatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaa

SEQ ID NO: 17 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 18 - DNA, 1235 bp, Homo sapiens
cccaggggccgacgggagtggcggccgcgcggaggaggccaagatggcggcagctgcggcttcgcttcgcggggtagtgttgggcccgcggggcgcggggctcccgggcgcgcgtgcccggggtctgctgtgcagcgcgcgtcccgggcagctcccgctacggacacctcaggcagtggccttgtcgtcgaagtctggcctttcccgaggccggaaagtgatgctgtcagcgctgggcatgctggcggcagggggtgcggggctggccgtggctctgcattcggctgtgagtgccagtgacctggagctgcacccccccagctatccgtggtctcaccgtggcctcctctcttccttggaccacaccagcatccggaggggtttccaggtatataagcaggtgtgcgcctcctgccacagcatggacttcgtggcctaccgccacctggtgggcgtgtgctacacggaggatgaagctaaggagctggctgcggaggtggaggttcaagacggccccaatgaagatggggagatgttcatgcggccagggaagctgttcgactatttcccaaaaccataccccaacagtgaggctgctcgagctgccaacaacggagcattgccccctgacctcagctacatcgtgcgagctaggcatggtggtgaggactacgtcttctccctgctcacgggctactgcgagccacccaccggggtgtcactgcgggaaggtctctacttcaacccctactttcctggccaggccattgccatggcccctcccatctacacagatgtcttagagtttgacgatggcaccccagctaccatgtcccagatagccaaggatgtgtgcaccttcctgcgctgggcatctgagccagagcacgaccatcgaaaacgcatggggctcaagatgttgatgatgatggctctgctggtgcccctggtctacaccataaagcggcacaagtggtcagtcctgaagagtcggaagctggcatatcggccgcccaagtgaccctgtccagtgtctgcttgccatcctgccagaacaggccctcaagcccaagagccatcccaggcctgttcaggcctcagctaagcctctcttcatctggaagaagaggcaagggggcaggagaccaggctctagctctgggccctccttcagcccccatcatgggaataaattaattttctcaatgtaaaaaaaaaaaaaaaaaaaaaaaaaa

SEQ ID NO: 19 - DNA, 1526 bp, Homo sapiens
ccggaagtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtgtaaaaaaaaaaaaaaaaa

SEQ ID NO: 20 - DNA, 2405 bp, Homo sapiens
tccggcgtggtgcgcaggcgcggtatcccccctcccccgccagctcgaccccggtgtggtgcgcaggcgcagtctgcgcagggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaaaaaaaaaaaaaaaaa

SEQ ID NO: 21 - DNA, 1852 bp, Homo sapiens
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 22 - DNA, 2321 bp, Homo sapiens
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 23 - DNA, 3734 bp, Homo sapiens
caaatgcgaacttaggctgttacacaactgctggggtctgttctcgccgcccgcccggcagtcaggcagcgtcgccgccgtggtagcagcctcagccgtttctggagtctcgggcccacagtcaccgccgcttacctgcgcctcctcgagcctccggagtccccgtccgcccgcacaggccggttcgccgtctgcgtctcccccacgccgcctcgcctgccgccgcgctcgtccctccgggccgacatgagtggggaccacctccacaacgattcccagatcgaagcggatttccgattgaatgattctcataaacacaaagataaacacaaagatcgagaacaccggcacaaagaacacaagaaggagaaggaccgggaaaagtccaagcatagcaacagtgaacataaagattctgaaaagaaacacaaagagaaggagaagaccaaacacaaagatggaagctcagaaaagcataaagacaaacataaagacagagacaaggaaaaacgaaaagaggaaaaggttcgagcctctggggatgcaaaaataaagaaggagaaggaaaatggcttctctagtccaccacaaattaaagatgaacctgaagatgatggctattttgttcctcctaaagaggatataaagccattaaagagacctcgagatgaggatgatgctgattataaacctaagaaaattaaaacagaagataccaagaaggagaagaaaagaaaactagaagaagaagaggatggtaaattgaaaaaacccaagaataaagataaagataaaaaagttcctgagccagataacaagaaaaagaagccgaagaaagaagaggaacagaagtggaaatggtgggaagaagagcgctatcctgaaggcatcaagtggaaattcctagaacataaaggtccagtatttgccccaccatatgagcctcttccagagaatgtcaagttttattatgatggtaaagtcatgaagctgagccccaaagcagaggaagtagctacgttctttgcaaaaatgctcgaccatgaatatactaccaaggaaatatttaggaaaaatttctttaaagactggagaaaggaaatgactaatgaagagaagaatattatcaccaacctaagcaaatgtgattttacccagatgagccagtatttcaaagcccagacggaagctcggaaacagatgagcaaggaagagaaactgaaaatcaaagaggagaatgaaaaattactgaaagaatatggattctgtattatggataaccacaaagagaggattgctaacttcaagatagagcctcctggacttttccgtggccgcggcaaccaccccaagatgggcatgctgaagagacgaatcatgcccgaggatataatcatcaactgtagcaaagatgccaaggttccttctcctcctccaggacataagtggaaagaagtccggcatgataacaaggttacttggctggtttcctggacagagaacatccaaggttccattaaatacatcatgcttaaccctagttcacgaatcaagggtgagaaggactggcagaaatacgagactgctcggcggctgaaaaaatgtgtggacaagatccggaaccagtatcgagaagactggaagtccaaagagatgaaagtccggcagagagctgtagccctgtacttcatcgacaagcttgctctgagagcaggcaatgaaaaggaggaaggagaaacagcggacactgtgggctgctgctcacttcgtgtggagcacatcaatctacacccagagttggatggtcaggaatatgtggtagagtttgacttcctcgggaaggactccatcagatactataacaaggtccctgttgagaaacgagtttttaagaacctacaactatttatggagaacaagcagcccgaggatgatctttttgatagactcaatactggtattctgaataagcatcttcaggatctcatggagggcttgacagccaaggtattccgtacatacaatgcctccatcacgctacagcagcagctaaaagaactgacagccccggatgagaacatcccagcgaagatcctttcttataaccgtgccaatcgagctgttgcaattctttgtaaccatcagagggcaccaccaaaaacttttgagaagtctatgatgaacttgcaaactaagattgatgccaagaaggaacagctagcagatgcccggagagacctgaaaagtgctaaggctgatgccaaggtcatgaaggatgcaaagacgaagaaggtagtagagtcaaagaagaaggctgttcagagactggaggaacagttgatgaagctggaagttcaagccacagaccgagaggaaaataaacagattgccctgggaacctccaaactcaattatctggaccctaggatcacagtggcttggtgcaagaagtggggtgtcccaattgagaagatttacaacaaaacccagcgggagaagtttgcctgggccattgacatggctgatgaagactatgagttttagccagtctcaagaggcagagttctgtgaagaggaacagtgtggtttgggaaagatggataaactgagcctcacttgccctcgtgcctgggggagagaggcagcaagtcttaacaaaccaacatctttgcgaaaagataaacctggagatattataagggagagctgagccagttgtcctatggacaacttatttaaaaatatttcagatatcaaaattctagctgtatgatttgttttgaattttgtttttattttcaagagggcaagtggatgggaatttgtcagcgttctaccaggcaaattcactgtttcactgaaatgtttggattctcttagctactgtatgcaaagtccgattatattggtgcgtttttacagttagggttttgcaataacttctatattttaatagaaataaattcctaaactcccttccctctctcccatttcaggaatttaaaattaagtagaacaaaaaacccagcgcacctgttagagtcgtcactctctattgtcatggggatcaattttcattaaacttgaagcagtcgtggctttggcagtgttttggttcagacacctgttcacagaaaaagcatgatgggaaaatatttcctgacttgagtgttcctttttaaatgtgaatttttatttctttttaattattttaaaatatttaaacctttttcttgatcttaaagatcgtgtagattggggttggggagggatgaagggcgagtgaatctaaggataatgaaataatcagtgactgaaaccattttcccatcatcctttgttctgagcattcgctgtaccctttaagatatccatctttttctttttaaccctaatctttcacttgaaagattttattgtataaaaagtttcacaggtcaataaacttagaggaaaatgagtatttggtccaaaaaaaggaaaaataatcaagattttagggcttttattttttcttttgtaattgtgtaaaaaatggaaaaaaacataaaaagcagaattttaatgtgaagacattttttgctataatcattagttttagaggcattgttagtttagtgtgtgtgcagagtccatttcccacatctttcctcaagtatcttctatttttatcatgaattcccttttaatcaactgtaggttatttaaaataaattcctacaacttaatggaaa

SEQ ID NO: 24 - DNA, 987 bp, Homo sapiens
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 25 - DNA, 1310 bp, Homo sapiens
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

(19) United States
(12) Patent Application Publication
(10) Pub. No.: US 2015/0056191 A1
(43) Pub. Date: Feb. 26, 2015

(54) IGF1 BIOMARKER FOR IGF1R INHIBITOR THERAPY

(71) Applicant: MERCK SHARP & DOHME CORP., RAHWAY, NJ (US)

(72) Inventors: Sriram Sathyanarayanan, Lexington, MA (US); Mark Ayers, Pennington, NJ (US)

(21) Appl. No.: 14/389,066

(22) PCT Filed: Mar. 25, 2013
(86) PCT No.: PCT/US13/33694

§ 371 Date: Sep. 29, 2014

Related U.S. Application Data
(60) Provisional application No. 61/617,954, filed on Mar. 30, 2012.

(51) Int. Cl. C12Q 1/68; A61K 31/439; A61K 31/7068; A61K 31/517; C07K 16/28; A61K 39/395

(52) U.S. Cl. CPC C12Q 1/6886; C07K 16/2866; A61K 39/39558; A61K 31/7068; A61K 31/517; A61K 31/439; C12Q 2600/158; C12Q 2600/106
USPC 424/133.1; 435/6.11; 506/9; 506/16

(57) ABSTRACT

The present invention provides, inter alia, methods for treating tumors that are sensitive to an IGF1R inhibitor. The tumor are determined to be sensitive if the level of IGF1 mRNA expression in the tumor cells, relative to one or more reference genes, reaches a certain threshold level. Methods for evaluating patients as candidates for receipt of the IGF1R inhibitor therapy are also provided as well along with in vitro assay methods and kits for performing any of the methods.

This application claims the benefit of U.S. provisional patent application No. 61/617,954, filed Mar. 30, 3012; which is herein incorporated by reference in its entirety.

### FIELD OF THE INVENTION

The field of the present invention provides methods for treating a tumor that expresses IGF1 at a certain threshold level with an IGF1R inhibitor.

### BACKGROUND OF THE INVENTION

The IGF1R signaling pathway has been therapeutically targeted for cancer therapy. Various antibodies and small molecules targeting IGF1R are currently undergoing various stages of clinical development. One of the key challenges in the development of targeted therapies is to identify the patient population most likely to benefit from the targeted therapies. IGF1 is a key ligand for the activation of the IGF1R pathway. Increased IGF1 levels in a tumor are a useful indicator that the tumor is sensitivity to IGF1R inhibitor therapy. The degree to which IGF1 expression levels in a tumor cell must be increased to reliably indicate that the cell is IGF1R inhibitor sensitive is not known in the art.

### SUMMARY OF THE INVENTION

The present invention provides a method for treating a tumor in a subject (e.g., a mammal such as a human; e.g., a subject having a tumor that is sensitive to IGFIR inhibitor therapy and/or likely to experience a positive clinical outcome upon treatment with an IGF1R inhibitor) in need of such treatment, that expresses IGF1 mRNA, comprising administering a therapeutically effective amount of an IGF1R inhibitor (e.g., dalotuzumab, robatumumab, figitumumab, cixutumumab, ganitumab, AVE1642, OSI-906, NVP-AEW541 or NVP-ADW742), optionally in association with a further chemotherapeutic agent, to said subject; if the fractional cycle number of a real time polymerase chain reaction amplification of IGF1 cDNA, that was reverse transcribed from IGF1 mRNA from a cell of said tumor, normalized relative to that of one or more reference genes, wherein acceleration of amplification is at a maximum (e.g., wherein the point at which acceleration of amplification is at a maximum is determined by determining the second derivative maxima of an amplification curve of the reaction), is at or below about 2.87 or at or below about 1.83 to about 2.03.

The present invention further provides a method for selecting a subject (e.g., a mammal such as a human) with a tumor (e.g., a subject having a tumor that is sensitive to IGF1R inhibitor therapy and/or likely to experience a positive clinical outcome upon treatment with an IGF1R inhibitor) for treatment with an IGF1R inhibitor (e.g., dalotuzumab, robatumumab, figitumumab, cixutumumab, ganitumab, AVE1642, OSI-906, NVP-AEW541 or NVP-ADW742), optionally in association with a further chemotherapeutic agent, comprising selecting the subject for treatment of the tumor with the IGF1R inhibitor if the fractional cycle number of a real time polymerase chain reaction amplification of IGF1 cDNA, that was reverse transcribed from IGF1 mRNA from a cell of said tumor, normalized relative to that of one or more reference genes, in which acceleration of amplification is at a maximum (e.g., wherein the point at which acceleration of amplification is at a maximum is determined by determining the second derivative maxima of an amplification curve of the reaction), is at or below about 2.87 or at or below about 1.83 to about 2.03. Optionally, the method further comprises administering a therapeutically effective amount of IGF1R inhibitor to the selected subject.

The present invention also provides a method for selecting a therapy for a subject (e.g., a mammal such as a human) with a tumor (e.g., a subject having a tumor that is sensitive to IGF1R inhibitor therapy and/or likely to experience a positive clinical outcome upon treatment with an IGF1R inhibitor) comprising selecting an IGF1R inhibitor (e.g., dalotuzumab, robatumumab, figitumumab, cixutumumab, ganitumab, AVE1642, OSI-906, NVP-AEW541 or NVP-ADW742), optionally in association with a further chemotherapeutic agent, for treatment of the tumor in the subject if the fractional cycle number of a real time polymerase chain reaction amplification of IGF1 cDNA, that was reverse transcribed from IGF1 mRNA from a cell of said tumor, normalized relative to that of one or more reference genes, in which acceleration of amplification is at a maximum (e.g., wherein the point at which acceleration of amplification is at a maximum is determined by determining the second derivative maxima of an amplification curve of the reaction), is at or below about 2.87 or at or below about 1.83 to about 2.03. Optionally, the method further comprises administering a therapeutically effective amount of IGF1R inhibitor, as the selected therapy, to the subject.

The present invention also provides a method for evaluating the sensitivity of tumor cells (e.g., in vitro tumor cells) to IGF1R inhibitor therapy comprising determining that the tumor cells are sensitive to the IGF1R inhibitor (e.g., dalotuzumab, robatumumab, figitumumab, cixutumumab, ganitumab, AVE1642, OSI-906, NVP-AEW541 or NVP-ADW742), optionally in association with a further chemotherapeutic agent, if the fractional cycle number of a real time polymerase chain reaction amplification of IGF1 cDNA, that was reverse transcribed from IGF1 mRNA from a cell of said tumor, normalized relative to that of one or more reference genes, in which acceleration of amplification is at a maximum (e.g., wherein the point at which acceleration of amplification is at a maximum is determined by determining the second derivative maxima of an amplification curve of the reaction), is at or below about 2.87 or at or below about 1.83 to about 2.03. Optionally, the method further comprises administering a therapeutically effective amount of IGF1R inhibitor to a subject (e.g., a mammal such as a human) from whom the tumor cells were obtained.

The methods of the present invention can include any one or more of the following steps, (a) obtaining cells of the subject's tumor; (b) isolating RNA from said cells; (c) generating cDNA by reverse transcribing the RNA; (d) separately amplifying the cDNA encoding IGF1 and encoding one or more reference genes selected from the group consisting of: CYC1, HMBS, TOP1, SDHA, GUSB, PUM1, HPRT1, ACTB, UBC, B2M, GAPDH, and TUBB2A while monitoring production of the cDNA during the amplification; and (e) determining the quantity amplified cDNA generated and; optionally, normalizing the determined quantity of IGF1 with that of the reference gene(s).

A further chemotherapeutic agent to be provided with an IGF1R inhibitor in a method of the present invention can be any one or more of the following: 5-fluorouridine; 131-I-TM-601; 13-cis-retinoic acid; 3-[5-(methylsulfonylpiperadinemethyl)-indolyl]-quinolone; 40-O-(2-hydroxyethyl)-rapamycin; 4-hydroxytamoxifen; 5-deooxyuridine; 6-mercaptopurine; 7-hydroxy staurosporine; a combination of irinotecan, 5-fluorouracil and leucovorin; a combination of oxaliplatin fluorouracil and folinic acid; A-443654; abiraterone acetate; abraxane; ABT-578; acolbifene; ADS-100380; AG-013736; alprazolam; ALT-110; altretamine; amifostine; aminoglutethimide; AMN-107; amrubicin; amsacrine; an antiandrogen; anagrelide; anastrazole; angiostatin; an EGF Receptor antagonuist; an selective estrogen receptor modulator (SERM); an AKT inhibitor; an anti-angiogenesis agents; an anti-EGFR antibody; an anti-emetic; an anti-HER2 antibody; an anti-VEGF antibody; an aromatase inhibitor; an CDK inhibitor; an CYP17 lyase inhibitor; an estrogen; an GnRH agonists; a HER2 antagonist; a lutenizing hormone-releasing hormone agonist; an MEK inhibitor; an mTOR inhibitor; an NK-1 receptor antagonists; a PI3 kinase inhibitor; a progestational agent; a Raf inhibitor; a VEGFR inhibitor; AP-23573; aprepitant; ARQ-197; arzoxifene; AS-252424; AS-605240; asparaginase; AT-9263; atrasentan; AV-299; AZD 1152; AZD 6244; azd2171; AZD-6244; Bacillus Calmette-Guerin vaccine; batabulin; BC-210; bevacizumab; bicalutamide; Bio 111; BIO 140; bleomycin; BMS-214662; BMS-247550; BMS-275291; BMS-310705; bortezimib; buserelin; busulfan; calcitriol; camptothecin; canertinib; capecitabine; carboplatin; carmustine; casopitant; CC 8490; CG-1521; CG-781; chlamydocin; chlorambucil; cilengitide; cimitidine; cisplatin; Cladribine; clodronate; COL-3; conjugated estrogens; CP-724714; cyclophosphamide; cyproterone; cyproterone acetate; cytarabine; cytosine arabinoside; cytproterone acetate; dacarbazine; dactinomycin; darbepoetin alfa; dasatanib; daunorubicin; decatanib; deguelin; denileukin; deoxycoformycin; depsipeptide; DES(diethylstilbestrol); dexamethasone; diarylpropionitrile; diethylstilbestrol; diftitox; diphenhydramine; DN-101; docetaxel; dolasetron; dovitinib; doxorubicin; doxorubicin HCl liposome injection; droloxifene; dronabinol; droperidol; edotecarin; edotreotide (yttrium-90 labeled or unlabeled); EKB-569; EMD121974; endostatin; enzastaurin; epirubicin; epithilone B; epoetin alfa; ERA-923; erbitux; erlotinib; erythropoietin; estradiol; estramustine; etoposide; everolimus; exemestane; finasteride; flavopiridol; floxuridine; fludarabine; fludrocortisones; fluoxymesterone; flutamide; fulvestrant; galeterone; GDC-0941; gefitinib; gemcitabine; a combination of gemcitabine in association with erlotinib; gimatecan; goserelin; goserelin acetate; gossypol; granisetron; GSK461364; GSK690693; GW-572016; haloperidol; HKI-272; HMR-3339; hydroxyprogesterone caproate; hydroxyurea; hydroxyzine; IC87114; idarubicin; Idoxifene; ifosfamide; IL13-PE38QQR; IM862; imatinib; IMC-1C11; INO 1001; interferon; interleukin-12; IPdR; ipilimumab; irinotecan; JNJ-16241199; ketoconazole; KRN951; KRX-0402; L-779,450; lapatanib; lasofoxifene; Lep-etu; letrozole; leucovorin; leuprolide; leuprolide acetate; levamisole; lomustine; lonafarnib; lorazepam; Lucanthone; LY 317615; LY292223; LY292696; LY293646; LY293684; LY294002; marimastat; MDV-3100; mechlorethamine; medroxyprogesterone acetate; megestrol acetate; melphalan; mercaptopurine; mesna; methotrexate; methylprednisolone; metoclopramide; mithramycin; mitomycin; mitotane; mitoxantrone; MK-0457; MLN8054; neovastat; netupitant; neuradiab; nilotinib; nilutimide; nolatrexed; NVP-BEZ235; NVP-LAQ824; oblimersen; octreotide; ofatumumab; ON 0910.Na; ondansetron; oregovomab; orteronel; oxaliplatin; paclitaxel; palonosetron; pamidronate; panitumumab; pazopanib; PD0325901; PEG-filgrastim; PEG-interferon; PEG-labeled irinotecan; pemetrexed; pentostatin; perifosine; PHA-739358; phenylalanine mustard; PI-103; PIK-75; pipendoxifene; PKI-166; plicamycin; porfimer; prednisone; procarbazine; prochlorperazine; progestins; PTK787/ZK 222584; PX-866; R-763; RAD001; raloxifene; raltitrexed; razoxin; ridaforolimus; rituximab; romidepsin; RTA 744; rubitecan; scriptaid; Sdx 102; seliciclib; semaxanib; SF1126; sirolimus; SN36093; sorafenib; spironolactone; squalamine; SR13668; streptozocin; SU6668; suberoyl analide hydroxamic acid; sunitinib; sunitinib malate; talampanel; tamoxifen; temozolomide; temsirolimus; teniposide; tesmilifene; testosterone; tetrandrine; TGX-221; thalidomide; thioguanine; thiotepa; ticilimumab; tipifarnib; TKI-258; TLK 286; topotecan; toremifene citrate; trabectedin; trastuzumab; tretinoin; trichostatin A; triciribine phosphate monohydrate; triptorelin pamoate; tropisetron; TSE-424; uracil mustard; valproic acid; valrubicin; vandetanib; vatalanib; VEGF trap; vinblastine; vincristine; vindesine; vinorelbine; vitaxin; vitespan; vorinostat; VX-745; wortmannin; Xr 311; zanolimumab; ZK186619; ZK-304709, ZM336372; ZSTK474;

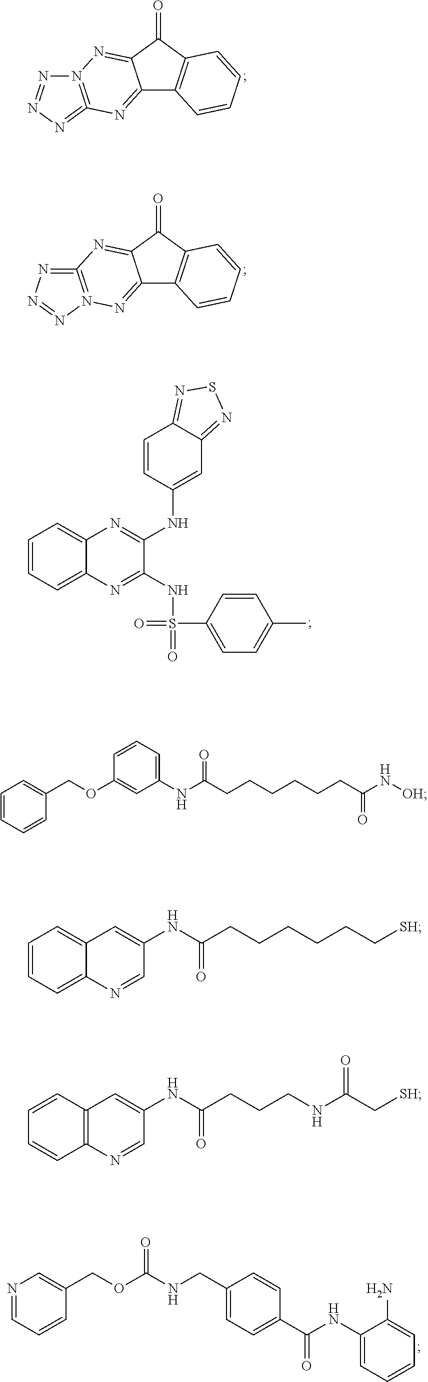

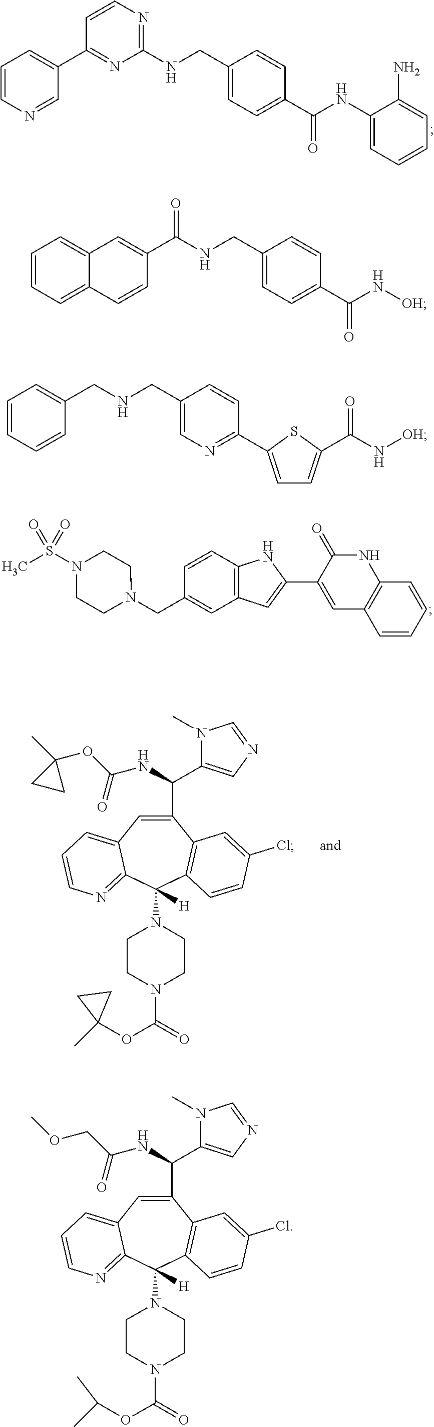

The various methods of using the IGF1 biomarker that are disussed herein (e.g., methods of treatment or methods of evaluating a subject for treatment with an IGF1R inhibitor), optionally further comprising evaluating the expression level of KRAS and/or evaluating whether the KRAS is wild-type or mutated, in connection with any of the specific IGF1R inhibitors that are discussed herein (e.g., under the IGF1R Inhibitors section herein, e.g., dalotuzumab), optionally in association with any 1, 2 or 3 of the specific further chemotherapeutic agents discussed herein (e.g., under the Further Chemotherapeutics section herein; e.g., ridaforolimus), wherein the method of using the biomarker is in connection with a subject suffering from any of osteosarcoma, rhabdomyosarcoma, neuroblastoma, kidney cancer, leukemia, renal transitional cell cancer, bladder cancer, Wilm's cancer, ovarian cancer, pancreatic cancer (e.g., where in the subject is administered the IGF1R inhibitor (e.g., MK0646) in association with gemcitabine, and optionally, ridaforolimus), breast cancer, prostate cancer, bone cancer, lung cancer, gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer; are encompassed by the present invention.

### BRIEF DESCRIPTION OF THE FIGURES

FIG. 1: H2122 NSCLC cells were grown in the presence and absence of IGF1 and MK-0646 and relative cell growth was compared. A statistically significant growth inhibition by MK-0646 treatment was observed in H2122 cell grown in the presence of the ligand IGF1.

FIG. 2. High IGF1 identifies MK0646 responsive tumors in colorectal cancer primary tumor xenograft models.

FIG. 3. In silica BLAST analysis with the IGF1 forward & reverse primer sequences

FIG. 4. Electropherogram showing specific product for IGF1.

FIG. 5(A-B). Reverse transcription polymerase chain reaction (RT-PCR) amplification curve and standard curve.

FIG. 6. IGF1 expression levels in tumor tissue of colorectal cancer patients.

FIG. 7. Kaplan-Meier Curve: progression free survival (PFS) of patients with high and low IGF1 expression levels in tumor tissue.

FIG. 8. Kaplan-Meier Curve: overall survival (OS) of patients with high and low IGF1 expression levels in tumor tissue.

### DETAILED DESCRIPTION OF THE INVENTION

The present invention comprises methods for treating a subset of patients suffering from a tumor that expresses IGF1 using an IGF1R inhibitor (e.g., dalotuzumab). An evaluation of the IGF1 mRNA expression levels in a population of tumors has identified a clear distinction between tumors that are highly sensitive to IGF1R inhibitors and those that are less responsive. The cut point between the highly sensitive and less sensitive subpopulations of tumors can be expressed in terms of IGF1 mRNA expression levels relative to any of a number of reference genes as determined by real time PCR amplification of IGF1 cDNA that was generated by reverse transcription of IGF1 mRNA from cells of the tumors.

In a certain embodiment of the invention, measurement of IGF1 RNA and/or protein in the serum of a patient with a tumor, in the practice of a methods described herein, is specifically excluded from the invention.

A “subject” or “patient” or the like is a mammal such as a human, monkey, primate, canine, feline, rat, rabbit or mouse.

When the cells of a tumor are analyzed to determine IGF1 expression, in an embodiment of the invention, tumor cells or both stromal cells and tumor cells are analyzed.

“IGF1” is insulin-like growth factor 1, for example, human insulin-like growth factor 1. In an embodiment of the invention, human IGF1 comprises the nucleotide sequence or nucleotides 220-696 thereof (see Genbank accession no. NM_{—}001111283):

IGF1 >nucfasta||NM_001111283 NM_001111283 1
(SEQ ID NO: 13)
ttttgtagataaatgtgaggattttctctaaatccctcttctgtt
tgctaaatctcactgtcactgctaaattcagagcagatagagcct
gcgcaatggaataaagtcctcaaaattgaaatgtgacattgctct
caacatctcccatctctctggatttctttttgcttcattattcct
gctaaccaattcattttcagactttgtacttcagaagcaatggga
aaaatcagcagtcttccaacccaattatttaagtgctgcttttgt
gatttcttgaaggtgaagatgcacaccatgtcctcctcgcatctc
ttctacctggcgctgtgcctgctcaccttcaccagctctgccacg
gctggaccggagacgctctgcggggctgagctggtggatgctctt
cagttcgtgtgtggagacaggggcttttatttcaacaagcccaca
gggtatggctccagcagtcggagggcgcctcagacaggcatcgtg
gatgagtgctgcttccggagctgtgatctaaggaggctggagatg
tattgcgcacccctcaagcctgccaagtcagctcgctctgtccgt
gcccagcgccacaccgacatgcccaagacccagaagtatcagccc
ccatctaccaacaagaacacgaagtctcagagaaggaaaggaagt
acatttgaagaacgcaagtagagggagtgcaggaaacaagaacta
caggatgtaggaagaccctcctgaggagtgaagagtgacatgcca
ccgcaggatcctttgctctgcacgagttacctgttaaactttgga
acacctaccaaaaaataagtttgataacatttaaaagatgggcgt
ttcccccaatgaaatacacaagtaaacattccaacattgtcttta
ggagtgatttgcaccttgcaaaaatggtcctggagttggtagatt
gctgttgatcttttatcaataatgttctatagaaaagaaaaaaaa
aatatatatatatatatatcttagtccctgcctctcaagagccac
aaatgcatgggtgttgtatagatccagttgcactaaattcctctc
tgaatcttggctgctggagccattcattcagcaaccttgtctaag
tggtttatgaattgtttccttatttgcacttctttctacacaact
cgggctgtttgttttacagtgtctgataatcttgttagtctatac
ccaccacctcccttcataacctttatatttgccgaatttggcctc
ctcaaaagcagcagcaagtcgtcaagaagcacaccaattctaacc
cacaagattccatctgtggcatttgtaccaaatataagttggatg
cattttattttagacacaaagctttatttttccacatcatgctta
caaaaaagaataatgcaaatagttgcaactttgaggccaatcatt
tttaggcatatgttttaaacatagaaagtttcttcaactcaaaag
agttccttcaaatgatgagttaatgtgcaacctaattagtaactt
tcctctttttattttttccatatagagcactatgtaaatttagca
tatcaattatacaggatatatcaaacagtatgtaaaactctgttt
tttagtataatggtgctattttgtagtttgttatatgaaagagtc
tggccaaaacggtaatacgtgaaagcaaaacaataggggaagcct
ggagccaaagatgacacaaggggaagggtactgaaaacaccatcc
atttgggaaagaaggcaaagtccccccagttatgccttccaagag
gaacttcagacacaaaagtccactgatgcaaattggactggcgag
tccagagaggaaactgtggaatggaaaaagcagaaggctaggaat
tttagcagtcctggtttctttttctcatggaagaaatgaacatct
gccagctgtgtcatggactcaccactgtgtgaccttgggcaagtc
acttcacctctctgtgcctcagtttcctcatctgcaaaatggggg
caatatgtcatctacctacctcaaaggggtggtataaggtttaaa
aagataaagattcagattttttttaccctgggttgctgtaagggt
gcaacatcagggcgcttgagttgctgagatgcaaggaattctata
aataacccattcatagcatagctagagattggtgaattgaatgct
cctgacatctcagttcttgtcagtgaagctatccaaataactggc
caactagttgttaaaagctaacagctcaatctcttaaaacacttt
tcaaaatatgtgggaagcatttgattttcaatttgattttgaatt
ctgcatttggttttatgaatacaaagataagtgaaaagagagaaa
ggaaaagaaaaaggagaaaaacaaagagatttctaccagtgaaag
gggaattaattactctttgttagcactcactgactcttctatgca
gttactacatatctagtaaaacctcgtttaatactataaataata
ttctattcattttgaaaaacacaatgattccttcttttctaggca
atataaggaaagtgatccaaaatttgaaatattaaaataatatct
aataaaaagtcacaaagttatcttctttaacaaactttactctta
ttcttagctgtatatacatttttttaaaagtttgttaaaatatgc
ttgactagagtttccagttgaaaggcaaaaacttccatcacaaca
agaaatttcccatgcctgctcagaagggtagcccctagctctctg
tgaatgtgttttatccattcaactgaaaattggtatcaagaaagt
ccactggttagtgtactagtccatcatagcctagaaaatgatccc
tatctgcagatcaagattttctcattagaacaatgaattatccag
cattcagatctttctagtcaccttagaactttttggttaaaagta
cccaggcttgattatttcatgcaaattctatattttacattcttg
gaaagtctatatgaaaaacaaaaataacatcttcagtttttctcc
cactgggtcacctcaaggatcagaggccaggaaaaaaaaaaaaaa
gactccctggatctctgaatatatgcaaaaagaaggccccattta
gtggagccagcaatcctgttcagtcaacaagtattttaactctca
gtccaacattatttgaattgagcacctcaagcatgcttagcaatg
ttctaatcactatggacagatgtaaaagaaactatacatcatttt
tgccctctgcctgttttccagacatacaggttctgtggaataaga
tactggactcctcttcccaagatggcacttctttttatttcttgt
ccccagtgtgtaccttttaaaattattccctctcaacaaaacttt
ataggcagtcttctgcagacttaacgtgttttctgtcatagttag
atgtgataattctaagagtgtctatgacttatttccttcacttaa
ttctatccacagtcaaaaatcccccaaggaggaaagctgaaagat
gcactgccatattatctttcttaactttttccaacacataatcct
ctccaactggattataaataaattgaaaataactcattataccaa
ttcactattttattttttaatgaattaaaactagaaaacaaattg
atgcaaaccctggaagtcagttgattactatatactacagcagaa
tgactcagatttcatagaaaggagcaaccaaaatgtcacaaccca
aaactttacaagctttgcttcagaattagattgctttataattct
tgaatgaggcaatttcaagatatttgtaaaagaacagtaaacatt
ggtaagaatgagctttcaactcataggcttatttccaatttaatt
gaccatactggatacttaggtcaaatttctgttctctcttcccca
aataatattaaagtattatttgaactttttaagatgaggcagttc
ccctgaaaaagttaatgcagctctccatcagaatccactcttcta
gggatatgaaaatctcttaacacccaccctacatacacagacaca
cacacacacacacacacacacacacacacacacattcaccctaag
gatccaatggaatactgaaaagaaatcacttccttgaaaatttta
ttaaaaaacaaacaaacaaacaaaaagcctgtccacccttgagaa
tccttcctctccttggaacgtcaatgtttgtgtagatgaaaccat
ctcatgctctgtggctccagggtttctgttactattttatgcact
tgggagaaggcttagaataaaagatgtagcacattttgctttccc
atttattgtttggccagctatgccaatgtggtgctattgtttctt
taagaaagtacttgactaaaaaaaaaagaaaaaaagaaaaaaaag
aaagcatagacatatttttttaaagtataaaaacaacaattctat
agatagatggcttaataaaatagcattaggtctatctagccacca
ccacctttcaactttttatcactcacaagtagtgtactgttcacc
aaattgtgaatttgggggtgcaggggcaggagttggaaatttttt
aaagttagaaggctccattgttttgttggctctcaaacttagcaa
aattagcaatatattatccaatcttctgaacttgatcaagagcat
ggagaataaacgcgggaaaaaagatcttataggcaaatagaagaa
tttaaaagataagtaagttccttattgatttttgtgcactctgct
ctaaaacagatattcagcaagtggagaaaataagaacaaagagaa
aaaatacatagatttacctgcaaaaaatagcttctgccaaatccc
ccttgggtattctttggcatttactggtttatagaagacattctc
ccttcacccagacatctcaaagagcagtagctctcatgaaaagca
atcactgatctcatttgggaaatgttggaaagtatttccttatga
gatgggggttatctactgataaagaaagaatttatgagaaattgt
tgaaagagatggctaacaatctgtgaagattttttgtttcttgtt
tttgttttttttttttttttactttatacagtctttatgaatttc
ttaatgttcaaaatgacttggttcttttcttctttttttatatca
gaatgaggaataataagttaaacccacatagactctttaaaacta
taggctagatagaaatgtatgtttgacttgttgaagctataatca
gactatttaaaatgttttgctatttttaatcttaaaagattgtgc
taatttattagagcagaacctgtttggctctcctcagaagaaaga
atctttccattcaaatcacatggctttccaccaatattttcaaaa
gataaatctgatttatgcaatggcatcatttattttaaaacagaa
gaattgtgaaagtttatgcccctcccttgcaaagaccataaagtc
cagatctggtaggggggcaacaacaaaaggaaaatgttgttgatt
cttggttttggattttgttttgttttcaatgctagtgtttaatcc
tgtagtacatatttgcttattgctattttaatattttataagacc
ttcctgttaggtattagaaagtgatacatagatatcttttttgtg
taatttctatttaaaaaagagagaagactgtcagaagctttaagt
gcatatggtacaggataaagatatcaatttaaataaccaattcct
atctggaacaatgcttttgttttttaaagaaacctctcacagata
agacagaggcccaggggatttttgaagctgtctttattctgcccc
catcccaacccagcccttattattttagtatctgcctcagaattt
tatagagggctgaccaagctgaaactctagaattaaaggaacctc
actgaaaacatatatttcacgtgttccctctttttttttttcctt
tttgtgagatggggtctcgcactgtcccccaggctggagtgcagt
ggcatgatctcggctcactgcaacctccacctcctgggtttaagc
gattctcctgcctcagcctcctgagtagctgggattacaggcacc
caccactatgcccggctaattttttggatttttaatagagacggg
gttttaccatgttggccaggttggtctcaaactcctgaccttgtg
atttgcccgcctcagcctcccaaattgctgggattacaggcatga
gccaccacaccctgcccatgtgttccctcttaatgtatgattaca
tggatcttaaacatgatccttctctcctcattcttcaactatctt
tgatggggtctttcaaggggaaaaaaatccaagcttttttaaagt
aaaaaaaaaaaaagagaggacacaaaaccaaatgttactgctcaa
ctgaaatatgagttaagatggagacagagtttctcctaataaccg
gagctgaattacctttcactttcaaaaacatgaccttccacaatc
cttagaatctgcctttttttatattactgaggcctaaaagtaaac
attactcattttattttgcccaaaatgcactgatgtaaagtagga
aaaataaaaacagagctctaaaatccctttcaagccacccattga
ccccactcaccaactcatagcaaagtcacttctgttaatccctta
atctgattttgtttggatatttatcttgtacccgctgctaaacac
actgcaggagggactctgaaacctcaagctgtctacttacatctt
ttatctgtgtctgtgtatcatgaaaatgtctattcaaaatatcaa
aacctttcaaatatcacgcagcttatattcagtttacataaaggc
cccaaataccatgtcagatctttttggtaaaagagttaatgaact
atgagaattgggattacatcatgtattttgcctcatgtattttta
tcacacttataggccaagtgtgataaataaacttacagacactga
attaatttcccctgctactttgaaaccagaaaataatgactggcc
attcgttacatctgtcttagttgaaaagcatattttttattaaat
taattctgattgtatttgaaattattattcaattcacttatggca
gaggaatatcaatcctaatgacttctaaaaatgtaactaattgaa
tcattatcttacatttactgtttaataagcatattttgaaaatgt
atggctagagtgtcataataaaatggtatatctttctttagtaat
tacattaaaattagtcatgtttgattaattagttc

A “reference gene” is a gene whose expression is known not to increase or decrease significantly in a tumor cell of a given type (e.g., breast, lung, colorectal or any of the tumor types discussed herein) as compared to the corresponding normal, non-tumor cell. Examples of such reference genes include CYC1, HMBS, TOP1, SDHA, GUSH, PUM1, HPRT1, ACTS, UBC, B2M, GAPDH, and TUBB2A.

In an embodiment of the invention TUBB2A comprises the nucleotide sequence or nucleotides 87-1424 thereof (see Genbank accession no. NM_{—}001069):

TUBB2A >nucfasta||NM_001069 NM_001069 2
(SEQ ID NO: 14)
gcggcaggtctctgcgcagcccagcccgccggtccacgccgcgcaccg
ctccgagggccagcgccacccgctccgcagccggcaccatgcgcgaga
tcgtgcacatccaggcgggccagtgcggcaaccagatcggcgccaagt
tttgggaggtcatcagcgatgagcatgggatcgaccccacaggcagtt
accatggagacagtgacttgcagctggagagaatcaacgtgtactaca
atgaggctgctggtaacaaatatgtacctcgggccatcctggtggatc
tggagcctggcaccatggactctgtcaggtctggacccttcggccaga
tcttcagaccagacaacttcgtgttcggccagagtggagccgggaata
actgggccaagggccactacacagagggagccgagctggtcgactcgg
tcctggatgtggtgaggaaggagtcagagagctgtgactgtctccagg
gcttccagctgacccactctctggggggcggcacggggtccgggatgg
gcaccctgctcatcagcaagatccgggaagagtacccagaccgcatca
tgaacaccttcagcgtcatgccctcacccaaggtgtcagacacggtgg
tggagccctacaacgccaccctctctgtccaccagctggtggaaaaca
cagatgaaacctactccattgataacgaggccctgtatgacatctgct
tccgcaccctgaagctgaccacccccacctacggggacctcaaccacc
tggtgtcggccaccatgagcggggtcaccacctgcctgcgcttcccgg
gccagctgaacgcagacctgcgcaagctggcggtgaacatggtgccct
tccctcgcctgcacttcttcatgcccggcttcgcgcccctgaccagcc
ggggcagccagcagtaccgggcgctcacggtgcccgagctcacccagc
agatgttcgactccaagaacatgatggccgcctgcgacccgcgccacg
gccgctacctgacggtggctgccatcttccggggccgcatgtccatga
aggaggtggacgagcagatgctcaacgtgcagaacaagaacagcagct
acttcgtggagtggatccccaacaacgtgaagacggccgtgtgcgaca
tcccgccccgcggcctgaagatgtcggccaccttcatcggcaacagca
cggccatccaggagctgttcaagcgcatctccgagcagttcacggcca
tgttccggcgcaaggccttcctgcactggtacacgggcgagggcatgg
acgagatggagttcaccgaggccgagagcaacatgaacgacctggtgt
ccgagtaccagcagtaccaggacgccacggccgacgaacaaggggagt
tcgaggaggaggagggcgaggacgaggcttaaaaacttctcagatcaa
tcgtgcatccttagtgaacttctgttgtcctcaagcatggtctttcta
cttgtaaactatggtgctcagttttgcctctgttagaaattcacactg
ttgatgtaatgatgtggaactcctctaaaaattacagtattgtctgtg
aaggtatctatactaataaaaaagcatgtgtagaaaa

In an embodiment of the invention PUM1 comprises the nucleotide sequence or nucleotides 114-3674 thereof (see Genbank accession no. NM_{—}014676):

PUM1 >nucfasta||NM_014676 NM_014676 2
(SEQ ID NO: 15)
agtgggccgccatgttgtcggagtgaaaggtaagggggagcgagagcg
ccagagagagaagatcggggggctgaaatccatcttcatcctaccgct
ccgcccgtgttggtggaatgagcgttgcatgtgtcttgaagagaaaag
cagtgctttggcaggactctttcagcccccacctgaaacatcaccctc
aagaaccagctaatcccaacatgcctgttgttttgacatctggaacag
ggtcgcaagcgcagccacaaccagctgcaaatcaggctcttgcagctg
ggactcactccagccctgtcccaggatctataggagttgcaggccgtt
cccaggacgacgctatggtggactacttctttcagaggcagcatggtg
agcagcttgggggaggaggaagtggaggaggcggctataataatagca
aacatcgatggcctactggggataacattcatgcagaacatcaggtgc
gttccatggatgaactgaatcatgattttcaagcacttgctctggagg
gaagagcgatgggagagcagctcttgccaggtaaaaagttttgggaaa
cagatgaatccagcaaagatggaccaaaaggaatattcctgggtgatc
aatggcgagacagtgcctggggaacatcagatcattcagtttcccagc
caatcatggtgcagagaagacctggtcagagtttccatgtgaacagtg
aggtcaattctgtactgtccccacgatcggagagtgggggactaggcg
ttagcatggtggagtatgtgttgagctcatccccgggcgattcctgtc
taagaaaaggaggatttggcccaagggatgcagacagtgatgaaaacg
acaaaggtgaaaagaagaacaagggtacgtttgatggagataagctag
gagatttgaaggaggagggtgatgtgatggacaagaccaatggtttac
cagtgcagaatgggattgatgcagacgtcaaagattttagccgtaccc
ctggtaattgccagaactctgctaatgaagtggatcttctgggtccaa
accagaatggttctgagggcttagcccagotgaccagcaccaatggtg
ccaagcctgtggaggatttctccaacatggagtcccagagtgtcccct
tggaccccatggaacatgtgggcatggagcctcttcagtttgattatt
caggcacgcaggtacctgtggactcagcagcagcaactgtgggacttt
ttgactacaattctcaacaacagctgttccaaagacctaatgcgcttg
ctgtccagcagttgacagctgctcagcagcagcagtatgcactggcag
ctgctcatcagccgcacatcggtttagctcccgctgcgtttgtcccca
atccatacatcatcagcgctgctcccccagggacggacccctacacag
ctggattggctgcagcagcgacactaggcccagctgtggtccctcacc
agtattatggagttactccctggggagtctaccctgccagtcttttcc
agcagcaagctgccgctgccgctgcagcaactaattcagctaatcaac
agaccaccccacaggctcagcaaggacagcagcaggttctccgtggag
gagccagccaacgtcctttgaccccaaaccagaaccagcagggacagc
aaacggatccccttgtggcagctgcagcagtgaattctgcccttgcat
ttggacaaggtctggcagcaggcatgccaggttatccggtgttggctc
ctgctgcttactatgaccaaactggtgcccttgtagtgaatgcaggcg
cgagaaatggtcttggagctcctgttcgacttgtagctcctgccccag
tcatcattagttcctcagctgcacaagcagctgttgcagcagccgcag
cttcagcaaatggagcagctggtggtcttgctggaacaacaaatggac
catttcgccctttaggaacacagcagcctcagccccagccccagcagc
agcccaataacaacctggcatccagttctttctacggcaacaactctc
tgaacagcaattcacagagcagctccctcttctcccagggctctgccc
agcctgccaacacatccttgggattcggaagtagcagttctctcggcg
ccaccctgggatccgcccttggagggtttggaacagcagttgcaaact
ccaacactggcagtggctcccgccgtgactccctgactggcagcagtg
acctttataagaggacatcgagcagcttgacccccattggacacagtt
tttataacggccttagcttttcctcctctcctggacccgtgggcatgc
ctctccctagtcagggaccaggacattcacagacaccacctccttccc
tctcttcacatggatcctcttcaagcttaaacctgggaggactcacga
atggcagtggaagatacatctctgctgctccaggcgctgaagccaagt
accgcagtgcaagcagcgcctccagcctcttcagcccgagcagcactc
ttttctcttcctctcgtttgcgatatggaatgtctgatgtcatgcctt
ctggcaggagcaggcttttggaagattttcgaaacaaccggtacccca
atttacaactgcgggagattgctggacatataatggaattttcccaag
accagcatgggtccagattcattcagctgaaactggagcgtgccacac
cagctgagcgccagcttgtcttcaatgaaatcctccaggctgcctacc
aactcatggtggatgtgtttggtaattacgtcattcagaagttctttg
aatttggcagtcttgaacagaagctggctttggcagaacggattcgag
gccacgtcctgtcattggcactacagatgtatggctgccgtgttatcc
agaaagctcttgagtttattccttcagaccagcagaatgagatggttc
gggaactagatggccatgtcttgaagtgtgtgaaagatcagaatggca
atcacgtggttcagaaatgcattgaatgtgtacagccccagtctttgc
aatttatcatcgatgcgtttaagggacaggtatttgccttatccacac
atccttatggctgccgagtgattcagagaatcctggagcactgtctcc
ctgaccagacactccctattttagaggagcttcaccagcacacagagc
agcttgtacaggatcaatatggaaattatgtaatccaacatgtactgg
agcacggtcgtcctgaggataaaagcaaaattgtagcagaaatccgag
gcaatgtacttgtattgagtcagcacaaatttgcaagcaatgttgtgg
agaagtgtgttactcacgcctcacgtacggagcgcgctgtgctcatcg
atgaggtgtgcaccatgaacgacggtccccacagtgccttatacacca
tgatgaaggaccagtatgccaactacgtggtccagaagatgattgacg
tggcggagccaggccagcggaagatcgtcatgcataagatccggcccc
acatcgcaactcttcgtaagtacacctatggcaagcacattctggcca
agctggagaagtactacatgaagaacggtgttgacttagggcccatct
gtggcccccctaatggtatcatctgaggcagtgtcacccgctgttccc
tcattcccgctgacctcactggcccactggcaaatccaaccagcaacc
agaaatgttctagtgtagagtctgagacgggcaagtggttgctccagg
attactccctcctccaaaaaaggaatcaaatccacgagtggaaaagcc
tttgtaaatttaattttattacacataacatgtactattttttttaat
tgactaattgccctgctgttttactggtgtataggatacttgtacata
ggtaaccaatgtacatgggaggccacatattttgttcactgttgtatc
tatatttcacatgtggaaactttcagggtggttggtttaacaaaaaaa
aaaagctttaaaaaaaaaagaaaaaaaggaaaaggtttttagctcatt
tgcctggccggcaagttttgcaaatagctcttccccacctcctcattt
tagtaaaaaacaaacaaaaacaaaaaaacctgagaagtttgaattgta
gttaaatgaccccaaactggcatttaacactgtttataaaaaatatat
atatatatatatatatatataatgaaaaaggtttcagagttgctaaag
cttcagtttgtgacattaagtttatgaaattctaaaaaatgccttttt
tggagactatattatgctgaagaaggctgttcgtgaggaggagatgcg
agcacccagaacgtcttttgaggctgggcgggtgtgattgtttactgc
ctactggatttttttctattaacattgaaaggtaaaatctgattattt
agcatgagaaaaaaaaatccaactctgcttttggtcttgcttctataa
atatatagtgtatacttggtgtagactttgcatatatacaaatttgta
gtattttcttgttttgatgtctaatctgtatctataatgtaccctagt
agtcgaacatacttttgattgtacaattgtacatttgtatacctgtaa
tgtaaatgtggagaagtttgaatcaacataaacacgttttttggtaag
aaaagagaattagccagccctgtgcattcagtgtatattctcaccttt
tatggtcgtagcatatagtgttgtatattgtaaattgtaatttcaacc
agaagtaaatttttttcttttgaaggaataaatgttctttatacagcc
tagttaatgtttaaaaagaaaaaaatagcttggttttatttgtcatct
agtctcaagtatagcgagattctttctaaatgttattcaagattgagt
tctcactagtgtttttttaatcctaaaaaagtaatgttttgattttgt
gacagtcaaaaggacgtgcaaaagtctagccttgcccgagctttcctt
acaatcagagcccctctcaccttgtaaagtgtgaatcgcccttccctt
ttgtacagaagatgaactgtattttgcattttgtctacttgtaagtga
atgtaacatactgtcaattttccttgtttgaatatagaattgtaacac
tacacggtgtacatttccagagccttgtgtatatttccaatgaacttt
tttgcaagcacacttgtaaccatatgtgtataattaacaaacctgtgt
atgcttatgcctgggcaactattttttgtaactcttgtgtagattgtc
tctaaacaatgtgtgatctttattttgaaaaatacagaactttggaat
ctgaaaaaaaaaaaaaaaaaaaaaaaaaaaaaaa

In an embodiment of the invention UBC comprises the nucleotide sequence or nucleotides 459-2516 thereof (see Genbank accession no. NM_{—}021009):

UBC >nucfasta||NM_021009 NM_021009 5
(SEQ ID NO: 16)
aggggccgcggagccgcggctaaggaacgcgggccgcccacccgctcc
cggtgcascggcctccgcgccgggttttggcgcctcccgcgggcgccc
ccctcctcacggcgagcgctgccacgtcagacgaagggcgcagcgagc
gtcctgatccttccgcccggacgctcaggacagcggcccgctgctcat
aagactcggccttagaaccccagtatcagcagaaggacattttaggac
gggacttgggtgactctagggcactggttttctttccagagagcggaa
caggcgaggaaaagtagtcccttctcggcgattctgcggagggatctc
cgtggggcggtgaacgccgatgattatataaggacgcgccgggtgtgg
cacagctagttccgtcgcagccgggatttgggtcgcagttcttgtttg
tggatcgctgtgatcgtcacttgacaatgcagatcttcgtgaagactc
tgactggtaagaccatcaccctcgaggttgagcccagtgacaccatcg
agaatgtcaaggcaaagatccaagataaggaaggcatccctcctgacc
agcagaggctgatctttgctggaaaacagctggaagatgggcgcaccc
tgtctgactacaacatccagaaagagtccaccctgcacctggtgctcc
gtctcagaggtgggatgcaaatcttcgtgaagacactcactggcaaga
ccatcacccttgaggtcgagcccagtgacaccatcgagaacgtcaaag
caaagatccaggacaaggaaggcattcctcctgaccagcagaggttga
tctttgccggaaagcagctggaagatgggcgcaccctgtctgactaca
acatccagaaagagtctaccctgcacctggtgctccgtctcagaggtg
ggatgcagatcttcgtgaagaccctgactggtaagaccatcaccctcg
aggtggagcccagtgacaccatcgagaatgtcaaggcaaagatccaag
ataaggaaggcattccttctgatcagcagaggttgatctttgccggaa
aacagctggaagatggtcgtaccctgtctgactacaacatccagaaag
agtccaccttgcacctggtactccgtctcagaggtgggatgcaaatct
tcgtgaagacactcactggcaagaccatcacccttgaggtcgagccca
gtgacactatcgagaacgtcaaagcaaagatccaagacaaggaaggca
ttcctcctgaccagcagaggttgatctttgccggaaagcagctggaag
atgggcgcaccctgtctgactacaacatccagaaagagtctaccctgc
acctggtgctccgtctcagaggtgggatgcagatcttcgtgaagaccc
tgactggtaagaccatcactctcgaagtggagccgagtgacaccattg
agaatgtcaaggcaaagatccaagacaaggaaggcatccctcctgacc
agcagaggttgatctttgccggaaaacagctggaagatggtcgtaccc
tgtctgactacaacatccagaaagagtccaccttgcacctggtgctcc
gtctcagaggtgggatgcagatcttcgtgaagaccctgactggtaaga
ccatcactctcgaggtggagccgagtgacaccattgagaatgtcaagg
caaagatccaagacaaggaaggcatccctcctgaccagcagaggttga
tctttgctgggaaacagctggaagatggacgcaccctgtctgactaca
acatccagaaagagtccaccctgcacctggtgctccgtcttagaggtg
ggatgcagatcttcgtgaagaccctgactggtaagaccatcactctcg
aagtggagccgagtgacaccattgagaatgtcaaggcaaagatccaag
acaaggaaggcatccctcctgaccagcagaggttgatctttgctggga
aacagctggaagatggacgcaccctgtctgactacaacatccagaaag
agtccaccctgcacctggtgctccgtcttagaggtgggatgcagatct
tcgtgaagaccctgactggtaagaccatcactctcgaagtggagccga
gtgacaccattgagaatgtcaaggcaaagatccaagacaaggaaggca
tccctcctgaccagcagaggttgatctttgctgggaaacagctggaag
atggacgcaccctgtctgactacaacatccagaaagagtccaccctgc
acctggtgctccgtctcagaggtgggatgcaaatcttcgtgaagaccc
tgactggtaagaccatcaccctcgaggtggagcccagtgacaccatcg
agaatgtcaaggcaaagatccaagataaggaaggcatccctcctgatc
agcagaggttgatctttgctgggaaacagctggaagatggacgcaccc
tgtctgactacaacatccagaaagagtccactctgcacttggtcctgc
gcttgagggggggtgtctaagtttccccttttaaggtttcaacaaatt
tcattgcactttcctttcaataaagttgttgcattcccaaaaaaaaaa
aa

In an embodiment of the invention HPRT1 comprises the nucleotide sequence or nucleotides 168-824 thereof (see Genbank accession no. NM_{—}000194):

HPRT1 >nucfasta||NM_000194 NM_000194 2
(SEQ ID NO: 17)
ggcggggcctgcttctcctcagcttcaggcggctgcgacgagccctca
ggcgaacctctcggctttcccgcgcggcgccgcctcttgctgcgcctc
cgcctcctcctctgctccgccaccggcttcctcctcctgagcagtcag
cccgcgcgccggccggctccgttatggcgacccgcagccctggcgtcg
tgattagtgatgatgaaccaggttatgaccttgatttattttgcatac
ctaatcattatgctgaggatttggaaagggtgtttattcctcatggac
taattatggacaggactgaacgtcttgctcgagatgtgatgaaggaga
tgggaggccatcacattgtagccctctgtgtgctcaaggggggctata
aattctttgctgacctgctggattacatcaaagcactgaatagaaata
gtgatagatccattcctatgactgtagattttatcagactgaagagct
attgtaatgaccagtcaacaggggacataaaagtaattggtggagatg
atctctcaactttaactggaaagaatgtcttgattgtggaagatataa
ttgacactggcaaaacaatgcagactttgctttccttggtcaggcagt
ataatccaaagatggtcaaggtcgcaagcttgctggtgaaaaggaccc
cacgaagtgttggatataagccagactttgttggatttgaaattccag
acaagtttgttgtaggatatgcccttgactataatgaatacttcaggg
atttgaatcatgtttgtgtcattagtgaaactggaaaagcaaaataca
aagcctaagatgagagttcaagttgagtttggaaacatctggagtcct
attgacatcgccagtaaaattatcaatgttctagttctgtggccatct
gcttagtagagctttttgcatgtatcttctaagaattttatctgtttt
gtactttagaaatgtcagttgctgcattcctaaactgtttatttgcac
tatgagcctatagactatcagttccctttgggcggattgttgtttaac
ttgtaaatgaaaaaattctcttaaaccacagcactattgagtgaaaca
ttgaactcatatctgtaagaaataaagagaagatatattagtttttta
attggtattttaatttttatatatgcaggaaagaatagaagtgattga
atattgttaattataccaccgtgtgttagaaaagtaagaagcagtcaa
ttttcacatcaaagacagcatctaagaagttttgttctgtcctggaat
tattttagtagtgtttcagtaatgttgactgtattttccaacttgttc
aaattattaccagtgaatctttgtcagcagttcccttttaaatgcaaa
tcaataaattcccaaaaatttaaaaaaaaaaaaaaaaaaaaaa

In an embodiment of the invention CYC1 comprises the nucleotide sequence or nucleotides 44-1021 thereof (see Genbank accession no. NM_{—}001916):

CYC1 >nucfasta||NM_001916 NM_001916 3
(SEQ ID NO: 18)
cccaggggccgacgggagtggcggccgcgcggaggaggccaagatggc
ggcagctgcggcttcgcttcgcggggtagtgttgggcccgcggggcgc
ggggctcccgggcgcgcgtgcccggggtctgctgtgcagcgcgcgtcc
cgggcagctcccgctacggacacctcaggcagtggccttgtcgtcgaa
gtctggcctttcccgaggccggaaagtgatgctgtcagcgctgggcat
gctggcggcagggggtgcggggctggccgtggctctgcattcggctgt
gagtgccagtgacctggagctgcacccccccagctatccgtggtctca
ccgtggcctcctctcttccttggaccacaccagcatccggaggggttt
ccaggtatataagcaggtgtgcgcctcctgccacagcatggacttcgt
ggcctaccgccacctggtgggcgtgtgctacacggaggatgaagctaa
ggagctggctgcggaggtggaggttcaagacggccccaatgaagatgg
ggagatgttcatgcggccagggaagctgttcgactatttcccaaaacc
ataccccaacagtgaggctgctcgagctgccaacaacggagcattgcc
ccctgacctcagctacatcgtgcgagctaggcatggtggtgaggacta
cgtcttctccctgctcacgggctactgcgagccacccaccggggtgtc
actgcgggaaggtctctacttcaacccctactttcctggccaggccat
tgccatggcccctcccatctacacagatgtcttagagtttgacgatgg
caccccagctaccatgtcccagatagccaaggatgtgtgcaccttcct
gcgctgggcatctgagccagagcacgaccatcgaaaacgcatggggct
caagatgttgatgatgatggctctgctggtgcccctggtctacaccat
aaagcggcacaagtggtcagtcctgaagagtcggaagctggcatatcg
gccgcccaagtgaccctgtccagtgtctgcttgccatcctgccagaac
aggccctcaagcccaagagccatcccaggcctgttcaggcctcagcta
agcctctcttcatctggaagaagaggcaagggggcaggagaccaggct
ctagctctgggccctccttcagcccccatcatgggaataaattaattt
tctcaatgtaaaaaaaaaaaaaaaaaaaaaaaaaa

In an embodiment of the invention HMBS comprises the nucleotide sequence or nucleotides 158-1243 thereof (see Genbank accession no. NM_{—}000190):

HMBS >nucfasta||NM_000190 NM_000190 3
(SEQ ID NO: 19)
ccggaagtgacgcgaggctctgcggagaccaggagtcagactgtagga
cgacctcgggtcccacgtgtccccggtactcgccggccggagcccccg
gcttcccggggccgggggaccttagcggcacccacacacagcctactt
tccaagcggagccatgtctggtaacggcaatgcggctgcaacggcgga
agaaaacagcccaaagatgagagtgattcgcgtgggtacccgcaagag
ccagcttgctcgcatacagacggacagtgtggtggcaacattgaaagc
ctcgtaccctggcctgcagtttgaaatcattgctatgtccaccacagg
ggacaagattcttgatactgcactctctaagattggagagaaaagcct
gtttaccaaggagcttgaacatgccctggagaagaacgaagtggacct
ggttgttcactccttgaaggacctgcccactgtgcttcctcctggctt
caccatcggagccatctgcaagcgggaaaaccctcatgatgctgttgt
ctttcacccaaaatttgttgggaagaccctagaaaccctgccagagaa
gagtgtggtgggaaccagctccctgcgaagagcagcccagctgcagag
aaagttcccgcatctggagttcaggagtattcggggaaacctcaacac
ccggcttcggaagctggacgagcagcaggagttcagtgccatcatcct
ggcaacagctggcctgcagcgcatgggctggcacaaccgggtggggca
gatcctgcaccctgaggaatgcatgtatgctgtgggccagggggcctt
gggcgtggaagtgcgagccaaggaccaggacatcttggatctggtggg
cgtgctgcacgatcccgagactctgcttcgctgcatcgctgaaagggc
cttcctgaggcacctggaaggaggctgcagtgtgccagtagccgtgca
tacagctatgaaggatgggcaactgtacctgactggaggagtctggag
tctagacggctcagatagcatacaagagaccatgcaggctaccatcca
tgtccctgcccagcatgaagatggccctgaggatgacccacagttggt
aggcatcactgctcgtaacattccacgagggccccagttggctgccca
gaacttgggcatcagcctggccaacttgttgctgagcaaaggagccaa
aaacatcctggatgttgcacggcagcttaacgatgcccattaactggt
ttgtggggcacagatgcctgggttgctgctgtccagtgcctacatccc
gggcctcagtgccccattctcactgctatctggggagtgattaccccg
ggagactgaactgcagggttcaagccttccagggatttgcctcacctt
ggggccttgatgactgccttgcctcctcagtatgtgggggcttcatct
ctttagagaagtccaagcaacagcctttgaatgtaaccaatcctacta
ataaaccagttctgaaggtgtaaaaaaaaaaaaaaaaa

In an embodiment of the invention SDHA comprises the nucleotide sequence or nucleotides 116-2110 thereof (see Genbank accession no. NM_{—}004168):

SDHA>nucfasta||NM_004168 NM_004168 2
(SEQ ID NO: 20)
tccggcgtggtgcgcaggcgcggtatcccccctcccccgccagctcga
ccccggtgtggtgcgcaggcgcagtctgcgcagggactggcgggactg
cgcggcggcaacagcagacatgtcgggggtccggggcctgtcgcggct
gctgagcgctcggcgcctggcgctggccaaggcgtggccaacagtgtt
gcaaacaggaacccgaggttttcacttcactgttgatgggaacaagag
ggcatctgctaaagtttcagattccatttctgctcagtatccagtagt
ggatcatgaatttgatgcagtggtggtaggcgctggaggggcaggctt
gcgagctgcatttggcctttctgaggcagggtttaatacagcatgtgt
taccaagctgtttcctaccaggtcacacactgctgcagcacagggagg
aatcaatgctgctctggggaacatggaggaggacaactggaggtggca
tttctacgacaccgtgaagggctccgactggctgggggaccaggatgc
catccactacatgacggagcaggcccccgccgccgtggtcgagctaga
aaattatggcatgccgtttagcagaactgaagatgggaagatttatca
gcgtgcatttggtggacagagcctcaagtttggaaagggcgggcaggc
ccatcggtgctgctgtgtggctgatcggactggccactcgctattgca
caccttatatggaaggtctctgcgatatgataccagctattttgtgga
gtattttgccttggatctcctgatggagaatggggagtgccgtggtgt
catcgcactgtgcatagaggacgggtccatccatcgcataagagcaaa
gaacactgttgttgccacaggaggctacgggcgcacctacttcagctg
cacgtctgcccacaccagcactggcgacggcacggccatgatcaccag
ggcaggccttccttgccaggacctagagtttgttcagttccaccctac
aggcatatatggtgctggttgtctcattacggaaggatgtcgtggaga
gggaggcattctcattaacagtcaaggcgaaaggtttatggagcgata
cgcccctgtcgcgaaggacctggcgtctagagatgtggtgtctcggtc
catgactctggagatccgagaaggaagaggctgtggccctgagaaaga
tcacgtctacctgcagctgcaccacctacctccagagcagctggccac
gcgcctgcctggcatttcagagacagccatgatcttcgctggcgtgga
cgtcacgaaggagccgatccctgtcctccccaccgtgcattataacat
gggcggcattcccaccaactacaaggggcaggtcctgaggcacgtgaa
tggccaggatcagattgtgcccggcctgtacgcctgtggggaggccgc
ctgtgcctcggtacatggtgccaaccgcctcggggcaaactcgctctt
ggacctggttgtctttggtcgggcatgtgccctgagcatcgaagagtc
atgcaggcctggagataaagtccctccaattaaaccaaacgctgggga
agaatctgtcatgaatcttgacaaattgagatttgctgatggaagcat
aagaacatcggaactgcgactcagcatgcagaagtcaatgcaaaatca
tgctgccgtgttccgtgtgggaagcgtgttgcaagaaggttgtgggaa
aatcagcaagctctatggagacctaaagcacctgaagacgttcgaccg
gggaatggtctggaacacggacctggtggagaccctggagctgcagaa
cctgatgctgtgtgcgctgcagaccatctacggagcagaggcacggaa
ggagtcacggggcgcgcatgccagggaagactacaaggtgcggattga
tgagtacgattactccaagcccatccaggggcaacagaagaagccctt
tgaggagcactggaggaagcacaccctgtcctatgtggacgttggcac
tgggaaggtcactctggaatatagacccgtgatcgacaaaactttgaa
cgaggctgactgtgccaccgtcccgccagccattcgctcctactgatg
agacaagatgtggtgatgacagaatcagcttttgtaattatgtataat
agctcatgcatgtgtccatgtcataactgtcttcatacgcttctgcac
tctggggaagaaggagtacattgaagggagattggcacctagtggctg
ggagcttgccaggaacccagtggccagggagcgtggcacttacctttg
tcccttgcttcattcttgtgagatgataaaactgggcacagctcttaa
ataaaatataaatgaacaaactttcttttatttccaaaaaaaaaaaaa
aaaaa

In an embodiment of the invention ACTB comprises the nucleotide sequence or nucleotides 85-1212 thereof (see Genbank accession no. NM_{—}001101):

ACTB >nucfasta||NM_001101 NM_001101 3
(SEQ ID NO: 21)
accgccgagaccgcgtccgccccgcgagcacagagcctcgcctttgcc
gatccgccgcccgtccacacccgccgccagctcaccatggatgatgat
atcgccgcgctcgtcgtcgacaacggctccggcatgtgcaaggccggc
ttcgcgggcgacgatgccccccgggccgtcttcccctccatcgtgggg
cgccccaggcaccagggcgtgacggtgggcatgggtcagaaggattcc
tatgtgggcgacgaggcccagagcaagagaggcatcctcaccctgaag
taccccatcgagcacggcatcgtcaccaactgggacgacatggagaaa
atctggcaccacaccttctacaatgagctgcgtgtggctcccgaggag
caccccgtgctgctgaccgaggcccccctgaaccccaaggccaaccgc
gagaagatgacccagatcatgtttgagaccttcaacaccccagccatg
tacgttgctatccaggctgtgctatccctgtacgcctctggccgtacc
actggcatcgtgatggactccggtgacggggtcacccacactgtgccc
atctacgaggggtatgccctcccccatgccatcctgcgtctggacctg
gctggccgggacctgactgactacctcatgaagatcctcaccgagcgc
ggctacagcttcaccaccacggccgagcgggaaatcgtgcgtgacatt
aaggagaagctgtgctacgtcgccctggacttcgagcaagagatggcc
acggctgcttccagctcctccctggagaagagctacgagctgcctgac
ggccaggtcatcaccattggcaatgagcggttccgctgccctgaggca
ctcttccagccttccttcctgggcatggagtcctgtggcatccacgaa
actaccttcaactccatcatgaagtgtgacgtggacatccgcaaagac
ctgtacgccaacacagtgctgtctggcggcaccaccatgtaccctggc
attgccgacaggatgcagaaggagatcactgccctggcacccagcaca
atgaagatcaagatcattgctcctcctgagcgcaagtactccgtgtgg
atcggcggctccatcctggcctcgctgtccaccttccagcagatgtgg
atcagcaagcaggagtatgacgagtccggcccctccatcgtccaccgc
aaatgcttctaggcggactatgacttagttgcgttacaccctttcttg
acaaaacctaacttgcgcagaaaacaagatgagattggcatggcttta
tttgttttttttgttttgttttggttttttttttttttttggcttgac
tcaggatttaaaaactggaacggtgaaggtgacagcagtcggttggag
cgagcatcccccaaagttcacaatgtggccgaggactttgattgcaca
ttgttgtttttttaatagtcattccaaatatgagatgcgttgttacag
gaagtcccttgccatcctaaaagccaccccacttctctctaaggagaa
tggcccagtcctctcccaagtccacacaggggaggtgatagcattgct
ttcgtgtaaattatgtaatgcaaaatttttttaatcttcgccttaata
cttttttattttgttttattttgaatgatgagccttcgtgccccccct
tcccccttttttgtcccccaacttgagatgtatgaaggcttttggtct
ccctgggagtgggtggaggcagccagggcttacctgtacactgacttg
agaccagttgaataaaagtgcacaccttaaaaatgaaaaaaaaaaaaa
aaaaaaaaaaaaaaaaaaaaaaaaaaaa

In an embodiment of the invention GUSB comprises the nucleotide sequence or nucleotides 132-2087 thereof (see Genbank accession no. NM_{—}000181):

GUSB >nucfasta||NM_000181 NM_000181 3
(SEQ ID NO: 22)
gtcctcaaccaagatggcgcggatggcttcaggcgcatcacgacaccg
gcgcgtcacgcgacccgccctacgggcacctcccgcgcttttcttagc
gccgcagacggtggccgagcgggggaccgggaagcatggcccgggggt
cggcggttgcctgggcggcgctcgggccgttgttgtggggctgcgcgc
tggggctgcagggcgggatgctgtacccccaggagagcccgtcgcggg
agtgcaaggagctggacggcctctggagcttccgcgccgacttctctg
acaaccgacgccggggcttcgaggagcagtggtaccggcggccgctgt
gggagtcaggccccaccgtggacatgccagttccctccagcttcaatg
acatcagccaggactggcgtctgcggcattttgtcggctgggtgtggt
acgaacgggaggtgatcctgccggagcgatggacccaggacctgcgca
caagagtggtgctgaggattggcagtgcccattcctatgccatcgtgt
gggtgaatggggtcgacacgctagagcatgaggggggctacctcccct
tcgaggccgacatcagcaacctggtccaggtggggcccctgccctccc
ggctccgaatcactatcgccatcaacaacacactcacccccaccaccc
tgccaccagggaccatccaatacctgactgacacctccaagtatccca
agggttactttgtccagaacacatattttgactttttcaactacgctg
gactgcagcggtctgtacttctgtacacgacacccaccacctacatcg
atgacatcaccgtcaccaccagcgtggagcaagacagtgggctggtga
attaccagatctctgtcaagggcagtaacccgttcaagttggaagtgc
gtcttttggatgcagaaaacaaagtcgtggcgaatgggactgggaccc
agggccaacttaaggtgccaggtgtcagcctctggtggccgtacctga
tgcacgaacgccctgcctatctgtattcattggaggtgcagctgactg
cacagacgtcactggggcctgtgtctgacttctacacactccctgtgg
ggatccgcactgtggctgtcaccaagagccagttcctcatcaatggga
aacctttctatttccacggtgtcaacaagcatgaggatgcggacatcc
gagggaagggcttcgactggccgctgctggtgaaggacttcaacctgc
ttcgctggcttggtgccaacgctttccgtaccagccactacccctatg
cagaggaagtgatgcagatgtgtgaccgctatgggattgtggtcatcg
atgagtgtcccggcgtgggcctggcgctgccgcagttcttcaacaacg
tttctctgcatcaccacatgcaggtgatggaagaagtggtgcgtaggg
acaagaaccaccccgcggtcgtgatgtggtctgtggccaacgagcctg
cgtcccacctagaatctgctggctactacttgaagatggtgatcgctc
acaccaaatccttggacccctcccggcctgtgacctttgtgagcaact
ctaactatgcagcagacaagggggctccgtatgtggatgtgatctgtt
tgaacagctactactcttggtatcacgactacgggcacctggagttga
ttcagctgcagctggccacccagtttgagaactggtataagaagtatc
agaagcccattattcagagcgagtatggagcagaaacgattgcagggt
ttcaccaggatccacctctgatgttcactgaagagtaccagaaaagtc
tgctagagcagtaccatctgggtctggatcaaaaacgcagaaaatacg
tggttggagagctcatttggaattttgccgatttcatgactgaacagt
caccgacgagagtgctggggaataaaaaggggatcttcactcggcaga
gacaaccaaaaagtgcagcgttccttttgcgagagagatactggaaga
ttgccaatgaaaccaggtatccccactcagtagccaagtcacaatgtt
tggaaaacagcctgtttacttgagcaagactgataccacctgcgtgtc
ccttcctccccgagtcagggcgacttccacagcagcagaacaagtgcc
tcctggactgttcacggcagaccagaacgtttctggcctgggttttgt
ggtcatctattctagcagggaacactaaaggtggaaataaaagatttt
ctattatggaaataaagagttggcatgaaagtggctactgaaaaaaaa
aaaaaaaaaaaaaaaaa

In an embodiment of the invention TOP1 comprises the nucleotide sequence or nucleotides 247-2544 thereof (see Genbank accession no. NM_{—}003286):

TOP1 >nucfasta|| NM_003286 NM_003286 2
(SEQ ID NO: 23)
caaatgcgaacttaggctgttacacaactgctggggtctgttctcgcc
gcccgcccggcagtcaggcagcgtcgccgccgtggtagcagcctcagc
cgtttctggagtctcgggcccacagtcaccgccgcttacctgcgcctc
ctcgagcctccggagtccccgtccgcccgcacaggccggttcgccgtc
tgcgtctcccccacgccgcctcgcctgccgccgcgctcgtccctccgg
gccgacatgagtggggaccacctccacaacgattcccagatcgaagcg
gatttccgattgaatgattctcataaacacaaagataaacacaaagat
cgagaacaccggcacaaagaacacaagaaggagaaggaccgggaaaag
tccaagcatagcaacagtgaacataaagattctgaaaagaaacacaaa
gagaaggagaagaccaaacacaaagatggaagctcagaaaagcataaa
gacaaacataaagacagagacaaggaaaaacgaaaagaggaaaaggtt
cgagcctctggggatgcaaaaataaagaaggagaaggaaaatggcttc
tctagtccaccacaaattaaagatgaacctgaagatgatggctatttt
gttcctcctaaagaggatataaagccattaaagagacctcgagatgag
gatgatgctgattataaacctaagaaaattaaaacagaagataccaag
aaggagaagaaaagaaaactagaagaagaagaggatggtaaattgaaa
aaacccaagaataaagataaagataaaaaagttcctgagccagataac
aagaaaaagaagccgaagaaagaagaggaacagaagtggaaatggtgg
gaagaagagcgctatcctgaaggcatcaagtggaaattcctagaacat
aaaggtccagtatttgccccaccatatgagcctcttccagagaatgtc
aagttttattatgatggtaaagtcatgaagctgagccccaaagcagag
gaagtagctacgttctttgcaaaaatgctcgaccatgaatatactacc
aaggaaatatttaggaaaaatttctttaaagactggagaaaggaaatg
actaatgaagagaagaatattatcaccaacctaagcaaatgtgatttt
acccagatgagccagtatttcaaagcccagacggaagctcggaaacag
atgagcaaggaagagaaactgaaaatcaaagaggagaatgaaaaatta
ctgaaagaatatggattctgtattatggataaccacaaagagaggatt
gctaacttcaagatagagcctcctggacttttccgtggccgcggcaac
caccccaagatgggcatgctgaagagacgaatcatgcccgaggatata
atcatcaactgtagcaaagatgccaaggttccttctcctcctccagga
cataagtggaaagaagtccggcatgataacaaggttacttggctggtt
tcctggacagagaacatccaaggttccattaaatacatcatgcttaac
cctagttcacgaatcaagggtgagaaggactggcagaaatacgagact
gctcggcggctgaaaaaatgtgtggacaagatccggaaccagtatcga
gaagactggaagtccaaagagatgaaagtccggcagagagctgtagcc
ctgtacttcatcgacaagcttgctctgagagcaggcaatgaaaaggag
gaaggagaaacagcggacactgtgggctgctgctcacttcgtgtggag
cacatcaatctacacccagagttggatggtcaggaatatgtggtagag
tttgacttcctcgggaaggactccatcagatactataacaaggtccct
gttgagaaacgagtttttaagaacctacaactatttatggagaacaag
cagcccgaggatgatctttttgatagactcaatactggtattctgaat
aagcatcttcaggatctcatggagggcttgacagccaaggtattccgt
acatacaatgcctccatcacgctacagcagcagctaaaagaactgaca
gccccggatgagaacatcccagcgaagatcctttcttataaccgtgcc
aatcgagctgttgcaattctttgtaaccatcagagggcaccaccaaaa
acttttgagaagtctatgatgaacttgcaaactaagattgatgccaag
aaggaacagctagcagatgcccggagagacctgaaaagtgctaaggct
gatgccaaggtcatgaaggatgcaaagacgaagaaggtagtagagtca
aagaagaaggctgttcagagactggaggaacagttgatgaagctggaa
gttcaagccacagaccgagaggaaaataaacagattgccctgggaacc
tccaaactcaattatctggaccctaggatcacagtggcttggtgcaag
aagtggggtgtcccaattgagaagatttacaacaaaacccagcgggag
aagtttgcctgggccattgacatggctgatgaagactatgagttttag
ccagtctcaagaggcagagttctgtgaagaggaacagtgtggtttggg
aaagatggataaactgagcctcacttgccctcgtgcctgggggagaga
ggcagcaagtcttaacaaaccaacatctttgcgaaaagataaacctgg
agatattataagggagagctgagccagttgtcctatggacaacttatt
taaaaatatttcagatatcaaaattctagctgtatgatttgttttgaa
ttttgtttttattttcaagagggcaagtggatgggaatttgtcagcgt
tctaccaggcaaattcactgtttcactgaaatgtttggattctcttag
ctactgtatgcaaagtccgattatattggtgcgtttttacagttaggg
ttttgcaataacttctatattttaatagaaataaattcctaaactccc
ttccctctctcccatttcaggaatttaaaattaagtagaacaaaaaac
ccagcgcacctgttagagtcgtcactctctattgtcatggggatcaat
tttcattaaacttgaagcagtcgtggctttggcagtgttttggttcag
acacctgttcacagaaaaagcatgatgggaaaatatttcctgacttga
gtgttcctttttaaatgtgaatttttatttctttttaattattttaaa
atatttaaacctttttcttgatcttaaagatcgtgtagattggggttg
gggagggatgaagggcgagtgaatctaaggataatgaaataatcagtg
actgaaaccattttcccatcatcctttgttctgagcattcgctgtacc
ctttaagatatccatctttttctttttaaccctaatctttcacttgaa
agattttattgtataaaaagtttcacaggtcaataaacttagaggaaa
atgagtatttggtccaaaaaaaggaaaaataatcaagattttagggct
tttattttttcttttgtaattgtgtaaaaaatggaaaaaaacataaaa
agcagaattttaatgtgaagacattttttgctataatcattagtttta
gaggcattgttagtttagtgtgtgtgcagagtccatttcccacatctt
tcctcaagtatcttctatttttatcatgaattcccttttaatcaactg
taggttatttaaaataaattcctacaacttaatggaaa

In an embodiment of the invention B2M comprises the nucleotide sequence or nucleotides 61-420 thereof (see Genbank accession no. NM_{—}004048):

B2M >nucfasta|| NM_004048 NM_004048 2
(SEQ ID NO: 24)
aatataagtggaggcgtcgcgctggcgggcattcctgaagctgacagc
attcgggccgagatgtctcgctccgtggccttagctgtgctcgcgcta
ctctctctttctggcctggaggctatccagcgtactccaaagattcag
gtttactcacgtcatccagcagagaatggaaagtcaaatttcctgaat
tgctatgtgtctgggtttcatccatccgacattgaagttgacttactg
aagaatggagagagaattgaaaaagtggagcattcagacttgtctttc
agcaaggactggtctttctatctcttgtactacactgaattcaccccc
actgaaaaagatgagtatgcctgccgtgtgaaccatgtgactttgtca
cagcccaagatagttaagtgggatcgagacatgtaagcagcatcatgg
aggtttgaagatgccgcatttggattggatgaattccaaattctgctt
gcttgctttttaatattgatatgcttatacacttacactttatgcaca
aaatgtagggttataataatgttaacatggacatgatcttctttataa
ttctactttgagtgctgtctccatgtttgatgtatctgagcaggttgc
tccacaggtagctctaggagggctggcaacttagaggtggggagcaga
gaattctcttatccaacatcaacatcttggtcagatttgaactcttca
atctcttgcactcaaagcttgttaagatagttaagcgtgcataagtta
acttccaatttacatactctgcttagaatttgggggaaaatttagaaa
tataattgacaggattattggaaatttgttataatgaatgaaacattt
tgtcatataagattcatatttacttcttatacatttgataaagtaagg
catggttgtggttaatctggtttatttttgttccacaagttaaataaa
tcataaaacttgatgtgttatctctta

In an embodiment of the invention GAPDH comprises the nucleotide sequence or nucleotides 103-1110 thereof (see Genbank accession no. NM_{—}002046):

GAPDH >nucfasta||NM_002046 NM_002046 3
(SEQ ID NO: 25)
aaattgagccogcagcctcccgcttcgctctctgctcctcctgttcga
cagtcagccgcatcttcttttgcgtcgccagccgagccacatcgctca
gacaccatggggaaggtgaaggtcggagtcaacggatttggtcgtatt
gggcgcctggtcaccagggctgcttttaactctggtaaagtggatatt
gttgccatcaatgaccccttcattgacctcaactacatggtttacatg
ttccaatatgattccacccatggcaaattccatggcaccgtcaaggct
gagaacgggaagcttgtcatcaatggaaatcccatcaccatcttccag
gagcgagatccctccaaaatcaagtggggcgatgctggcgctgagtac
gtcgtggagtccactggcgtcttcaccaccatggagaaggctggggct
catttgcagggsggagccaaaagggtcatcatctctgccccctctgct
gatgcccccatgttcgtcatgggtgtgaaccatgagaagtatgacaac
agcctcaagatcatcagcaatgcctcctgcaccaccaactgcttagca
cccctggccaaggtcatccatgacaactttggtatcgtggaaggactc
atgaccacagtccatgccatcactgccacccagaagactgtggatggc
ccctccgggaaactgtggcgtgatggccgcggggctctccagaacatc
atccctgcctctactggcgctgccaaggctgtgggcaaggtcatccct
gagctgaacgggaagctcactggcatggccttccgtgtccccactgcc
aacgtgtcagtggtggacctgacctgccgtctagaaaaacctgccaaa
tatgatgacatcaagaaggtggtgaagcaggcgtcggagggccccctc
aagggcatcctgggctacactgagcaccaggtggtctcctctgacttc
aacagcgacacccactcctccacctttgacgctggggctggcattgcc
ctcaacgaccactttgtcaagctcatttcctggtatgacaacgaattt
ggctacagcaacagggtggtggacctcatggcccacatggcctocaag
gagtaagacccctggaccaccagccccagcaagagcacaagaggaaga
gagagaccctcactgctggggagtccctgccacactcagtcccccacc
acactgaatctcccctcctcacagttgccatgtagaccccttgaagag
gggaggggcctagggagccgcaccttgtcatgtaccatcaataaagta
ccctgtgctcaacc

In an embodiment of the invention, the reference gene used in methods of the present invention comprises a nucleotide sequence selected from SEQ ID NOs: 14-25 or a polypeptide coding sequence thereof or a variant of the gene that comprise at least 80% (e.g., 90%, 92%, 95%, 98% or 99%) identity to a reference sequence selected from SEQ ID NOs: 14-25 when the comparison is performed by a BLAST algorithm (e.g., BLASTN) wherein the parameters of the algorithm are selected to give the largest match between the respective sequences over the entire length of the respective reference sequences.

“Cq” is the fractional cycle number, of a real time polymerase chain reaction amplification of a given target gene, wherein acceleration of amplification in the reaction is at a maximum; or, wherein logarithmic increases in amplification over time can no longer be sustained; which may be expressed relative to that of one or more reference genes.

A fractional cycle number of at or below about 1.83 to at or below about 2.03, in an embodiment of the invention, includes fractional cycle numbers at or below values up to 5% (e.g., 1%, 2%, 3%, 4% or 5%) less than 1.83 and/or at or below values up to 5% (e.g., 1%, 2%, 3%, 4% or 5%) higher than of 2.03. At or below “about 1.83 to about 2.03” as used herein includes values at or below 1.83 (or − up to 5% thereof) or at or below 2.03 (or + up to 5% thereof) as well as any value at or below values that are between these limits, e.g., at or below 1.84, at or below 1.85, at or below 1.86, at or below 1.87, at or below 1.88, at or below 1.89, at or below 1.90, at or below 1.91, at or below 1.92, at or below 1.93, at or below 1.94, at or below 1.95, at or below 1.96, at or below 1.97, at or below 1.98, at or below 1.99, at or below 2.00, at or below 2.01; or at or below 2.02 (e.g., at or below about 1.7, 1.6, 1.5, 1.4, 1.3, 1.2, 1.1, 1.0, 0.9, 0.8, 0.7, 0.6 or 0.5). Accordingly, methods described herein in terms of fractional cycle numbers at or below values in this range should also be understood to include embodiments wherein these methods are described in terms of fractional cycle numbers having values at or below any of these single values.

The methods of the present invention may further comprise evaluation of KRAS expression in tumors (in association with evaluation of IGF1 expression as discussed herein) (e.g., ovarian tumors) to determine whether a tumor cell is sensitive to IGF1R inhibitor therapy. Specifically, KRAS expression levels can be measured wherein, when KRAS levels are observed to be low, then, in an embodiment of the invention, the tumor cell analyzed is determined to be sensitive to IGF1R inhibitor therapy. Identification of high expression of KRAS or the presence of an activating KRAS mutation (e.g., Gly12Asp, Gly12Ala, Gly12Val, Gly12Ser, Gly12Arg or Gly13Asp) in a tumor cell indicates, in an embodiment of the invention, that the tumor cell is relatively insensitive to IGF1R inhibitor therapy. See Scartozzi et al., Int J Cancer. 127(8):1941-1947 (2010).

A positive clinical outcome, in an embodiment of the invention, refers to shrinkage of a tumor or an increase in progression free survival or overall survival or an improvement in the signs and/or symptoms of the tumor in a subject, e.g., relative to that of the subject pre-treatment or another subject with a similar disease not having treatment.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

Reverse transcription polymerase chain reaction (RT-PCR) is a variant of polymerase chain reaction (PCR) wherein RNA is reverse transcribed into its DNA complement (complementary DNA, or cDNA) using the enzyme reverse transcriptase, and the resulting cDNA is amplified using PCR. These two steps may occur in a single tube or in separate tubes.

A polypeptide or protein comprises two or more amino acids.

The term “isolated protein”, “isolated polypeptide” or “isolated antibody” is a protein, polypeptide or antibody was purified to any degree.

A “polynucleotide”, “nucleic acid” or “nucleic acid molecule” includes double-stranded and single-stranded DNA and RNA.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

An amino acid sequence comprises two or more amino acids.

A “coding sequence” or a sequence “encoding” an expression product, such as an RNA or polypeptide, is a nucleotide sequence that, when expressed, results in production of the product.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

A coding sequence, such as a reporter gene, is “under the control of”, “functionally associated with” or “operably linked to” a transcriptional and translational control sequence, such as a promoter, e.g., in an isolated host cell, when the sequences direct RNA polymerase mediated transcription of the coding sequence into RNA, e.g., mRNA, which then may be trans-RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The terms “vector”, “cloning vector” and “expression vector” mean the vehicle (e.g., a plasmid) by which a DNA or RNA sequence can be introduced into a host cell, so as to transform the host and, optionally, promote expression and/or replication of the introduced sequence.

A cell, such as a tumor cell, is sensitive to an IGF1R inhibitor is its growth, survival and/or metastasis is inhibited by the inhibitor.

The following references regarding the BLAST algorithm are herein incorporated by reference: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M., et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

### Therapeutic Methods

The present invention provides a method for treating an IGF1-expressing tumor (e.g., wherein the tumor also expresses IGF1R) in a subject; or for selecting a subject (e.g., human) for IGF1R inhibitor therapy (e.g., dalotuzumab) for a tumor (e.g., a subject having a tumor that is sensitive to IGF1R inhibitor therapy and/or likely to experience a positive clinical outcome upon treatment with an IGF1R inhibitor); or for selecting a therapy in a subject (e.g., a subject having a tumor that is sensitive to IGF1R inhibitor therapy and/or likely to experience a positive clinical outcome upon treatment with an IGF1R inhibitor) with a tumor; based on the expression level of IGF1 mRNA in cells of the subject's tumor. The method comprises treating tumors having cells that have been observed to express IGF1 mRNA at least at a certain threshold level (e.g., prior to IGF1R inhibitor-based therapy). In an embodiment of the invention, the threshold level is expressed in terms of IGF1 mRNA expression levels in said tumor cells, measured using RT-PCR and real time PCR, relative to or normalized against mRNA expression levels, measured by RT-PCR and real time PCR, of any of 12 reference genes: CYC1, HMBS, TOP1, SDHA, GUSB, PUM1, HPRT1, ACTB, UBC, B2M, GAPDH, and TUBB2A (e.g., any of SEQ ID NOs: 14-25 or a cDNA thereof) in said tumor cells or tissue. In an embodiment of the invention, any 1, 2, 3, 4, 5, 6, 7, 8, 9, 10, 11 or 12 reference gene mRNA levels are used in the comparison to IGF1 mRNA expression levels. The expression levels of IGF1 mRNA normalized to that of the reference genes is, in an embodiment of the invention, expressed in terms of comparative quantification (Cq).

In an embodiment of the invention, tumor cells treated using methods of the present invention express IGF1 as well as IGF1R, wherein, for example, growth and/or survival and/or metastasis of the tumor cells is mediated, at least in part, by the activity and/or expression level of IGF1 and/or IGF1R. In such an embodiment, tumor growth, survival and/or metastasis is inhibited by an IGF1R inhibitor.

Tumor IGF1 RNA expression levels are measured prior to a given course or dose of IGF1R inhibitor therapy in the subject. In an embodiment of the invention, IGF1RNA expression is measured before any IGF1R inhibitor therapy has commenced in the subject. In an embodiment of the invention, IGF1 RNA expression levels can be measured after one or more courses of IGF1R inhibitor therapy have started but before one or more further course of IGF1R inhibitor therapy begin.

In an embodiment of the invention, reverse transcription polymerase chain reaction and real time polymerase chain reaction are used to determine the IGF1 mRNA expression level. Various methods for performing RT-PCR are known in the art including one-step and two-step RT-PCR—each of which may be used to quantitate IGF1 mRNA. Suitable primers may be designed for doing so; for example, primers comprising the following nucleotide sequences may be used: forward primer having the nucleotide sequence of SEQ ID NO: 3, 4, 5, 6 or 7) and reverse primer having the nucleotide sequence of SEQ ID NO: 8, 9, 10, 11 or 12. The present invention includes embodiments wherein such primer pairs are used to quantitate IGF1. The real time PCR data characterizing IGF1 mRNA expression levels may be normalized against the expression levels of one or more reference genes (e.g., whose expression level was also determined by real time PCR) whose expression is known not to increase or decrease significantly in tumor cells relative to normal cells. For example, in an embodiment of the invention, suitable reference genes include any one or more of CYC1, HMBS, TOP1, SDHA, GUSB, PUM1, HPRT1, ACTB, UBC, B2M, GAPDH, and TUBB2A (e.g., any of SEQ ID NOs: 14-25 or a cDNA thereof). The IGF1 mRNA expression levels, normalized against that of the reference gene(s), can be used to generate a Cq value wherein the real time PCR expression level data is analyzed using the absolute quantification second derivative maximum method. Practitioners of ordinary skill in the art understand how to arrive at the Cq values for IGF1 mRNA expression levels from tumor cells.

A convenient and effective way to accurately determine IGF1 mRNA levels, in connection with the methods of the present invention, as discussed herein, is through use of quantitative real-time PCR; such methods form part of the present invention. Quantitative real time PCR can be performed in the presence of a double stranded DNA-binding dye that fluoresces upon binding to the DNA (e.g., SYBR® Green I; SYBR® Gold; or YO (Oxazole Yellow). The SYBR® Gold excitation maxima for dye-nucleic acid complexes are at about 495 nm in the visible and about 300 nm, in the ultraviolet; and the emission maximum is about 537 nm. The oxazole yellow excitation maxima for dye-nucleic acid complexes are at about 489 nm and the emission maximum is about 509 nm. Thus, for example, in real-time PCR using SYBR green I, the DNA-dye complex absorbs blue light at a wavelength of 497 nm (maximum) and emits green light at a wavelength of 520 nm (maximum). The present invention comprises embodiments wherein real time PCR is performed with such dyes. Fluorescence increases as the dye binds to the increasing amount of amplified DNA in the reaction tube. Thus, it is possible to determine the quantity of PCR amplicons present after each round of amplification. Since the cycle at which PCR enters log linear amplification is directly proportional to the amount of starting template, in an embodiment of the invention, one determines the concentration of an unknown sample by comparing it to a standard curve generated by dilutions of known amounts of product. So, for example, samples (e.g., a known control and an unknown) that differ by a factor of 2 in the original concentration of cDNA (derived from mRNA) would be 1 cycle apart; and samples that differ by a factor of 10 would be ˜3.3 cycles apart (each assuming 100% amplification efficiency) in terms of their level of DNA amplification and dye fluorescence. The cycle threshold at which an increase in fluorescence becomes exponential is called the fractional cycle number and may be designated the “Ct”. The fluorescent signal appears earlier (at lower cycle number) the higher the concentration of template. Because PCR is exponential, the correlation is logarithmic. Specifically, the logarithm of the starting template concentration is inversely proportional to the fractional cycle number which is the initial point of exponential amplification on real-time amplification curve.

An amplification curve of a real-time PCR reaction is a plot or data table that documents reaction amplification progress over time. Reaction progress can be a function of double stranded DNA binding dye (e.g., SYBR Green) fluorescence.

In an embodiment of the invention, real time PCR is performed in an apparatus that can accurately determine and monitor the level of dye fluorescence while generating amplification curves that enable the user to view run progress. Computers may be used to run the various algorithms, e.g., discussed herein, for generating values to express the concentration of IGF1 mRNA and/or the reference genes.

In an embodiment of the invention, IGF1 mRNA expression levels are determined by relative quantification real-time PCR of cDNA amplified in an RT-PCR reaction with the mRNA template. Relative quantification determines the changes in steadystate mRNA levels of a gene, e.g., across multiple samples, and expresses this amount relative to the levels of another RNA (e.g., a reference gene). In an embodiment of the invention, a negative control reaction lacking template DNA can be performed to measure background fluorescence or amplification of primer dimers and, in an embodiment of the invention, this level of fluorescence is subtracted from that of the IGF1 and/or reference gene real time PCR reactions. In general, the relative quantification is expressed as:

Ct value for IGF1 analysis/Ct value for reference gene analysis

(Bustin et al., (2005) J. Mol. Endocrinol. 34: 597-601; Orlando et al. (1998) Clin. Chem. Lab. Med. 36(5): 255-269; Vandesompele et al., Genome Biol 3(7): 0034.1-0034.11; Hellemans et al. (2007) Genome Biol. 8(2):R19; Morse et al., (2005) Anal. Biochem. 342(1): 69-77; Livak & Schmittgen (2001) Methods 25(4): 402-408). Relative quantification can be performed by the standard curve method, for example the Pfaffl method (Pfaffl at al. (2001) Nucleic Acids Res 29:e45); or the ΔΔCt method (Larionov et al. (2005) BMC Bioinformatics 6: 62; Livak & Schmittgen (2001) Methods 25: 402-408).

Another method for determining the relative level of IGF1 produced relative to that of a reference gene, which, in an embodiment of the invention, may be used in the methods discussed herein, is the second derivative maximum method (Rasmussen et al. (2001) Rapid Cycle Real-time PCR, Methods and Applications, Springer Press, Heidelberg; LightCycler Software®, Version 3.5; Roche Molecular Biochemicals (2001); Higuchi at al. (1993) Biotechnology 11:1026-1030). Using this method, the relative quantity of IGF1 (as compared to that of one or more reference genes) is expressed in terms of the fractional cycle number in which the maximal acceleration of amplification (e.g., as determined by monitoring fluorescence of a double stranded DNA binding fluorescent dye in the reaction, e.g., as discussed herein) within the log-linear phase of amplification, takes place (i.e., wherein exponential amplification can no longer be sustained). This point is determined by determining the second derivative maxima of the amplification curves. The IGF1 fractional cycle number wherein amplification acceleration is at a maximum in a given reaction over time, expressed relative to that of one or more reference genes, may be referred to as “Cq”. A similar method can be employed wherein sigmoidal and polynomial curve models are fit to the data and the second derivative maxima are then obtained (Tichopad et al. (2003) Nucl. Acids Res. 31(20): e122; Tichopad et al. (2004) Molecular and Cellular Probes 18: 45-50; Tichopad et al. (2003) Biotechn. Lett. 24: 2053-2056; Liu et al. (2002) Biochem. Biophys. Res. Commun. 294(2): 347-353; Liu et al. (2002) Anal. Biochem. 302(1): 52-59).

In an embodiment of the invention, the relative quantification of expression by real-time PCR is adjusted by the amplification efficiency of a gene (e.g., IGF1 or a reference gene). This adjustment is useful since real-time PCR quantification is based on the assumption that PCR products double each cycle. When the percentile PCR amplification efficiency is not 100%, the quantification may be adjusted to take the amplification efficiency into account. The assessment of the exact amplification efficiencies of IGF1 and reference genes can be carried out before any calculation of the normalized gene expression. Software applications that are commercially available and well known in the art, e.g., LightCycler Relative Expression Software, Q-Gene, REST and REST-XL software applications, allow the evaluation of amplification efficiency plots. For example, in a reaction with 100% efficiency, there will be a doubling of the amount of DNA at each cycle, with 90% the amount of DNA will increase from 1 to 1.9 at each cycle, and, with 80% and 70% efficiency, there will be an increase of 1.8 and 1.7 per cycle, respectively.

In a one-step RT-PCR, both steps, cDNA reverse transcriptase synthesis and amplification of the cDNA, are performed in a combined reaction with the same target specific primers and within the same reaction tube. Two-step RT-PCR, involves carrying out the reverse transcription step in one tube and the cDNA amplification step in another tube. Use of both methods is within the scope of the present invention.

Any of the methods set forth herein may include one or more of the following steps:

(a) obtaining cells of the subject's tumor, e.g., by biopsy or from an in vitro source;
(b) isolating RNA (e.g., mRNA) from said cells (e.g., by lysing the cells. RNA can be isolated by any method known in the art including precipitation; or fixing in formalin and embedding in paraffin. Embedded cells can be deparaffinization and homogenized during proteinase K incubation, then bound to a silica membrane allowing for RNA isolation, followed by washing and elution and treatment of the RNA with DNase I);
(c) generating cDNA by reverse transcribing the RNA, e.g., using oligo-dT (e.g., anchored) primers and random hexamer primers;
(d) amplifying the cDNA encoding IGF1 and, cDNA encoding one or more reference genes, e.g., selected from: CYC1, HMBS, TOP1, SDHA, GUSB, PUM1, HPRT1, ACTB, UBC, B2M, GAPDH, and TUBB2A (e.g., any of SEQ ID NOs: 14-25 or a cDNA thereof);
(e) determining the quantity of RNA encoding IGF1 and the reference gene(s) based on the level of production of the amplified cDNA; for example, in an embodiment of the invention, the progress of cDNA amplification is followed by quantitative real-time PCR; and/or
(f) normalizing the determined quantity of IGF1 with that of the reference gene(s).

In an embodiment of the invention, the cDNA is pre-amplified using PCR prior to amplifying in step (d); e.g., for about 10 cycles.

In an embodiment of the invention, the tumor, in whose cells IGF1 mRNA is determined, is osteosarcoma, rhabdomyosarcoma, neuroblastoma, kidney cancer, leukemia, renal transitional cell cancer, bladder cancer, WiInn's cancer, ovarian cancer, pancreatic cancer (e.g., where in the subject is administered the IGF1R inhibitor (e.g., MK0646) in association with gemcitabine, and optionally, ridaforolimus), breast cancer, prostate cancer, bone cancer, lung cancer, gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer.

The present invention provides a method for treating a tumor (e.g., as set forth above), in a subject (e.g., a human) in need of such treatment (e.g., whose tumor is sensitive to an IGF1R inhibitor or who is likely to achieve a positive clinical outcome upon IGF1R inhibitor therapy), that expresses IGF1 mRNA comprising administering a therapeutically effective amount of an IGF1R inhibitor (e.g., dalotuzumab) to said subject; if the fractional cycle number of a real time polymerase chain reaction amplification of IGF1 cDNA, that was reverse transcribed from IGF1 mRNA from a cell of said tumor, normalized relative to that of one or more reference genes (e.g., CYC1, HMBS, TOP1, SDHA, GUSB, PUM1, HPRT1, ACTB, UBC, B2M, GAPDH, or TUBB2A (e.g., any of SEQ ID NOs: 14-25 or a cDNA thereof)), in which acceleration of amplification is at a maximum, is at or below about 2.87 or at or below about 1.83 to about 2.03.

The present invention also provides a method for selecting a subject with a tumor for treatment with an IGF1R inhibitor (e.g., dalotuzumab) (e.g., whose tumor is sensitive to an IGF1R inhibitor or who is likely to achieve a positive clinical outcome upon IGF1R inhibitor therapy) comprising selecting the subject for treatment of the tumor with the IGF1R inhibitor if the fractional cycle number of a real time polymerase chain reaction amplification of IFG1 cDNA, that was reverse transcribed from IGF1 mRNA from a cell of said tumor, normalized relative to that of one or more reference genes (e.g., CYC1, HMBS, TOP1, SDHA, GUSB, PUM1, HPRT1, ACTB, UBC, B2M, GAPDH, or TUBB2A (e.g., any of SEQ ID NOs: 14-25 or a cDNA thereof)), in which acceleration of amplification is at a maximum, is at or below about 2.87 or at or below about 1.83 to about 2.03 (in an embodiment of the invention, if not, the subject is not selected for the IGF1R inhibitor therapy). Optionally, the method further comprises administering a therapeutically effective amount of IGF1R inhibitor to the selected subject.

The present invention provides a method for selecting a therapy for a subject with a tumor (e.g., whose tumor is sensitive to an IGF1R inhibitor or who is likely to achieve a positive clinical outcome upon IGF1R inhibitor therapy) comprising selecting an IGF1R inhibitor (e.g., dalotuzumab) for treatment of the tumor in the subject if the fractional cycle number of a real time polymerase chain reaction amplification of IGF1 cDNA, that was reverse transcribed from IGF1 mRNA from a cell of said tumor, normalized relative to that of one or more reference genes (e.g., CYC1, HMBS, TOP1, SDHA, GUSB, PUM1, HPRT1, ACTB, UBC, B2M, GAPDH, or TUBB2A (e.g., any of SEQ ID NOs: 14-25 or a cDNA thereof)), in which acceleration of amplification is at a maximum, is at or below about 2.87 or at or below about 1.83 to about 2.03 (in an embodiment of the invention, if not, the IGF1R inhibitor therapy is not selected). Optionally, the method further comprises administering a therapeutically effective amount of the selected IGF1R inhibitor to the subject.

The present invention further provides a method for evaluating the sensitivity of tumor cells to IGF1R inhibitor (e.g., dalotuzumab) therapy. The method provides, in an embodiment of the invention, determining that the tumor cells are sensitive to the IGF1R inhibitor if the fractional cycle number of a real time polymerase chain reaction amplification of IGF1 cDNA, that was reverse transcribed from IGF1 mRNA from a cell of said tumor, normalized relative to that of one or more reference genes (e.g., CYC1, HMBS, TOP1, SDHA, GUSB, PUM1, HPRT1, ACTB, UBC, B2M, GAPDH, or TUBB2A (e.g., any of SEQ ID NOs: 14-25 or a cDNA thereof)), in which acceleration of amplification is at a maximum, is at or below about 2.87 or at or below about 1.83 to about 2.03; and, if not, determining that the cells are not sensitive. As discussed above, this method may include any one or more of the following steps: (a) obtaining cells of the subject's tumor; (b) isolating RNA (e.g., mRNA) from said cells; (c) generating cDNA by reverse transcribing the RNA, e.g., using oligo-dT (e.g., anchored) primers and random hexamer primers; (d) amplifying the reverse transcribed cDNA encoding IGF1 and encoding one or more reference genes, e.g., selected from: CYC1, HMBS, TOP1, SDHA, GUSB, PUM1, HPRT1, ACTS, UBC, B2M, GAPDH, and TUBB2A (e.g., any of SEQ ID NOs: 14-25 or a cDNA thereof) in a real time polymerase chain reaction.

The present invention provides a method for predicting whether a subject with a tumor will experience a positive clinical outcome by treatment with an IGF1R inhibitor comprising determining that the subject will experience the positive clinical outcome if the fractional cycle number of a real time polymerase chain reaction amplification of IGF1 cDNA, that was reverse transcribed from IGF1 mRNA from a cell of said tumor, normalized relative to that of one or more reference genes (e.g., CYC1, HMBS, TOP1, SDHA, GUSB, PUM1, HPRT1, ACTB, UBC, B2M, GAPDH, or TUBB2A (e.g., any of SEQ ID NOs: 14-25 or a cDNA thereof)), in which acceleration of amplification is at a maximum, is at or below about 2.87 or at or below about 1.83 to about 2.03.

### In Vitro Methods

The present invention also provides in vitro assays for determining whether a given in vitro tumor cell or tissue (e.g., which has been obtained at some point from an in vivo source, such as the body of a subject) expresses IGF1 RNA at a sufficient level indicating that growth, survival or metastasis of the tumor cells would be sensitive to an IGF1R inhibitor. In an embodiment of the invention, the method comprises quantitating the expression level of IGF1 RNA in isolated tumor cells or tissue and determining, on this basis, whether the growth, survival or metastasis of the tumor would be sufficiently sensitive to an IGF1R inhibitor.

As is discussed herein, IGF1 mRNA expression in the in vitro tumor cells can be determined using real time PCR amplification of cDNA encoding the IGF1 and one or more reference genes. In an embodiment of the invention, if the fractional cycle number of a real time polymerase chain reaction amplification of IGF1 cDNA, that was reverse transcribed from IGF1 mRNA from a cell of said tumor, normalized relative to that of one or more reference genes (e.g., CYC1, HMBS, TOP1, SDHA, GUSB, PUM1, HPRT1, ACTB, UBC, B2M, GAPDH, or TUBB2A (e.g., any of SEQ ID NOs: 14-25 or a cDNA thereof)), in which acceleration of amplification (e.g., as determined by monitoring fluorescence of a double stranded DNA binding fluorescent dye in the reaction, e.g., as discussed herein) is at a maximum, is at or below about 2.87 or at or below about 1.83 to about 2.03, then the in vitro tumor cell being analyzed is determined to be sensitive to IGF1R inhibitor and, if not, the cell is determined not to be sufficiently sensitive.

For example, in an embodiment of the invention, an in vitro method of the present invention comprises:

(a) obtaining tumor cells (e.g., from a subjects tumor), e.g., by biopsy, and optionally purifying, treating or culturing the cells in vitro;
(b) isolating RNA (e.g., mRNA) from said cells (e.g., by lysing the cells. RNA can be isolated by any method known in the art including precipitation; or fixing in formalin and embedding in paraffin. Embedded cells can be deparaffinization and homogenized during proteinase K incubation, then bound to a silica membrane allowing for RNA isolation, followed by washing and elution and treatment of the RNA with DNase I);
(c) generating cDNA by reverse transcribing the RNA, e.g., using oligo-dT (e.g., anchored) primers and random hexamer primers;
(d) amplifying the cDNA encoding IGF1 and, cDNA encoding one or more reference genes, e.g., selected from: CYC1, HMBS, TOP1, SDHA, GUSB, PUM1, HPRT1, ACTB, UBC, B2M, GAPDH, and TUBB2A (e.g., any of SEQ ID NOs: 14-25 or a cDNA thereof); and
(e) determining the quantity of RNA encoding IGF1 relative to that of the reference gene(s) based on the level of production of the amplified cDNA; e.g., using real time PCR, for example, as discussed herein.

In an embodiment of the invention, the cDNA is pre-amplified using PCR prior to amplifying in step (d); e.g., for about 10 cycles.

In an embodiment of the invention, the RT-PCR amplification efficiency of IGF1 and/or reference gene RNA is estimated, e.g., using a reference RNA sample, and the efficiency calculation is used to correct the quantity of IGF1 and reference gene amplification in a real time PCR assay.

The present invention also comprises a kit for performing any of the in vitro methods set forth herein. For example, in an embodiment of the invention, the kit comprises an IGF1R inhibitor and instructions for performing the method.

### IGF1R Inhibitors

The present invention includes methods wherein an IGF1R inhibitor is used. In an embodiment of the invention, the IGF1R inhibitor is an antibody or antigen-binding fragment that binds specifically to IGF1R.

In an embodiment of the invention, the IGF1R inhibitor is dalotuzumab (MK0646; CAS no. 1005389-60-5), robatumumab, figitumumab, cixutumumab, ganitumab, AVE1642, OSI-906, NVP-AEW541 or NVP-ADW742.

In an embodiment of the invention, the IGF1R inhibitor comprises the light chain CDRs and/or the heavy chain CDRs; and/or the light chain variable region and/or the heavy chain variable region of the immunoglobulin chains in any of the antibodies selected from dalotuzumab (MK0646; CAS no. 1005389-60-5), robatumumab, figitumumab, cixutumumab and ganitumab; or from the light and/or heavy chain immunoglobulins set forth below:

| LIGHT CHAIN | |
|---|---|
| (SEQ ID NO: 1) | |
| 1 | DIVMTQSPLS LPVTPGEPAS ISCRSSQSIV |
| | HSNGNTYLQW YLQKPGQSPQ |
| 51 | LLIYKVSNRL YGVPDRFSGS GSGTDFTLKI |
| | SRVEAEDVGV YYCFQGSHVP |
| 101 | WTFGQGTKVE IKRTVAAPSV FIFPPSDEQL |
| | KSGTASVVCL LNNFYPREAK |
| 151 | VQWKVDNALQ SGNSQESVTE QDSKDSTYSL |
| | SSTLTLSKAD YEKHKVYACE |
| 201 | VTHQGLSSPV TKSFNRGEC |

In an embodiment of the inveniton, the CDRs are underscored.

| HEAVY CHAIN | |
|---|---|
| (SEQ ID NO: 2) | |
| 1 | QVQLQESGPG LVKPSETLSL TCTVSGYSIT |
| | GGYLWNWIRQ PPGKGLEWIG |
| 51 | YISYDGTNNY KPSLKDRVTI SRDTSKNQFS |
| | LKLSSVTAAD TAVYYCARYG |
| 101 | RVFFDYWGQG TLVTVSSAST KGPSVFPLAP |
| | SSKSTSGGTA ALGCLVKDYF |
| 151 | PEPVTVSWNS GALTSGVHTF PAVLQSSGLY |
| | SLSSVVTVPS SSLGTQTYIC |
| 201 | NVNHKPSNTK VDKRVEPKSC DKTHTCPPCP |
| | APELLGGPSV FLFPPRPKDT |
| 251 | LMISRTPEVT CVVVDVSHED PEVKFNWYVD |
| | GVEVHNAKTK PREEQYNSTY |
| 301 | RVVSVLTVLH QDWLNGKEYK CKVSNKALPA |
| | PIEKTISKAK GQPREPQVYT |
| 351 | LPPSREEMTK NQVSLTCLVK GFYPSDIAVE |
| | WESNGQPENN YKTTPPVLDS |
| 401 | DGSFFLYSKL TVDKSRWQQG NVFSCSVMHE |
| | ALHNHYTQKS LSLSPGK |

In an embodiment of the inveniton, the CDRs are underscored.

In an embodiment of the invention, wherein the IGF1R inhibitor is an antibody or antigen-binding fragment, the light chain immunoglobulin variable domain is linked to a light chain immunoglobulin constant domain selected from the group consisting of a kappa chain and lambda chain and/or wherein the heavy chain immunoglobulin variable domain is linked to a heavy chain immunoglobulin constant domain selected from the group consisting of a gamma-1 chain, a gamma-2 chain, a gamma-3 chain and a gamma-4 chain.

In an embodiment of the invention, the antibody or antigen-binding fragment is a monoclonal antibody, a recombinant antibody, a labeled antibody, a bivalent antibody, a polyclonal antibody, a bispecific antibody, a chimeric antibody, an anti-idiotypic antibody, a humanized antibody, a bispecific antibody, a camelized single domain antibody, a diabody, an scfv, an scfv dimer, a dsfv, a (dsfv)₂, a dsFv-dsfv′, a bispecific ds diabody, an Fv, a nanobody, an Fab, an Fab′, an F(ab′)₂, or a domain antibody; or any of the foregoing that comprises any of the CDRs and/or heavy chain variable regions and/or light chain variable regions of the antibodies discussed herein.

### Pharmaceutical Formulation

The present invention includes methods wherein an IGF1R inhibitor is administered to a subject or selected or identified. An IGF1R inhibitor or any other chemotherapeutic agent for use in any of the methods set forth herein may be formulated with a pharmaceutically acceptable carrier or excipient or the like to make a pharmaceutical composition. Pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, at al., (eds.) (1990) Pharmaceutical Dosage Forms: Tablets Dekker, New York; and Lieberman, et al., (eds.) (1990), Pharmaceutical Dosage Forms: Disperse Systems Dekker, New York.

A pharmaceutical composition containing an IGF1R inhibitor can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intratumoral, intravenous, intraperitoneal, intramuscular) and non-parenteral (e.g., oral, transdermal, intranasal, intraocular, sublingual, inhalation, rectal and topical).

A pharmaceutical composition containing an IGF1R inhibitor can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intratumoral, intravenous, intraperitoneal, intramuscular) and non-parenteral (e.g., oral, transdermal, intranasal, intraocular, sublingual, inhalation, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include acetate, histidine (e.g., with NaCl or KCl; and with polysorbate 80; and with citrate, succinate or glycine; e.g., at pH 6.0 or 6.5), phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN-80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

In an embodiment, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

An IGF1R inhibitor (e.g., dalotuzumab) may be administered to a subject in need of such administration at any therapeutically effective dosage, for example, wherein the dosage is about 1, 5, 10 or 20 mg/kg at any frequency such as once a week (e.g., on days 1, 8 and 15). Any suitable route of administration may be used, including, for example, parenteral or non-parenteral e.g., intravenous, intramuscular, subcutaneous, or intratumoral. For example, infusion may be done intravenously over a course of about 60 or 120 minutes.

When possible, the administration and dosage of any chemotheapeutic agent is done according to the schedule listed in the product information sheet of the approved agents, in the Physicians' Desk Reference, e.g., 2012 Physicians' Desk References, 66th Edition, as well as therapeutic protocols well known in the art.

### Further Chemotherapeutic Agents

The present invention provides methods comprising administering or selecting or identifying an IGF1R inhibitor (e.g., dalotuzumab) to a subject with a tumor that expresses IGF1 at a threshold level. In an embodiment of the invention, the IGF1R inhibitor is administered or selected or identified in association with a further chemotherapeutic agent or therapeutic procedure as set forth herein.

In an embodiment of the invention, the IGF1R inhibitor is in association with any androgen/estrogen ablation therapy.

In an embodiment of the invention, the IGF1R inhibitor is in association with one or more antiandrogens. Antiandrogens include steroidal varieties such as cyproterone acetate and goserelin acetate and nonsteroidal varieties such as bicalutamide, flutamide, and nilutamide.

In an embodiment of the invention, the IGF1R inhibitor is in association with one or more luteinizing hormone-releasing hormone (LHRH) agonists (e.g., goserelin acetate, leuprolide acetate or triptorelin pamoate). LHRH agonists induce a form of castration that many men opt for in lieu of orchiectomy.

In an embodiment of the invention, the IGF1R inhibitor is in association with diethylstilbestrol.

In an embodiment of the invention, the IGF1R inhibitor is in association with one or more chemotherapeutic agents that prevent the adrenal glands from making androgens. These agents include ketoconazole and aminoglutethimide.

In an embodiment of the invention, the IGF1R inhibitor is in association with one or more estrogens (e.g., synthetic estrogen such as diethylstilbestrol) that can prevent the testicles from producing testosterone.

In an embodiment of the invention, the IGF1R inhibitor is in association with androgen-depleting agents including GnRH agonists such as leuprolide and goserelin; in association with anti-androgens such as bicalutamide, flutamide, nilutimide, MDV-3100 or cyproterone acetate; or in association with both LHRH agonists and anti-androgens.

In an embodiment of the invention, the IGF1R inhibitor is in association with anti-androgens such as bicalutamide, flutamide, nilutimide, MDV-3100, cyproterone acetate; in association with both LHRH agonists and anti-androgens; in association with the CYP17 lyase inhibitors such as abiraterone acetate, galeterone, and orteronel; or in association with ketoconazole.

In an embodiment of the invention, the IGF1R inhibitor is in association with one or more additional IGF1R inhibitors (e.g., any set forth herein).

In an embodiment of the invention, the IGF1R inhibitor is in association with docetaxel, mitoxantrone and/or prednisone.

In an embodiment of the invention, the IGF1R inhibitor is in association with an AKT inhibitor and/or a PI3 kinase (including alpha, beta, gamma and/or delta) inhibitor. AKT inhibitors include perifosine, SR13668, A-443654, triciribine phosphate monohydrate,

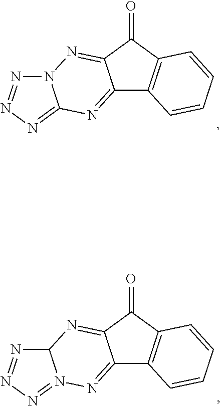

GSK690693, deguelin. PI3 kinase inhibitors include SF1126, TGX-221, PIK-75, PI-103, SN36093, IC87114, AS-252424, AS-605240, NVP-BEZ235, GDC-0941, ZSTK474, PX-866,

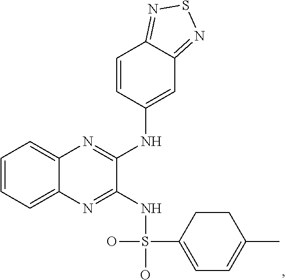 [SMILES: CC1=CC=C(S(O)(O)Nc2nc3ccccc3nc2Nc2ccc3nsnc3c2)CC1]

LY294002 and wortmannin.

In an embodiment of the invention, the IGF1R inhibitor is in association with any antiestrogen and/or selective estrogen receptor modulator (SERM), including estrogen receptor alpha antagonists and estrogen receptor beta agonists such as diarylpropionitrile, raloxifene, droloxifene (3-hydroxytamoxifen), 4-hydroxytamoxifen, pipendoxifene, ERA-923, arzoxifene, fulvestrant, acolbifene, lasofoxifene (CP-336156), idoxifene, tamoxifen or toremifene citrate.

In an embodiment of the invention, the IGF1R inhibitor is in association with erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763 or AT-9263.

In an embodiment of the invention, the IGF1R inhibitor is in association with a Notch inhibitor such as cis-3-[4-[(4-chlorophenyl)sulfonyl]-4-(2,5-difluorophenyl)cyclohexyl]propanoic acid

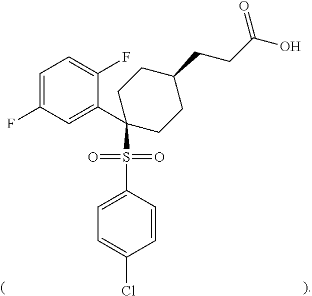 [SMILES: O=C(O)CC[C@H]1CC[C@@](c2cc(F)ccc2F)(S(=O)(=O)c2ccc(Cl)cc2)CC1]

Abraxane is an injectable suspension of paclitaxel protein-bound particles comprising an albumin-bound form of paclitaxel with a mean particle size of approximately 130 nanometers. Abraxane is supplied as a white to yellow, sterile, lyophilized powder for reconstitution with 20 mL of 0.9% Sodium Chloride Injection, USP prior to intravenous infusion. Each single-use vial contains 100 mg of paclitaxel and approximately 900 mg of human albumin. Each milliliter of reconstituted suspension contains 5 mg paclitaxel. Abraxane is free of solvents and is free of cremophor (polyoxyethylated castor oil).

In an embodiment of the invention, the IGF1 R inhibitor is in association with romidepsin, ADS-100380,

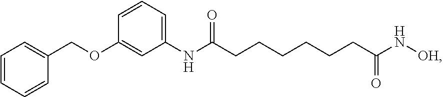 [SMILES: O=C(CCCCCCC(=O)Nc1cccc(OCc2ccccc2)c1)NO]

### CG-781, CG-1521,

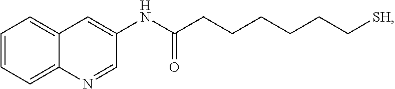 [SMILES: O=C(CCCCCCS)Nc1cnc2ccccc2c1]

scriptaid, chlamydocin, JNJ-16241199,

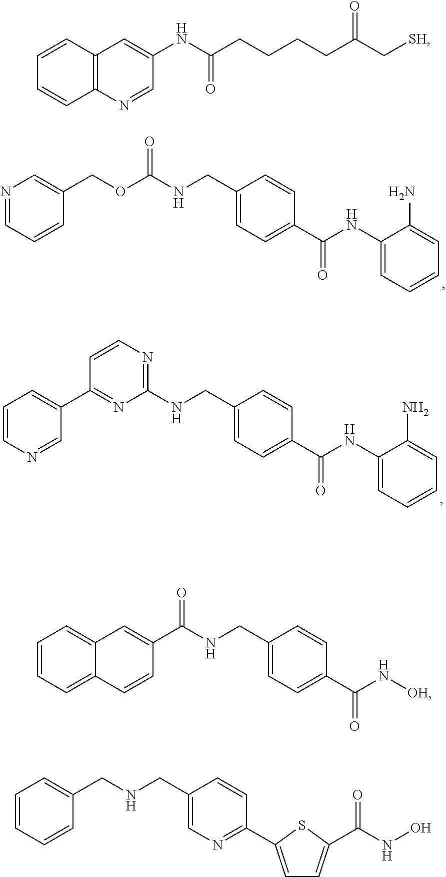

or vorinostat.

In an embodiment of the invention, the IGF1R inhibitor is in association with etoposide.

In an embodiment of the invention, the IGF1R inhibitor is in association with gemcitabine or a combination of gemcitabine in association with erlotinib. In an embodiment of the invention, the tumor is a pancreatic cancer tumor and the IGF1R inhibitor (e.g., MK0646) is in association with gemcitabine, and optionally, ridaforolimus.

In an embodiment of the invention, the IGF1R inhibitor is in association with doxorubicin; including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, the IGF1R inhibitor is in association with 5′-deoxy-5-fluorouridine.

In an embodiment of the invention, the IGF1R inhibitor is in association with vincristine.

In an embodiment of the invention, the IGF1R inhibitor is in association with temozolomide, any CDK inhibitor such as ZK-304709, Seliciclib (R-roscovitine); any MEK inhibitor such as PD0325901, AZD-6244; capecitabine; or pemetrexed.

In an embodiment of the invention, the IGF1R inhibitor is in association with camptothecin, irinotecan; a combination of irinotecan, 5-fluorouracil and leucovorin; or PEG-labeled irinotecan.

In an embodiment of the invention, the IGF1R inhibitor is in association with the FOLFOX regimen (oxaliplatin, together with infusional fluorouracil and folinic acid).

In an embodiment of the invention, the IGF1R inhibitor is in association with an aromatase inhibitor such as anastrazole, exemestane or letrozole.

In an embodiment of the invention, the IGF1R inhibitor is in association with an estrogen such as DES(diethylstilbestrol), estradiol or conjugated estrogens.

In an embodiment of the invention, the IGF1R inhibitor is in association with an anti-angiogenesis agent such as bevacizumab, the anti-VEGFR-2 antibody IMC-1C11, other VEGFR inhibitors such as: dovitinib,

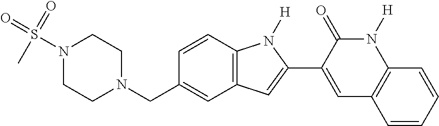 [SMILES: CS(=O)(=O)N1CCN(Cc2ccc3[nH]c(-c4cc5ccccc5[nH]c4=O)cc3c2)CC1]

3-[5-(methylsulfonylpiperadinemethyl)-indolyl]-quinolone; vatalanib, AG-013736; and the VEGF trap (AVE-0005), a soluble decoy receptor comprising portions of VEGF receptors 1 and 2.

In an embodiment of the invention, the IGF1R inhibitor is in association with a LHRH (Lutenizing hormone-releasing hormone) agonist such as goserelin acetate; leuprolide acetate; triptorelin pamoate.

In an embodiment of the invention, the IGF1R inhibitor is in association with sunitinib or sunitinib malate.

In an embodiment of the invention, the IGF1R inhibitor is in association with a progestational agent such as medroxyprogesterone acetate, hydroxyprogesterone caproate, megestrol acetate or progestins.

In an embodiment of the invention, the IGF1R inhibitor is in association with any antiestrogen and/or selective estrogen receptor modulator (SERM), including estrogen receptor alpha antagonists and estrogen receptor beta agonists such as diaryipropionitrile, raloxifene, droloxifene (3-hydroxytamoxifen), 4-hydroxytamoxifen, pipendoxifene, ERA-923, arzoxifene, fulvestrant, acolbifene, lasofoxifene (CP-336156), idoxifene, tamoxifen or toremifene citrate.

In an embodiment of the invention, the IGF1R inhibitor is in association with an anti-androgen including, but not limited to bicalutamide; flutamide; nilutamide and megestrol acetate.

In an embodiment of the invention, the IGF1R inhibitor is in association with one or more inhibitors which antagonize the action of the EGF Receptor or HER2 such as CP-724714; HKI-272; erlotinib, lapatanib, canertinib, panitumumab, erbitux, EKB-569, PKI-166, GW-572016, any anti-EGFR antibody or any anti-HER2 antibody.

In an embodiment of the invention, the IGF1R inhibitor is in association with lonafarnib or any other FPT inhibitor such as:

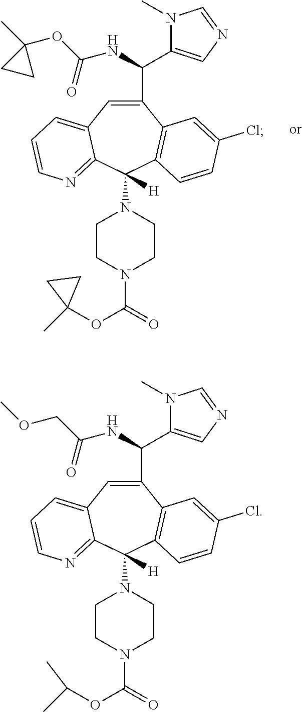 [SMILES: Cn1cncc1[C@H](NC(=O)OC1(C)CC1)C1=Cc2cccnc2[C@@H](N2CCN(C(=O)OC3(C)CC3)CC2)c2ccc(Cl)cc21 ; "or" ; COCC(=O)N[C@H](C1=Cc2cccnc2[C@@H](N2CCN(C(=O)OC(C)C)CC2)c2ccc(Cl)cc21)c1cncn1C]

Other FPT inhibitors include BMS-214662, tipifarnib.

In an embodiment of the invention, the IGF1R inhibitor is in association with Amifostine; NVP-LAQ824, suberoyl analide hydroxamic acid, valproic acid, trichostatin A, depsipeptide, sunitinib; sorafenib, KRN951, aminoglutethimide; Amsacrine; Anagrelide; Anastrozole; Asparaginase; Bacillus Calmette-Guerin (BCG) vaccine; bleomycin; Buserelin; Busulfan; Carboplatin; Carmustine; Chlorambucil; Cisplatin; cladribine; clodronate; cyclophosphamide; cyproterone; cytarabine; dacarbazine; dactinomycin; daunorubicin; diethylstilbestrol; epirubicin; fludarabine; fludrocortisone; fluoxymesterone; flutamide; hydroxyurea; idarubicin; ifosfamide; imatinib; leucovorin; leuprolide; levamisole; lomustine; mechlorethamine; melphalan; mercaptopurine; mesna; methotrexate; mitomycin; mitotane; mitoxantrone; nilutamide; octreotide; edotreotide (yttrium-90 labeled or unlabeled); oxaliplatin; pamidronate; pentostatin; plicamycin; porfimer; procarbazine; raltitrexed; rituximab; streptozocin; teniposide; testosterone; thalidomide; thioguanine; thiotepa; tretinoin; vindesine or 13-cis-retinoic acid.

In an embodiment of the invention, the IGF1R inhibitor is in association with one or more of any of: phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, semaxanib, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), any mTOR inhibitor, ridaforolimus, sirolimus, 40-O-(2-hydroxyethyl)-rapamycin, temsirolimus, AP-23573, RAD001, ABT-578; BC-210, LY294002, LY292223, LY292696, LY293684, LY293646, wortmannin, sorafenib, ZM336372, L-779,450, any Raf inhibitor; flavopiridol or 7-hydroxy staurosporine.

In an embodiment of the invention, the IGF1R inhibitor is in association with interferon (e.g., PEG-interferon).

In an embodiment of the invention, the IGF1R inhibitor is in association with one or more antiemetics including, but not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Other side effects of cancer treatment include red and white blood cell deficiency. Accordingly, in an embodiment of the invention, the IGF1R inhibitor is in association with an agent which treats or prevents such a deficiency, such as, e.g., filgrastim, PEG-filgrastim, erythropoietin, epoetin alfa or darbepoetin alfa.

In an embodiment of the invention, the IGF1R inhibitor is administered in association with anti-cancer radiation therapy. For example, in an embodiment of the invention, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In an embodiment of the invention, the radiation therapy is proton beam therapy: a type of conformal therapy that bombards the diseased tissue with protons instead of X-rays. In an embodiment of the invention, the radiation therapy is conformal external beam radiation therapy: a procedure that uses advanced technology to tailor the radiation therapy to an individual's body structures.

In an embodiment of the invention, the radiation therapy is brachytherapy: the temporary placement of radioactive materials within the body, usually employed to give an extra dose—or boost—of radiation to an area.

In an embodiment of the invention, a surgical procedure administered in association with an IGF1R inhibitor is surgical tumorectomy.

The term “in association with” indicates that the components administered in a method of the present invention (e.g., anti-IGF1R antibody or antigen-binding fragment thereof along with ridaforolimus) can be formulated into a single composition for simultaneous delivery or formulated separately into two or more compositions (e.g., a kit). Each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., wherein an anti-IGF1R antibody is administered parenterally and gosrelin acetate is administered orally).

### EXAMPLES

The present invention is intended to exemplify the present invention and not to be a limitation thereof. The methods (e.g., methods for using the IGF1 biomarker) and compositions (e.g., polypeptides, polynucleotides, plasmids, yeast cells) disclosed below fall within the scope of the present invention.

### Example 1

### Pre-Clinical: IGF1 is Associated with IGF1R Antibody, MK-0646, Sensitivity

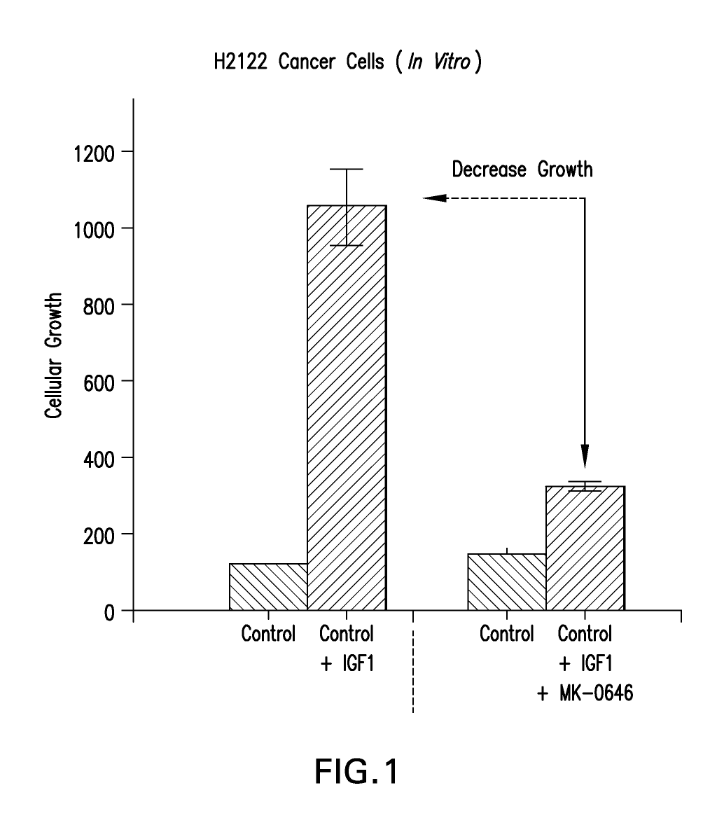

H2122 cancer cells were grown in vitro with and without IGF1 present in the growth media containing low levels of growth factors. Under these conditions, IGF1 significantly stimulated the growth of H2122 cancer cells as compared to the control (H2122 cells grown without IGF1 present in the growth media). Anti-IGF1R antibody, dalotuzumab (MK-0646), significantly blocked the IGF1-dependent proliferation of H2122 cancer cells. In contrast, MK-0646 did not significantly alter the proliferation of H2122 cells grown in the absence of IGF1. MK-0646, was capable of blocking IGF1 ability to bind directly to the IGF1 receptor. This prevented IGF1 from activating IGF1 receptor required for increasing cancer cell growth. These preclinical data provided in vitro evidence that the anti-IGF1R antibody, MK-0646 growth inhibitory effects are strongly dependent on presence of IGF1, which is required for IGF1 receptor activation (see FIG. 1).

In Vitro Methodology:

H2122 cells were obtained from the American Type Culture Collection (ATCC) and propagated according to the conditions provided by ATCC in media at 37° C. H2122 cells in low serum (2% fetal calf serum (Hyclone), at 2000 cells/well, were plated in a 96 well plate and incubated with 10 ug/ml MK-0646 or vehicle control for 96 hours in the presence or absence of IGF1 (10 ng; SIGMA). Cells were harvested at day 0 and day 4 and cell growth was measured by Cell Titer-Glo® (Invitrogen) according to manufacturer instructions. The relative cell proliferation was calculated by normalizing to day 0 levels.

In Vivo Methodology:

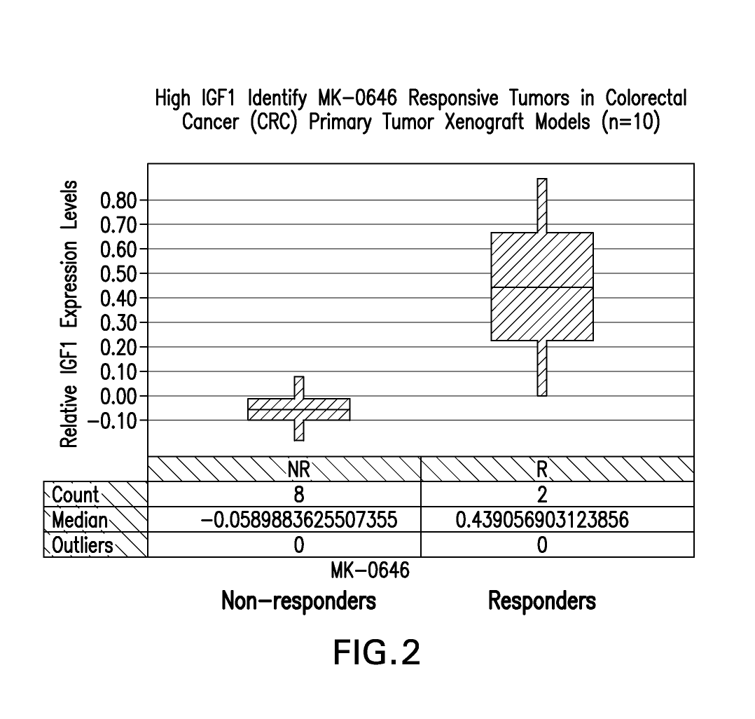

Primary colorectal xenograft tumor models were established from 10 independent colorectal tumors obtained directly from cancer patients. The relative levels of IGF1RNA expression were determined by gene expression profiling using genomic microarrays. Of the ten primary colorectal xenograft models evaluated, 2 models showed markedly increased IGF1RNA expression levels (see FIG. 2). These 2 colorectal xenograft models, that were identified to have increase IGF1RNA expression levels, among the 10 xenograft models evaluated, showed the greatest tumor growth inhibition following treatment with the anti-IGF1R antibody, MK-0646. These two tumor models demonstrated at least 50% or more tumor growth inhibition. For the 8 xenografts tumor models with low IGF1 expression, all showed minimal or no tumor growth inhibition following treatment with the anti-IGF1R antibody, MK-0646. These preclinical data provided in vivo evidence that increase sensitivity to the anti-IGF1R antibody, MK-0646, was strongly associated with increase IGF1RNA expression in the primary tumors.

Clinical and Assay Information:

Here we provide a summary of the work used to perform the IGF1 quantitative real-time PCR (rtPCR) gene expression analysis on actual clinical colorectal formalin-fixed paraffin embedded (FFPE) tissue material and provide clinical evidence supporting the idea that increased IGF1RNA expression levels, as detected directly within the tumor microenvironment, may be important for determining clinical benefit to an anti-IGF1R antibody (MK-0646)-based therapy. The document also contains a detail description of the methods used to process the samples, generate IGF1 RNA expression data and perform the IGF1 analysis.

Methods:

Below is a detail description of the forward and reverse primer sequences designed specifically to evaluate IGF1 by rtPCR using RNA isolated from FFPE tissue material.

TABLE 1
Forward and reverse primer sequences
for determining IGF1 by RT-PCR.

| | | | | | | Self |
|---|---|---|---|---|---|---|
| | | | | | Self | 3′ |
| | Sequence | | | | comple- | comple- |
| | (5′->3′) | Length | Tm | GC% | mentarity | mentarity |
| Primer pair 1 | | | | | | |
| Forward primer | TGTGGAGACAGG | 24 | 55.05 | 45.83 | 3.00 | 0.00 |
| | GGCTTTTATTTC | | | | | |
| | (SEQ ID NO: 3) | | | | | |
| Reverse primer | GCAGCACTCATC | 20 | 56.38 | 60.00 | 5.00 | 4.00 |
| | CACGATGC | | | | | |
| | (SEQ ID NO: 8) | | | | | |

>NM 000618.3 Homo sapiens insulin-like growth factor 1
(somatomedin C) (IGF1), transcript variant 4, mRNA
product length = 93
Forward primer 1 TGTGGAGACAGGGGCTTTTATTTC 24 (SEQ ID NO: 4)
Template 415 ... 438
Reverse primer 1 GCAGCACTCATCCACGATGC 20 (SEQ ID NO: 9)
Template 507 ... 488
>NM 001111285.1 Homo sapiens insulin-like growth factor 1
(somatomedin C) (IGF1), transcript variant 3, mRNA
product length = 93
Forward primer 1 TGTGGAGACAGGGGCTTTTATTTC 24 (SEQ ID NO: 5)
Template 415 ... 438
Reverse primer 1 GCAGCACTCATCCACGATGC 20 (SEQ ID NO: 10)
Template 507 ... 488
>NM 001111284.1 Homo sapiens insulin-like growth factor 1
(somatomedin C) (IGF1), transcript variant 2, mRNA
product length = 93
Forward primer 1 TGTGGAGACAGGGGCTTTTATTTC 24 (SEQ ID NO: 6)
Template 298 ... 321
Reverse primer 1 GCAGCACTCATCCACGATGC 20 (SEQ ID NO: 11)
Template 390 ... 371
>NM 001111283.1 Homo sapiens insulin-like growth factor 1
(somatomedin C) (IGF1), transcript variant 1, mRNA
product length = 93
Forward primer 1 TGTGGAGACAGGGGCTTTTATTTC 24 (SEQ ID NO: 7)
Template 415 ... 438
Reverse primer 1 GCAGCACTCATCCACGATGC 20 (SEQ ID NO: 12)
Template 507 ... 488

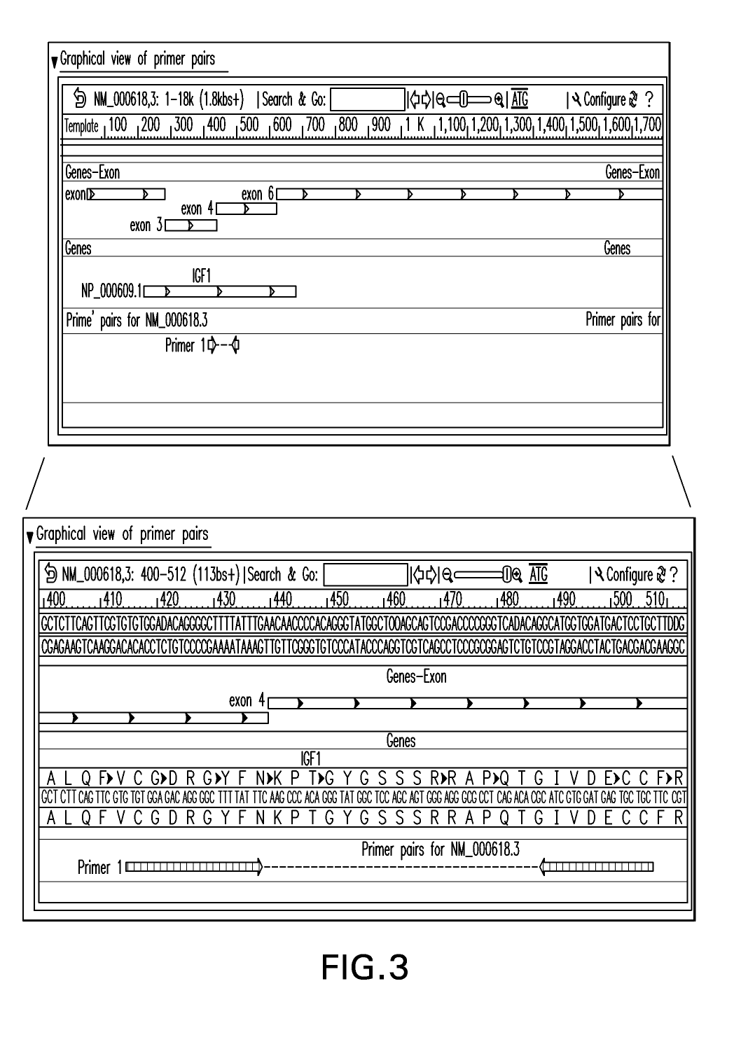

FIG. 3 sets forth the results from an in silica BLAST analysis with the IGF1 forward and reverse primer sequences described in this document. The graph shows the location where the IGF1 forward and reverse primers bind directly on the IGF1 gene sequence and provides a graphical view of the expected IGF1 amplification product (predicted size to be between 92-93 base pairs in length).

FFPE Tissue RNA Isolation:

Pieces of human tumor disease tissue were obtained from 12 individual patients diagnosed with colorectal cancer. The tissue obtained was then submerged in 10% neutral-buffered formalin for a maximum of 24 hours and embedded in IHC-grade paraffin (known as a FFPE tissue sample). Sections of the FFPE tissue sample were then cut from a block—ranging in thickness from 5 to 10 microns, and placed on positively- or negatively-charged glass slides. For each FFPE tissue section on a glass slide—marcodisection and deparaffinization was performed using xylene. The tissue was disrupted and homogenized during proteinase K incubation. A chaotrophic salt was used to bind nucleic acids to a silica membrane allowing for RNA isolation. Following washing and elution, the RNA was treated with DNase I to remove any residual genomic contamination. The RNA was eluted using a low salt elution buffer.

RNA Quality Controls:

All total RNA samples were analyzed for concentration and purity by assessing the A₂₆₀/A₂₈₀ on the Nanodrop™ 1000 (Thermo Scientific®). All samples were deemed to be of sufficient quality to proceed for further processing.

Reverse Transcription:

96 ng of total RNA isolated from 12 Colorectal FFPE samples was reverse transcribed using the Transcriptor First Strand cDNA synthesis kit (Roche Applied Science). A combination of anchored oligo-dT and random hexamer priming was used for reverse transcription to ensure optimal priming of fragmented and modified nucleic acids found in FFPE material.

Pre-Amplification:

The following twelve reference genes: CYC1, HMBS, TOP1, SDHA, GUSB, PUM1, HPRT1, ACTB, UBC, B2M, GAPDH, TUBB2A and the target gene of interest IGF1, in the reverse transcribed cDNA from the 12 colorectal FFPE samples, were pre-amplified for 10 cycles. Following pre-amplification, each sample was diluted 1:5 using TE buffer prior to use for rtPCR analysis.

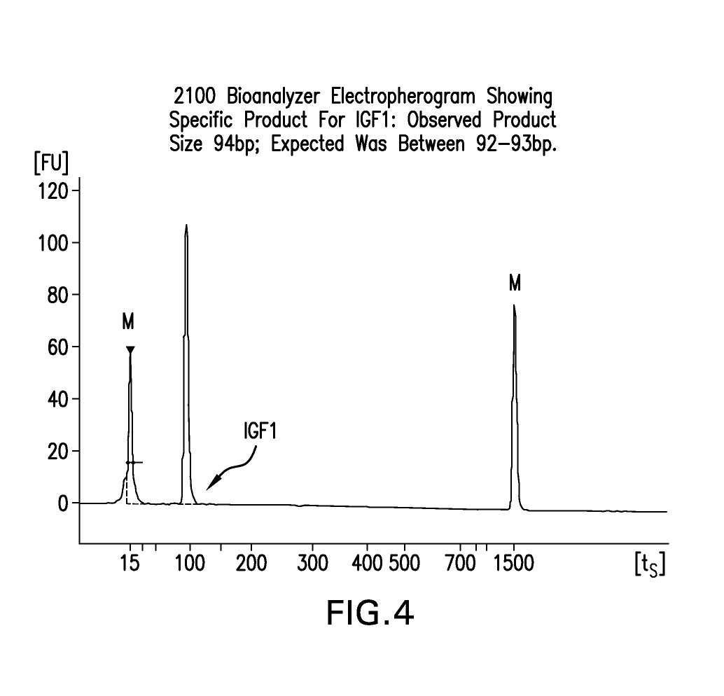

The IGF1 rtPCR assay was designed to avoid any appreciable amplification of pseudogenes or other targets. Amplified product from different PCR products was assessed to ensure that the IGF1 single product of the appropriate size was specifically amplified. The IGF1 rtPCR assay was validated empirically using the 2100 bioanalyzer, DNA 1000 chip and identified the actual single amplified product using the IGF1 primers described in this document to be 94 base pairs in length—confirms IGF1 (Agilent; see FIG. 4).

Amplification Efficiency of IGF1 Target Gene:

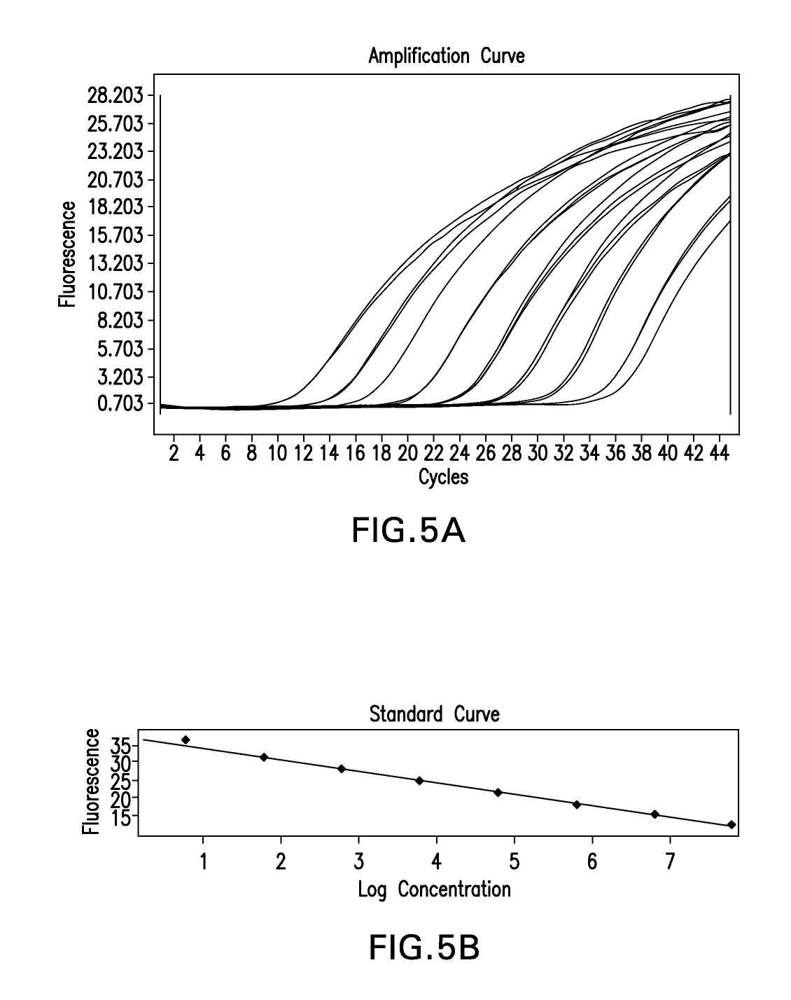

The amplification efficiencies of IGF1 were previously estimated, experimentally, using the slope from standard curves generated using Universal Human Reference RNA (Stratagene). FIG. 5 showed the efficiencies for IGF1 rtPCR assay.

rtPCR Processing:

The levels of twelve reference gene transcripts genes (CYC1, HMBS, TOP1, SDHA, GUSB, PUM1, HPRT1, ACTB, UBC, B2M, GAPDH, TUBB2A) in addition to the target transcript, IGF1, were determined by real time PCR for each of the pre-amplified FFPE colorectal samples. In addition to each sample, pre-amplified reverse transcription negative samples (RTneg) were included with each reference and the IGF1 target gene. A no template control (NTC) was run as a negative control for each target and a Universal Human Reference RNA (UHR) triplicate was run as a positive control for each target. All samples were processed using an ‘All Samples’ design where all samples were amplified on a single plate to avoid the need for inter-plate calibrators (IPCs). All the rtPCR master mixes were assembled and dispensed using the CAS-4200 liquid handling system in a ‘clean’ room free from amplified product. Each real time PCR reaction was performed in triplicate. The pre-amplified cDNA was added by a CAS-1200 liquid handling system in a separate room to minimize the risk of reagent contamination. The real time PCR thermal cycling was performed using a Roche Lightcycler® 480 I running software version 1.5. The data obtained were analyzed using the absolute quantification second derivative maximum method to obtain Cq values.

Normalized expression data was obtained on all 12 of the colorectal FFPE samples tested. The values presented in Table 2 below represent the IGP1 expression level relative to the reference genes and range from −0.575 (highest IGF1 expression) to 9.033 (lowest IGF1 expression).

TABLE 2
Normalized IGF1 levels (Cq) for each of 12 patients.

| Number of | | |
|---|---|---|
| Colorectal | | |
| Cancer | Subject (patient) | IGF1 Cq |
| Samples | ID | values |
| 1 | 14279 | −0.575 |
| 2 | 14184 | −0.385 |
| 3 | 13090 | 0.237 |
| 4 | 14265 | 0.305 |
| 5 | 13013 | 0.968 |
| 6 | 13073 | 0.985 |
| 7 | 14183 | 5.948 |
| 8 | 14323 | 6.407 |
| 9 | 15320 | 6.906 |
| 10 | 15316 | 7.182 |
| 11 | 16400 | 8.4 |
| 12 | 14303 | 9.033 |

The 12 human colorectal subjects with IGF1 normalized real time PCR data had participated in a clinical study evaluating how well the anti-IGF1R antibody, MK-0646, in combination with standard of care agents (cetuximab and irinotecan) worked in treating patients with metastatic colorectal cancer. All 12 subjects received and were treated with the anti-IGF1R antibody MK-0646-based therapy and clinical outcome data were available. The patients above were classified into two groups. One group of patients (#1-6 in Table above) was classified to have “increase” IGF1 expression levels (Cq value ranged from −0.575 to 0.985). The second groups of patients (#7-12 in Table above) were classified as having “low” IGF1 expression levels (Cq value range from 5.95 to 9.03). The IGF1 Cq values were reflective of the relative IGF1RNA levels detected within the colorectal tumor sampled directly from the patient (tumor microenvironment). The diseased tissue was obtained from the primary colorectal tumor prior to receiving the anti-IGF1R antibody, MK-0646-based therapy.

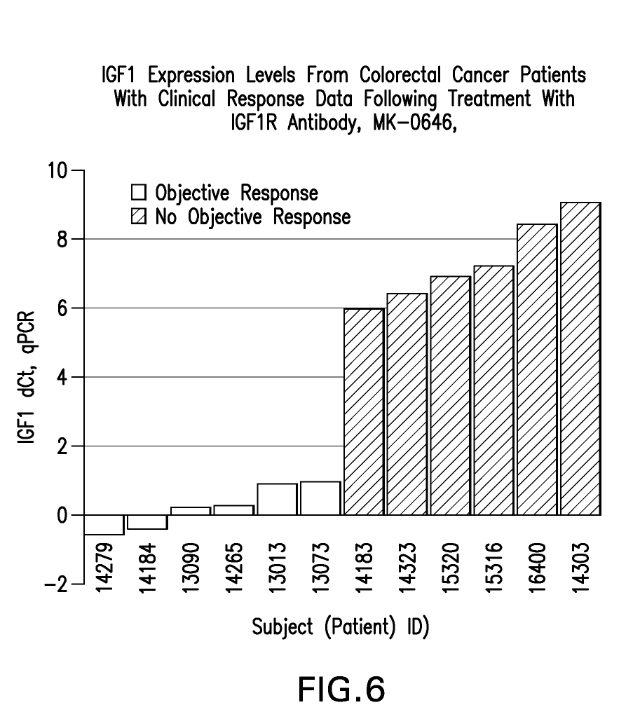

FIG. 6 shows the IGF1 Cq values from the 12 colorectal cancer patient FFPE samples evaluated with clinical response (objective response=tumor shrinkage; no objective response=no tumor shrinkage) following treatment with anti-IGF1R antibody, MK-0646-based therapy.

For the patients classified as having increase IGF1RNA expression levels (Cq values range from −0.575 to 0.985 in the bar graph), clinical benefit was observed by confirmed partial responses & tumor size shrinkage (PR=Objective response) lasting at least 6 weeks (criteria Response Evaluation Criteria In Solid Tumors (RECIST) v1.0) during treatment with the anti-IGF1R antibody, MK-0646-based therapy. For patients classified as having low IGF1RNA expression levels (Cq values range from 5.95 to 9.03), no tumor shrinkage or clinical activity was observed, all the patients with low IGF1 expression experienced progressive disease (criteria RECIST v1.0) during treatment with the anti-IGF1R antibody, MK-0646-based therapy.

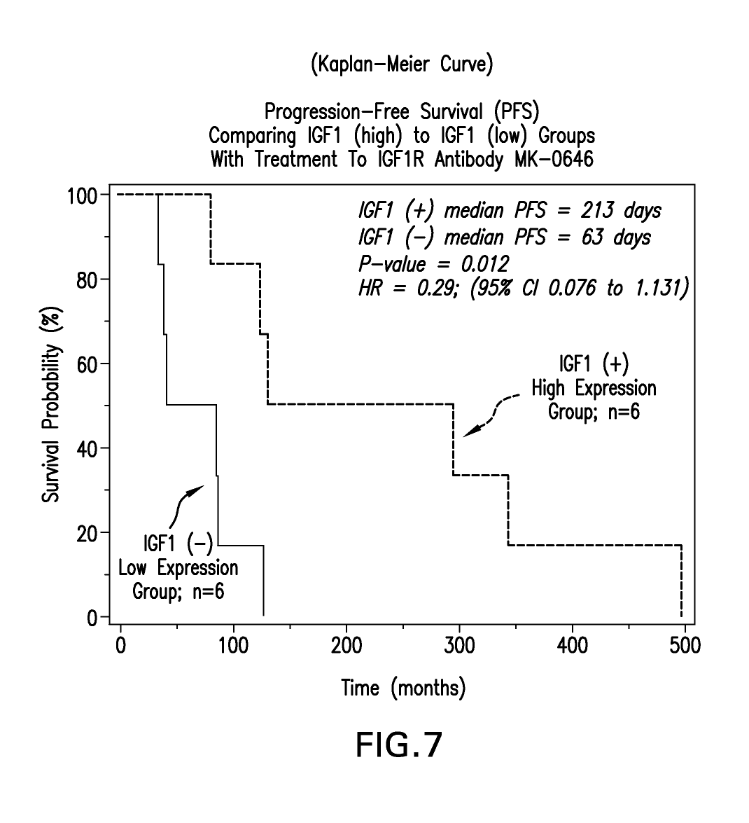

Progression-free survival (PFS) was evaluated with treatment with the anti-IGF1R antibody, MK-0646-based therapy for the 12 colorectal cancer patients studied. This was done by comparing PFS for patient groups defined to have either increase (high=+) IGF1 expression (n=6) or low IGF1 expression (n=6). An improved progression-free survival time (median PFS was 213 days) was observed for the patients with increase IGF1 expression compared to the patients with low IGF1 expression (median PFS was only 63 days). The PFS hazard ratio (HR) was 0.29 with a P-value=0.012; 95% Cl 0.07 to 1.1. See FIG. 7.

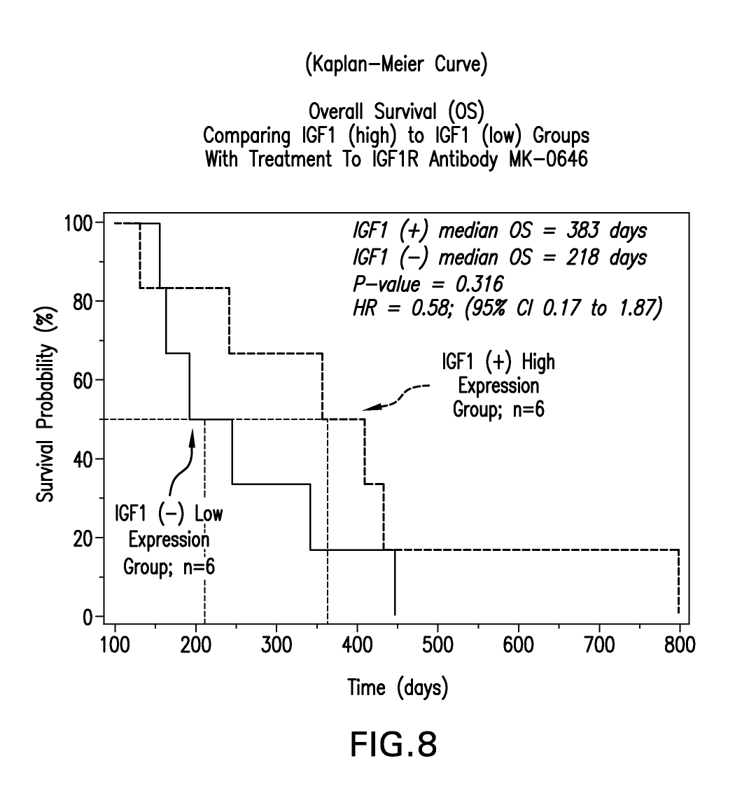

The association between increase IGF1 expression levels with improved overall survival was also evaluated on the same 12 colorectal cancer patients. Observed for the group of patients defined to have increase IGF1 expression (n=6) a trend toward improved overall survival (median OS=383 days) vs. the group of patients defined to have low IGF1 expression levels (median OS=218 days). The overall survival hazard ratio (HR) was 0.58, the P-value did not reach significance (P=0.316; 95% Cl 0.017 to 1.87). See FIG. 8.

In addition for the 6 patients with highest IGF1RNA expression who experienced clinical benefit to the anti-IGF1R antibody, MK-0646, 4 out 6 (67%) had been diagnosed with having rectal (rectum) cancer compared to 0 out 6 (0%) for the patients with low levels of IGF1 expression. These data may suggest that increased IGF1 levels are higher and more frequent in tumors located in the rectum compared to colon.

TABLE 3
Normalized IGF1 levels (Cq) for each of 12 patients
with tumor locations indicated.

| | | IGF1 Cq |
|---|---|---|
| Subject ID | Tumor Location | values |
| 14279 | Colon | −0.575 |
| 14184 | Rectum | −0.385 |
| 13090 | Rectum | 0.237 |
| 14265 | Colon | 0.305 |
| 13013 | Rectum | 0.968 |
| 13073 | Rectum | 0.985 |
| 14183 | Colon | 5.948 |
| 14323 | Colon | 6.407 |
| 15320 | Colon | 6.906 |
| 15316 | Colon | 7.182 |
| 16400 | Colon | 8.4 |
| 14303 | Colon | 9.033 |

Conclusion:

This document provides both pre-clinical and clinical evidence supporting the idea that increased levels of IGF1 are useful for predicting sensitivity to agents targeting the IGF1 receptor pathway, such as the anti-IGF1R antibody MK-0646. A sensitive and highly specific IGF1 rtPCR assay method that is capable of quantization the amount of IGF1 RNA transcript present directly within the tumor microenvironment using clinical FFPE tissue specimens was also provided. The rtPCR data suggested that increased IGF1 expression occurred more frequently in tumors located from the rectum. Our idea is that an accurate & sensitive measurement to determine the levels of IGF1 can be acquired by use of the methods outlined in this document and provide a means by which one could detect whether or not IGF1 is present within the tumor microenvironment. Accurately determining the levels of IGF1 expression in a clinical tumor specimens will be a critical requirement in helping to identify cancer patients who are most likely to benefit from cancer agents targeting the IGF1 pathway, such as the anti-IGF1R antibody MK-0646.

### Example 2

### Tumor IGF-1 Expression as a Predictive Biomarker for IGF1R-Directed Therapy in Advanced Pancreatic Cancer (APC)

Background:

IGF1 up-regulates PC proliferation and invasiveness through activation of PI3K/Akt signaling pathway and down-regulates PTEN. We investigated IGF1 expression in tissue and blood as potential predictive markers in a phase II study of IGF1R-directed monoclonal antibody, MK-0646 in APC. Prior phase I studies established the MTD of MK0646 at 5 mg/kg with Gemcitabine (G) and Erlotinib (E) and 10 mg/kg with G alone.

Methods:

Patients (pts) with stage 1V, previously untreated APC, ECOG PS 0-1, adequate hematologic and organ function were enrolled.

Arm A: G 1000 mg/m² over 100 min, weekly×3, MK-0646 weekly×4;

Arm B: G 1000 mg/m² and MK-0646+E 100 mg daily;

Arm C (control) was G 1000 mg/m²+E 100 mg.

Cycles were repeated every 4 weeks. Patients were equally randomized in the 3 arms. The primary study objective was progression-free survival (PFS). Pre-treatment peripheral blood samples measured for IGF1 level by ELISA in all cases; archival core biopsies were analyzed for IGF1 mRNA expression. RNA extraction from FFPE samples used the Roche Transcriptor First Strand cDNA Synthesis Kit. TaqMan PreAmp technique was used to amplify target cDNA prior to TaqMan RT-PCR analysis. Cox proportional hazards model for PFS analyzed the interaction between tissue IGF1 expression and treatment.

Results:

50 patients were enrolled (A=17, B=17, C=16 pts). Median PFS of arms A, B and C were 5.5 months (95% Cl: 3.9—NA), 3.0 months (95% Cl 1.8-5.6) and 2.0 months (95% Cl: 1.8—NA), respectively (log-rank test; p-value=0.17). Median OS of A was 11.3 months (95% Cl: 8.9—NA), B 8.9 months (95% Cl: 5.3—NA) and C 5.7 months (95% Cl: 2.0—NA) (log-rank test; p-value=0.44). 35 archival core biopsies were analyzed, 21 had adequate tissue for analysis. Using a Multivariable Cox proportional hazards model for PFS, where IGF1 was dichotomized at the median, there was a 76% reduction in the risk of disease of progression or death in arm A as compared with the control (arm C) with a p=0.16. When IGF1 was fitted as a continuous variable, this reduction was 96% (p=0.08).

Conclusion:

Tissue expression of IGF1 level represents a promising predictive biomarker for IGF1R inhibitor therapy in APC.

### Example 3

### Low RAS and High IGF as Biomarkers for Dalotuzumab (MK-0646) and Ridaforolimus (MK-8669) Combination Therapy in Ovarian Carcinoma

Translational work has suggested that low RAS activity, as determined by a RAS gene expression signature score, and high IGF levels may enrich for response to combination therapy with the mTOR inhibitor ridaforolimus and the IGF1R monoclonal antibody dalotuzumab (Loboda et al., Clin. Pharmacol. Ther. 2009; 86 (1):92-6; Ebbinghaus et al., Mol. Cancer Ther. 10 (11), Suppl 1, 1158). Consistent with these observations, clinical responses have been noted for several ER⁺ breast and ovarian cancer patients, indications that may be enriched for low RAS and high IGF, in a Phase I trial for ridaforolimus and dalotuzumab combination therapy (Ebbinghaus et al., Mol. Cancer Ther. 10 (11), Suppl 1, 1158).

TABLE 4
RAS Gene Expression Score, IGF Levels, and Response to
Ridaforolimus and Dalotuzumab Combination Therapy in
Patient-Derived Xenograft (PDX) Models of Ovarian Carcinoma.

| | | | | | Combination |
|---|---|---|---|---|---|
| | | RAS | | | Response |
| PDX Ovarian | RAS/RAF | pathway | | | (% tumor growth |
| Tumor Model | mutation | score | IGF1 | IGF2 | inhibition, TGI) |
| ST025 | wt | −0.39 | 1.1 | 3.4 | 90 |
| ST015 | wt | −0.38 | 1.6 | 3.1 | 139 |
| ST187 | wt | −0.34 | 2.6 | 1.6 | 74 |
| ST038 | wt | −0.24 | 2.7 | 2.1 | 82 |
| ST070 | wt | −0.20 | 1.2 | 1.1 | 83 |
| ST024 | wt | 0.02 | 1.2 | 2.8 | 46 |
| ST013 | wt | 0.05 | 1.1 | 2.0 | 59 |
| ST004 | KRAS mut | 0.14 | 1.2 | 0.82 | 38 |
| ST022 | KRAS mut | 0.26 | 1.3 | 0.74 | 45 |
| ST040 | ND | ND | ND | ND | 19 |
| ST088 | ND | ND | ND | ND | 88 |
| ST103 | wt | ND | ND | ND | 59 |

Models are listed in order of descending RAS score values.
wt = wildtype;
mut = mutant;
ND = not done

To provide further support for low RAS and high IGF as response biomarkers, the anti-tumor activity of ridaforolimus and dalotuzumab was assessed in 12 molecularly annotated patient-derived primary xenograft (PDX) ovarian cancer models (Table 4). Responses to combination therapy were assessed by percentage tumor growth inhibition (TGI) at the end of 18-28 days of therapy. TGI values ranged from minimal (19% TGI) to significant regression (139% TGI). Gene expression profiling data were generated for nine of the models to correlate response with genotype. More responsive tumor models tended to be associated with a low RAS gene signature and higher expression levels of IGF1 or IGF2. In contrast, tumors with KRAS mutations or a high RAS gene score were generally resistant to combination therapy.

### Example 4

### Evaluation of IGF1 Cq Value in 44 Colorectal Cancer Samples

An independent set of 44 colorectal cancer FFPE tissue specimens (including stromal and tumor cells) was evaluated with the IGF1 real time PCR FFPE assay to determine the relative distribution of IGF1 expression levels and to determine potential cut-points for identifying patients using FFPE specimens as being IGF1 (+) or not. Prior IGF1 evaluation has suggested that IGF1 increased expression is elevated in approximately 18% (+/−5% STDEV) of colorectal cancer specimens above background noise. Based on this, we determined from the set of independent 44 colorectal specimens tested with our specific IGF1 real time PCR FFPE assay that the approximate cut-point for determining (+) IGF1 expression was less than or equal to approximately between 1.83-2.03 (Table 3+/−5% STDEV of 1.93=18% cut-point; n=44) Cq value. Therefore, colorectal cancer samples determined to have IGF1 Cq value< or =2.03 (upper boundary+5% STDEV of 1.93) are likely to be IGF1 (+) and may derive a clinical benefit by treatment with an IGF1R inhibitor.

TABLE 5
Cq values determined for 44 colorectal cancer samples.

| Random Set of | | |
|---|---|---|
| Colorectal | | |
| Cancer Samples | IGF1 RT-PCR | IGF1 Likely |
| (n = 44) | Cq values | Positive (+) |
| 1 | −1.04 | (+) |
| 2 | −0.12 | (+) |
| 3 | 0.25 | (+) |
| 4 | 1.11 | (+) |
| 5 | 1.61 | (+) |
| 6 | 1.63 | (+) |
| 7 | 1.69 | (+) |
| 8 | 1.93 | (+) |
| 9 | 2.14 | |
| 10 | 2.19 | |
| 11 | 2.39 | |
| 12 | 2.4 | |
| 13 | 2.49 | |
| 14 | 2.53 | |
| 15 | 2.75 | |
| 16 | 2.76 | |
| 17 | 2.93 | |
| 18 | 2.94 | |
| 19 | 2.98 | |
| 20 | 3.01 | |
| 21 | 3.09 | |
| 22 | 3.13 | |
| 23 | 3.29 | |
| 24 | 3.48 | |
| 25 | 3.61 | |
| 26 | 3.65 | |
| 27 | 3.66 | |
| 28 | 3.73 | |
| 29 | 3.75 | |
| 30 | 3.86 | |
| 31 | 3.92 | |
| 32 | 4.04 | |
| 33 | 4.3 | |
| 34 | 4.39 | |
| 35 | 4.49 | |
| 36 | 4.75 | |
| 37 | 5.01 | |
| 38 | 5.19 | |
| 39 | 5.21 | |
| 40 | 5.46 | |
| 41 | 5.81 | |
| 42 | 6.26 | |
| 43 | 6.97 | |
| 44 | 7.01 | |

### Example 5

### Evaluation of IGF1 Cq Value in 21 Pancreatic Cancer Samples

Twenty one pancreatic cancer FFPE tissue specimens were evaluated with the IGF1 real time PCR FFPE assay to determine the relative distribution of IGF1 expression levels and to determine potential cut-points for identifying pancreatic patients using FFPE specimens as being IGF1 (+) or not. We determined that pancreatic tumors exhibiting IGF1 expression levels above the median observed in this study were IGF1 (+) whereas expression below the median was IGF1(−). Thus, tumors with an IGF1 Cq value ≦2.87 were considered to be IGF1 high expressers, IGF1(+).

TABLE 6
IGF-1 RT-PCR Data Analysis (n = 21)

| | | | Normal- | | | | | |
|---|---|---|---|---|---|---|---|---|
| | | | ized | | | trt | | |
| | | | IGF1 | Pt | trt | (cy- | PFS | OS |
| | # | SampleName | Cq | ID | arm | cles) | (wks) | (wks) |
| IGF1(+) | 1 | S0748PA0015 | 0.24 | 26 | A | 2 | 8 | 45 |
| | 2 | S0748PA0005 | 0.56 | 8 | A | 12 | 61 | 83 |
| | 3 | S0748PA0032 | 2.34 | 52 | A | 2 | 8 | 15 |
| | 4 | S0748PA0001 | 2.71 | 1 | A | 8 | 62 | 82 |
| | 5 | S0748PA0007 | 1.23 | 11 | B | 2 | 8 | 38 |
| | 6 | S0748PA0030 | 2.51 | 48 | B | 2 | 5 | 23 |
| | 7 | S0748PA0020 | 2.56 | 36 | B | 2 | 8 | 22 |
| | 8 | S0748PA0033 | 2.85 | 54 | B | 2.00 | 8 | 24 |
| | 9 | S0748PA0021 | 0.42 | 38 | C | 2 | 8 | 48 |
| | 10 | S0748PA0031 | 2.47 | 51 | C | 3.50 | 9 | 17 |
| | 11 | S0748PA0027 | 2.87 | 58 | C | 1 | 6 | 9 |

Cut point

| | | | | | | | | |
|---|---|---|---|---|---|---|---|---|
| IGF1(−) | 12 | S0748PA0029 | 3.69 | 45 | A | 2 | 8 | 30 |
| | 13 | S0748PA0006 | 4.26 | 9 | A | 8 | 31 | 48 |
| | 14 | S0748PA0026 | 5.88 | 57 | A | 2 | 10 | 17 |
| | 15 | S0748PA0008 | 6.1 | 16 | A | 2 | 5 | 39 |
| | 16 | S0748PA0004 | 3.55 | 6 | B | 2 | 8 | |
| | | | | | | | alive | |
| | 17 | S0748PA0011 | 5.22 | 19 | B | 2.00 | 9 | 19 |
| | 18 | S0748PA0028 | 5.97 | 70 | B | 5 | 22 | 31 |
| | 19 | S0748PA0012 | 6.93 | 22 | B | 3 | 13 | 13 |
| | 20 | S0748PA0016 | 3.28 | 30 | C | 4 | 8 | 34 |
| | 21 | S0748PA0017 | 3.43 | 31 | C | 6 | 23 | 63 |

Note -
A total of 21 pts had IGF1 RT-PCR data; Cut-point was established at ‘median’ IGF1 Cq value (≦2.87) - this determined which pts to be IGF1 (+) or IGF1 (−) for analysis
Treatment Arm A = MK-0646 + gemcitabine −> treatment arm to take forward for BMx evaluation
Treatment Arm B = MK-0646 + Erlotinib + gemcitabine
Treatment Arm C = Erlotinib + gemcitabine (this was the control arm)

In Table 7(A-B), below, in Treatment Arm A, MK-0646 and gemcitabine in an IGF1 (+) population showed a median progression free survival (PFS) of 8 months (34 weeks). In Treatment Arm C, erlotinib and gemcitabine in an IGF1(+) population, showed a median PFS of 2 months (8 wks). [P-value (2 sided)=0.08; one-sided=0.04]. This trend was confirmed using affimetrix IGF1 RNA array expression profiling data in part B of Table 7.

TABLE 7
Clinical Results For 21 pts With RT-PCR data
Vs. The 17 pts With Affy FFPE Profiling Data.
(A) 21 pts With qRT-PCR Data

| | Trt | | Partial | # | PFS | OS |
|---|---|---|---|---|---|---|
| Biomarker | Arm | N = 21 | Responders | cycles | (wks) | (wks) |
| IGF1 (+) | A | 4 | 2 | 6.0 | 34.5 | 63.5 |
| | B | 4 | 0 | 2.0 | 8.0 | 23.5 |
| | C | 3 | 0 | 2.2 | 8.0 | 17.0 |
| IGF1 (−) | A | 4 | 1 | 3.5 | 9.0 | 34.5 |
| | B | 4 | 0 | 3.0 | 11.0 | 19.0 |
| | C | 2 | 1 | 5.0 | 15.5 | 48.5 |

(B) 17 pts For which we have Affy Profiling Data
(4 patients lost during the profiling attempt).

| | Trt | | Partial | # | PFS | OS |
|---|---|---|---|---|---|---|
| Biomarker | Arm | N = 17 | Responders | cycles | (wks) | (wks) |
| IGF1 (+) | A | 4 | 2 | 6.0 | 34.5 | 63.5 |
| | B | 3 | 0 | 2.0 | 8.0 | 23.0 |
| | C | 2 | 0 | 1.5 | 7.0 | 28.5 |
| IGF1 (−) | A | 4 | 1 | 3.5 | 9.0 | 34.5 |
| | B | 2 | 0 | 2.5 | 10.5 | 13.0 |
| | C | 2 | 1 | 5.0 | 15.5 | 48.5 |

Note -
the IGF1 (+/−) status above was detemined by ‘qRT-PCR’ in both sets. Overall, we observed similar clinical results between the two sample data sets.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

1. A method for treating a tumor in a subject in need of such treatment, that expresses IGF1 mRNA, comprising determining if the fractional cycle number of a real time polymerase chain reaction amplification of IGF1 cDNA, that was reverse transcribed from IGF1 mRNA from a cell of said tumor, normalized relative to that of one or more reference genes, wherein acceleration of amplification is at a maximum, is at or below about 2.03 or at or below about 2.87; and, if the fractional cycle number is at or below about 2.03 or at or below about 2.87, then administering a therapeutically effective amount of an IGF1R inhibitor, optionally in association with gemcitabine and/or ridaforolimus, to said subject.

2. The method of claim 1 wherein the point at which acceleration of amplification is at a maximum is determined by determining the second derivative maxima of an amplification curve of the reaction.

3. The method of claim 1 wherein the level of IGF1 mRNA expression in cells of said tumor is determined by a method comprising:
(a) obtaining cells of the subject's tumor;
(b) isolating RNA from said cells
(c) generating cDNA by reverse transcribing the RNA
(d) separately amplifying the cDNA encoding IGF1 and encoding one or more reference genes selected from the group consisting of: CYC1, HMBS, TOP1, SDHA, GUSB, PUM1, HPRT1, ACTB, UBC, B2M, GAPDH, and TUBB2A while monitoring production of the cDNA during the amplification; and
(e) determining the quantity amplified cDNA generated and;
optionally, normalizing the determined quantity of IGF1 with that of the reference gene(s).

4. The method of claim 1 wherein the IGF1R inhibitor is a member selected from the group consisting of dalotuzumab, robatumumab, figitumumab, cixutumumab, ganitumab, AVE1642, OSI-906, NVP-AEW541 and NVP-ADW742.

5. The method of claim 1 wherein, if the subject is administered the IGF1R inhibitor, then the subject is also administered a further chemotherapeutic agent.

6. The method of claim 5 wherein the further chemotherapeutic agent is a member selected from the group consisting of:
5-fluorouridine; 131-I-TM-601; 13-cis-retinoic acid; 3-[5-(methylsulfonylpiperadinemethyl)-indolyl]-quinolone; 40-O-(2-hydroxyethyl)-rapamycin; 4-hydroxytamoxifen; 5-deooxyuridine; 6-mercaptopurine; 7-hydroxy staurosporine; a combination of irinotecan, 5-fluorouracil and leucovorin; a combination of oxaliplatin fluorouracil and folinic acid; A-443654; abiraterone acetate; abraxane; ABT-578; acolbifene; ADS-100380; AG-013736; alprazolam; ALT-110; altretamine; amifostine; aminoglutethimide; AMN-107; amrubicin; amsacrine; an antiandrogen; anagrelide; anastrazole; angiostatin; an EGF Receptor antagonuist; an selective estrogen receptor modulator (SERM); an AKT inhibitor; an anti-angiogenesis agents; an anti-EGFR antibody; an anti-emetic; an anti-HER2 antibody; an anti-VEGF antibody; an aromatase inhibitor; an CDK inhibitor; an CYP17 lyase inhibitor; an estrogen; an GnRH agonists; a HER2 antagonist; a lutenizing hormone-releasing hormone agonist; an MEK inhibitor; an mTOR inhibitor; an NK-1 receptor antagonists; a PI3 kinase inhibitor; a progestational agent; a Raf inhibitor; a VEGFR inhibitor; AP-23573; aprepitant; ARQ-197; arzoxifene; AS-252424; AS-605240; asparaginase; AT-9263; atrasentan; AV-299; AZD 1152; AZD 6244; azd2171; AZD-6244; Bacillus Calmette-Guerin vaccine; batabulin; BC-210; bevacizumab; bicalutamide; Bio 111; BIO 140; bleomycin; BMS-214662; BMS-247550; BMS-275291; BMS-310705; bortezimib; buserelin; busulfan; calcitriol; camptothecin; canertinib; capecitabine; carboplatin; carmustine; casopitant; CC 8490; CG-1521; CG-781; chlamydocin; chlorambucil; cilengitide; cimitidine; cisplatin; Cladribine; clodronate; COL-3; conjugated estrogens; CP-724714; cyclophosphamide; cyproterone; cyproterone acetate; cytarabine; cytosine arabinoside; cytproterone acetate; dacarbazine; dactinomycin; darbepoetin alfa; dasatanib; daunorubicin; decatanib; deguelin; denileukin; deoxycoformycin; depsipeptide; DES(diethylstilbestrol); dexamethasone; diarylpropionitrile; diethylstilbestrol; diftitox; diphenhydramine; DN-101; docetaxel; dolasetron; dovitinib; doxorubicin; doxorubicin HCl liposome injection; droloxifene; dronabinol; droperidol; edotecarin; edotreotide (yttrium-90 labeled or unlabeled); EKB-569; EMD121974; endostatin; enzastaurin; epirubicin; epithilone B; epoetin alfa; ERA-923; erbitux; erlotinib; erythropoietin; estradiol; estramustine; etoposide; everolimus; exemestane; finasteride; flavopiridol; floxuridine; fludarabine; fludrocortisones; fluoxymesterone; flutamide; fulvestrant; galeterone; GDC-0941; gefitinib; gemcitabine; a combination of gemcitabine in association with erlotinib; gimatecan; goserelin; goserelin acetate; gossypol; granisetron; GSK461364; GSK690693; GW-572016; haloperidol; HKI-272; HMR-3339; hydroxyprogesterone caproate; hydroxyurea; hydroxyzine; IC87114; idarubicin; Idoxifene; ifosfamide; IL13-PE38QQR; IM862; imatinib; IMC-1C11; INO 1001; interferon; interleukin-12; IPdR; ipilimumab; irinotecan; JNJ-16241199; ketoconazole; KRN951; KRX-0402; L-779,450; lapatanib; lasofoxifene; Lep-etu; letrozole; leucovorin; leuprolide; leuprolide acetate; levamisole; lomustine; lonafarnib; lorazepam; Lucanthone; LY 317615; LY292223; LY292696; LY293646; LY293684; LY294002; marimastat; MDV-3100; mechlorethamine; medroxyprogesterone acetate; megestrol acetate; melphalan; mercaptopurine; mesna; methotrexate; methylprednisolone; metoclopramide; mithramycin; mitomycin; mitotane; mitoxantrone; MK-0457; MLN8054; neovastat; netupitant; neuradiab; nilotinib; nilutimide; nolatrexed; NVP-BEZ235; NVP-LAQ824; oblimersen; octreotide; ofatumumab; ON 0910.Na; ondansetron; oregovomab; orteronel; oxaliplatin; paclitaxel; palonosetron; pamidronate; panitumumab; pazopanib; PD0325901; PEG-filgrastim; PEG-interferon; PEG-labeled irinotecan; pemetrexed; pentostatin; perifosine; PHA-739358; phenylalanine mustard; PI-103; PIK-75; pipendoxifene; PKI-166; plicamycin; porfimer; prednisone; procarbazine; prochlorperazine; progestins; PTK787/ZK 222584; PX-866; R-763; RAD001; raloxifene; raltitrexed; razoxin; ridaforolimus; rituximab; romidepsin; RTA 744; rubitecan; scriptaid; Sdx 102; seliciclib; semaxanib; SF1126; sirolimus; SN36093; sorafenib; spironolactone; squalamine; SR13668; streptozocin; SU6668; suberoyl analide hydroxamic acid; sunitinib; sunitinib malate; talampanel; tamoxifen; temozolomide; temsirolimus; teniposide; tesmilifene; testosterone; tetrandrine; TGX-221; thalidomide; thioguanine; thiotepa; ticilimumab; tipifarnib; TKI-258; TLK 286; topotecan; toremifene citrate; trabectedin; trastuzumab; tretinoin; trichostatin A; triciribine phosphate monohydrate; triptorelin pamoate; tropisetron; TSE-424; uracil mustard; valproic acid; valrubicin; vandetanib; vatalanib; VEGF trap; vinblastine; vincristine; vindesine; vinorelbine; vitaxin; vitespan; vorinostat; VX-745; wortmannin; Xr 311; zanolimumab; ZK186619; ZK-304709, ZM336372; ZSTK474; cis-3-[4-[(4-chlorophenyl)sulfonyl]-4-(2,5-difluorophenyl)cyclohexyl]propanoic acid;

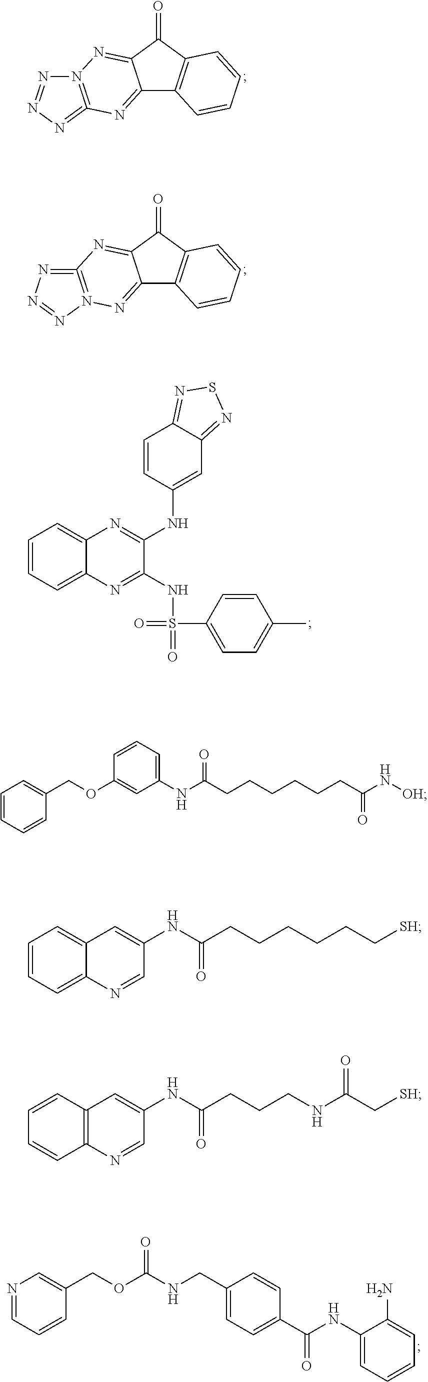

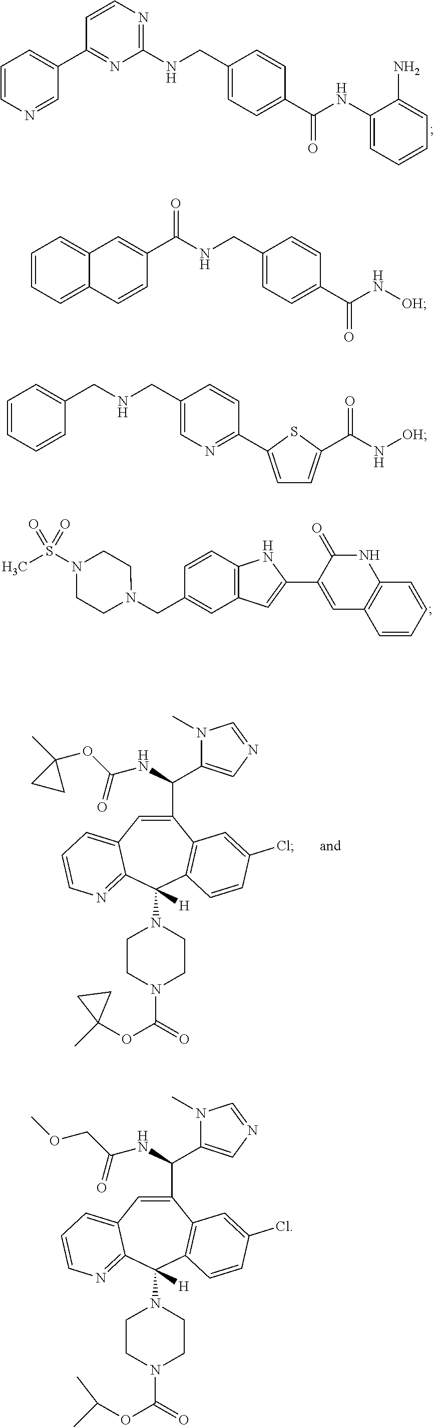

7. A method for selecting a subject with a tumor for treatment with an IGF1R inhibitor, optionally in association with gemcitabine and/or ridaforolimus, comprising determining if the fractional cycle number of a real time polymerase chain reaction amplification of IGF1 cDNA, that was reverse transcribed from IGF1 mRNA from a cell of said tumor, normalized relative to that of one or more reference genes, in which acceleration of amplification is at a maximum, is at or below about 2.03 or at or below about 2.87 and, if the fractional cycle number is at or below about 2.03 or at or below about 2.87, then selecting the subject for treatment of the tumor with the IGF1R inhibitor, optionally in association with gemcitabine and/or ridaforolimus; and, optionally, further comprising administering a therapeutically effective amount of IGF1R inhibitor, optionally in association with gemcitabine and/or ridaforolimus, to the subject.

8. The method of claim 7 wherein the point at which acceleration of amplification is at a maximum is determined by determining the second derivative maxima of an amplification curve of the reaction.

9. The method of claim 7 further comprising administering a therapeutically effective amount of IGF1R inhibitor to the subject.

10. The method of claim 7 wherein the IGF1R inhibitor is a member selected from the group consisting of dalotuzumab, robatumumab, figitumumab, cixutumumab, ganitumab, AVE1642, OSI-906, NVP-AEW541 and NVP-ADW742.

11. The method of claim 7 wherein, if the subject is administered the IGF1R inhibitor, then the subject is also administered a further chemotherapeutic agent.

12. The method of claim 11 wherein the further chemotherapeutic agent is a member selected from the group consisting of:
5-fluorouridine; 131-I-TM-601; 13-cis-retinoic acid; 3-[5-(methylsulfonylpiperadinemethyl)-indolyl]-quinolone; 40-O-(2-hydroxyethyl)-rapamycin; 4-hydroxytamoxifen; 5-deooxyuridine; 6-mercaptopurine; 7-hydroxy staurosporine; a combination of irinotecan, 5-fluorouracil and leucovorin; a combination of oxaliplatin fluorouracil and folinic acid; A-443654; abiraterone acetate; abraxane; ABT-578; acolbifene; ADS-100380; AG-013736; alprazolam; ALT-110; altretamine; amifostine; aminoglutethimide; AMN-107; amrubicin; amsacrine; an antiandrogen; anagrelide; anastrazole; angiostatin; an EGF Receptor antagonuist; an selective estrogen receptor modulator (SERM); an AKT inhibitor; an anti-angiogenesis agents; an anti-EGFR antibody; an anti-emetic; an anti-HER2 antibody; an anti-VEGF antibody; an aromatase inhibitor; an CDK inhibitor; an CYP17 lyase inhibitor; an estrogen; an GnRH agonists; a HER2 antagonist; a lutenizing hormone-releasing hormone agonist; an MEK inhibitor; an mTOR inhibitor; an NK-1 receptor antagonists; a PI3 kinase inhibitor; a progestational agent; a Raf inhibitor; a VEGFR inhibitor; AP-23573; aprepitant; ARQ-197; arzoxifene; AS-252424; AS-605240; asparaginase; AT-9263; atrasentan; AV-299; AZD 1152; AZD 6244; azd2171; AZD-6244; Bacillus Calmette-Guerin vaccine; batabulin; BC-210; bevacizumab; bicalutamide; Bio 111; BIO 140; bleomycin; BMS-214662; BMS-247550; BMS-275291; BMS-310705; bortezimib; buserelin; busulfan; calcitriol; camptothecin; canertinib; capecitabine; carboplatin; carmustine; casopitant; CC 8490; CG-1521; CG-781; chlamydocin; chlorambucil; cilengitide; cimitidine; cisplatin; Cladribine; clodronate; COL-3; conjugated estrogens; CP-724714; cyclophosphamide; cyproterone; cyproterone acetate; cytarabine; cytosine arabinoside; cytproterone acetate; dacarbazine; dactinomycin; darbepoetin alfa; dasatanib; daunorubicin; decatanib; deguelin; denileukin; deoxycoformycin; depsipeptide; DES(diethylstilbestrol); dexamethasone; diarylpropionitrile; diethylstilbestrol; diftitox; diphenhydramine; DN-101; docetaxel; dolasetron; dovitinib; doxorubicin; doxorubicin HCl liposome injection; droloxifene; dronabinol; droperidol; edotecarin; edotreotide (yttrium-90 labeled or unlabeled); EKB-569; EMD121974; endostatin; enzastaurin; epirubicin; epithilone B; epoetin alfa; ERA-923; erbitux; erlotinib; erythropoietin; estradiol; estramustine; etoposide; everolimus; exemestane; finasteride; flavopiridol; floxuridine; fludarabine; fludrocortisones; fluoxymesterone; flutamide; fulvestrant; galeterone; GDC-0941; gefitinib; gemcitabine; a combination of gemcitabine in association with erlotinib; gimatecan; goserelin; goserelin acetate; gossypol; granisetron; GSK461364; GSK690693; GW-572016; haloperidol; HKI-272; HMR-3339; hydroxyprogesterone caproate; hydroxyurea; hydroxyzine; IC87114; idarubicin; Idoxifene; ifosfamide; IL13-PE38QQR; IM862; imatinib; IMC-1C11; INO 1001; interferon; interleukin-12; IPdR; ipilimumab; irinotecan; JNJ-16241199; ketoconazole; KRN951; KRX-0402; L-779,450; lapatanib; lasofoxifene; Lep-etu; letrozole; leucovorin; leuprolide; leuprolide acetate; levamisole; lomustine; lonafarnib; lorazepam; Lucanthone; LY 317615; LY292223; LY292696; LY293646; LY293684; LY294002; marimastat; MDV-3100; mechlorethamine; medroxyprogesterone acetate; megestrol acetate; melphalan; mercaptopurine; mesna; methotrexate; methylprednisolone; metoclopramide; mithramycin; mitomycin; mitotane; mitoxantrone; MK-0457; MLN8054; neovastat; netupitant; neuradiab; nilotinib; nilutimide; nolatrexed; NVP-BEZ235; NVP-LAQ824; oblimersen; octreotide; ofatumumab; ON 0910.Na; ondansetron; oregovomab; orteronel; oxaliplatin; paclitaxel; palonosetron; pamidronate; panitumumab; pazopanib; PD0325901; PEG-filgrastim; PEG-interferon; PEG-labeled irinotecan; pemetrexed; pentostatin; perifosine; PHA-739358; phenylalanine mustard; PI-103; PIK-75; pipendoxifene; PKI-166; plicamycin; porfimer; prednisone; procarbazine; prochlorperazine; progestins; PTK787/ZK 222584; PX-866; R-763; RAD001; raloxifene; raltitrexed; razoxin; ridaforolimus; rituximab; romidepsin; RTA 744; rubitecan; scriptaid; Sdx 102; seliciclib; semaxanib; SF1126; sirolimus; SN36093; sorafenib; spironolactone; squalamine; SR13668; streptozocin; SU6668; suberoyl analide hydroxamic acid; sunitinib; sunitinib malate; talampanel; tamoxifen; temozolomide; temsirolimus; teniposide; tesmilifene; testosterone; tetrandrine; TGX-221; thalidomide; thioguanine; thiotepa; ticilimumab; tipifarnib; TKI-258; TLK 286; topotecan; toremifene citrate; trabectedin; trastuzumab; tretinoin; trichostatin A; triciribine phosphate monohydrate; triptorelin pamoate; tropisetron; TSE-424; uracil mustard; valproic acid; valrubicin; vandetanib; vatalanib; VEGF trap; vinblastine; vincristine; vindesine; vinorelbine; vitaxin; vitespan; vorinostat; VX-745; wortmannin; Xr 311; zanolimumab; ZK186619; ZK-304709, ZM336372; ZSTK474; cis-3-[4-[(4-chlorophenyl)sulfonyl]-4-(2,5-difluorophenyl)cyclohexyl]propanoic acid;

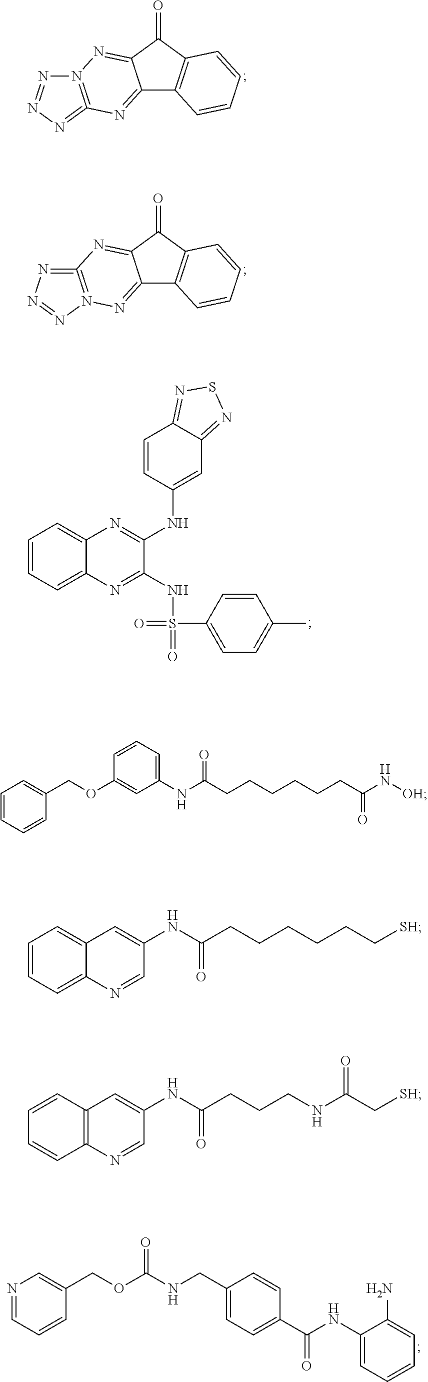

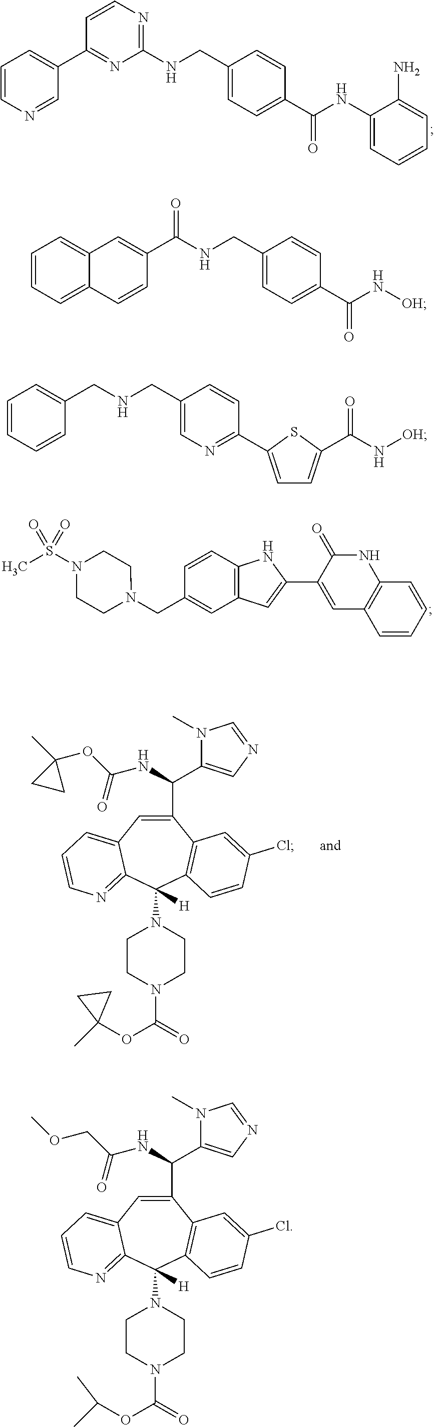

13. A method for selecting a therapy for a subject with a tumor comprising determining if the fractional cycle number of a real time polymerase chain reaction amplification of IGF1 cDNA, that was reverse transcribed from IGF1 mRNA from a cell of said tumor, normalized relative to that of one or more reference genes, in which acceleration of amplification is at a maximum, is at or below about 2.03 or at or below about 2.87; and if the fractional cycle number is at or below about 2.03 or at or below about 2.87; then selecting an IGF1R inhibitor, optionally in association with gemcitabine and/or ridaforolimus, for treatment of the tumor in the subject; and, optionally, further comprising administering a therapeutically effective amount of IGF1R inhibitor, optionally in association with gemcitabine and/or ridaforolimus, to the subject.

14. The method of claim 13 wherein the point at which acceleration of amplification is at a maximum is determined by determining the second derivative maxima of an amplification curve of the reaction.

15. The method claim 13 further comprising administering a therapeutically effective amount of IGF1R inhibitor to the subject.

16. The method of claim 13 wherein the IGF1R inhibitor is a member selected from the group consisting of dalotuzumab, robatumumab, figitumumab, cixutumumab, ganitumab, AVE1642, OSI-906, NVP-AEW541 and NVP-ADW742.

17. The method of claim 13 wherein, if the subject is administered a therapeutically effective amount of IGF1R inhibitor then the subject is also administered a further chemotherapeutic agent.

18. The method of claim 17 wherein the further chemotherapeutic agent is a member selected from the group consisting of:
5-fluorouridine; 131-I-TM-601; 13-cis-retinoic acid; 3-[5-(methylsulfonylpiperadinemethyl)-indolyl]-quinolone; 40-O-(2-hydroxyethyl)-rapamycin; 4-hydroxytamoxifen; 5-deooxyuridine; 6-mercaptopurine; 7-hydroxy staurosporine; a combination of irinotecan, 5-fluorouracil and leucovorin; a combination of oxaliplatin fluorouracil and folinic acid; A-443654; abiraterone acetate; abraxane; ABT-578; acolbifene; ADS-100380; AG-013736; alprazolam; ALT-110; altretamine; amifostine; aminoglutethimide; AMN-107; amrubicin; amsacrine; an antiandrogen; anagrelide; anastrazole; angiostatin; an EGF Receptor antagonuist; an selective estrogen receptor modulator (SERM); an AKT inhibitor; an anti-angiogenesis agents; an anti-EGFR antibody; an anti-emetic; an anti-HER2 antibody; an anti-VEGF antibody; an aromatase inhibitor; an CDK inhibitor; an CYP17 lyase inhibitor; an estrogen; an GnRH agonists; a HER2 antagonist; a lutenizing hormone-releasing hormone agonist; an MEK inhibitor; an mTOR inhibitor; an NK-1 receptor antagonists; a PI3 kinase inhibitor; a progestational agent; a Raf inhibitor; a VEGFR inhibitor; AP-23573; aprepitant; ARQ-197; arzoxifene; AS-252424; AS-605240; asparaginase; AT-9263; atrasentan; AV-299; AZD 1152; AZD 6244; azd2171; AZD-6244; Bacillus Calmette-Guerin vaccine; batabulin; BC-210; bevacizumab; bicalutamide; Bio 111; BIO 140; bleomycin; BMS-214662; BMS-247550; BMS-275291; BMS-310705; bortezimib; buserelin; busulfan; calcitriol; camptothecin; canertinib; capecitabine; carboplatin; carmustine; casopitant; CC 8490; CG-1521; CG-781; chlamydocin; chlorambucil; cilengitide; cimitidine; cisplatin; Cladribine; clodronate; COL-3; conjugated estrogens; CP-724714; cyclophosphamide; cyproterone; cyproterone acetate; cytarabine; cytosine arabinoside; cytproterone acetate; dacarbazine; dactinomycin; darbepoetin alfa; dasatanib; daunorubicin; decatanib; deguelin; denileukin; deoxycoformycin; depsipeptide; DES(diethylstilbestrol); dexamethasone; diarylpropionitrile; diethylstilbestrol; diftitox; diphenhydramine; DN-101; docetaxel; dolasetron; dovitinib; doxorubicin; doxorubicin HCl liposome injection; droloxifene; dronabinol; droperidol; edotecarin; edotreotide (yttrium-90 labeled or unlabeled); EKB-569; EMD121974; endostatin; enzastaurin; epirubicin; epithilone B; epoetin alfa; ERA-923; erbitux; erlotinib; erythropoietin; estradiol; estramustine; etoposide; everolimus; exemestane; finasteride; flavopiridol; floxuridine; fludarabine; fludrocortisones; fluoxymesterone; flutamide; fulvestrant; galeterone; GDC-0941; gefitinib; gemcitabine; a combination of gemcitabine in association with erlotinib; gimatecan; goserelin; goserelin acetate; gossypol; granisetron; GSK461364; GSK690693; GW-572016; haloperidol; HKI-272; HMR-3339; hydroxyprogesterone caproate; hydroxyurea; hydroxyzine; IC87114; idarubicin; Idoxifene; ifosfamide; IL13-PE38QQR; IM862; imatinib; IMC-1C11; INO 1001; interferon; interleukin-12; IPdR; ipilimumab; irinotecan; JNJ-16241199; ketoconazole; KRN951; KRX-0402; L-779,450; lapatanib; lasofoxifene; Lep-etu; letrozole; leucovorin; leuprolide; leuprolide acetate; levamisole; lomustine; lonafarnib; lorazepam; Lucanthone; LY 317615; LY292223; LY292696; LY293646; LY293684; LY294002; marimastat; MDV-3100; mechlorethamine; medroxyprogesterone acetate; megestrol acetate; melphalan; mercaptopurine; mesna; methotrexate; methylprednisolone; metoclopramide; mithramycin; mitomycin; mitotane; mitoxantrone; MK-0457; MLN8054; neovastat; netupitant; neuradiab; nilotinib; nilutimide; nolatrexed; NVP-BEZ235; NVP-LAQ824; oblimersen; octreotide; ofatumumab; ON 0910.Na; ondansetron; oregovomab; orteronel; oxaliplatin; paclitaxel; palonosetron; pamidronate; panitumumab; pazopanib; PD0325901; PEG-filgrastim; PEG-interferon; PEG-labeled irinotecan; pemetrexed; pentostatin; perifosine; PHA-739358; phenylalanine mustard; PI-103; PIK-75; pipendoxifene; PKI-166; plicamycin; porfimer; prednisone; procarbazine; prochlorperazine; progestins; PTK787/ZK 222584; PX-866; R-763; RAD001; raloxifene; raltitrexed; razoxin; ridaforolimus; rituximab; romidepsin; RTA 744; rubitecan; scriptaid; Sdx 102; seliciclib; semaxanib; SF1126; sirolimus; SN36093; sorafenib; spironolactone; squalamine; SR13668; streptozocin; SU6668; suberoyl analide hydroxamic acid; sunitinib; sunitinib malate; talampanel; tamoxifen; temozolomide; temsirolimus; teniposide; tesmilifene; testosterone; tetrandrine; TGX-221; thalidomide; thioguanine; thiotepa; ticilimumab; tipifarnib; TKI-258; TLK 286; topotecan; toremifene citrate; trabectedin; trastuzumab; tretinoin; trichostatin A; triciribine phosphate monohydrate; triptorelin pamoate; tropisetron; TSE-424; uracil mustard; valproic acid; valrubicin; vandetanib; vatalanib; VEGF trap; vinblastine; vincristine; vindesine; vinorelbine; vitaxin; vitespan; vorinostat; VX-745; wortmannin; Xr 311; zanolimumab; ZK186619; ZK-304709, ZM336372; ZSTK474; cis-3-[4-[(4-chlorophenyl)sulfonyl]-4-(2,5-difluorophenyl)cyclohexyl]propanoic acid;

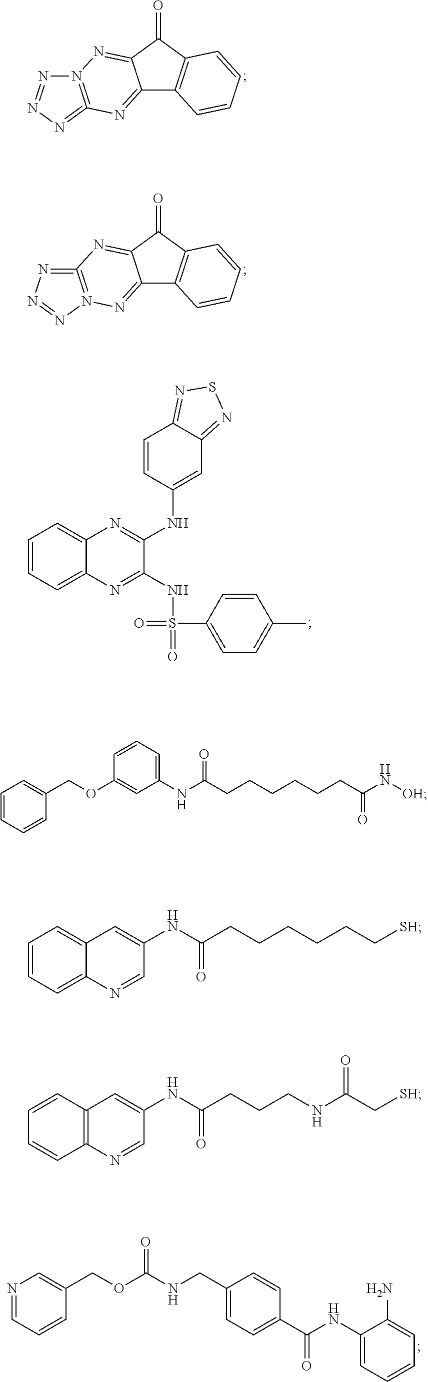

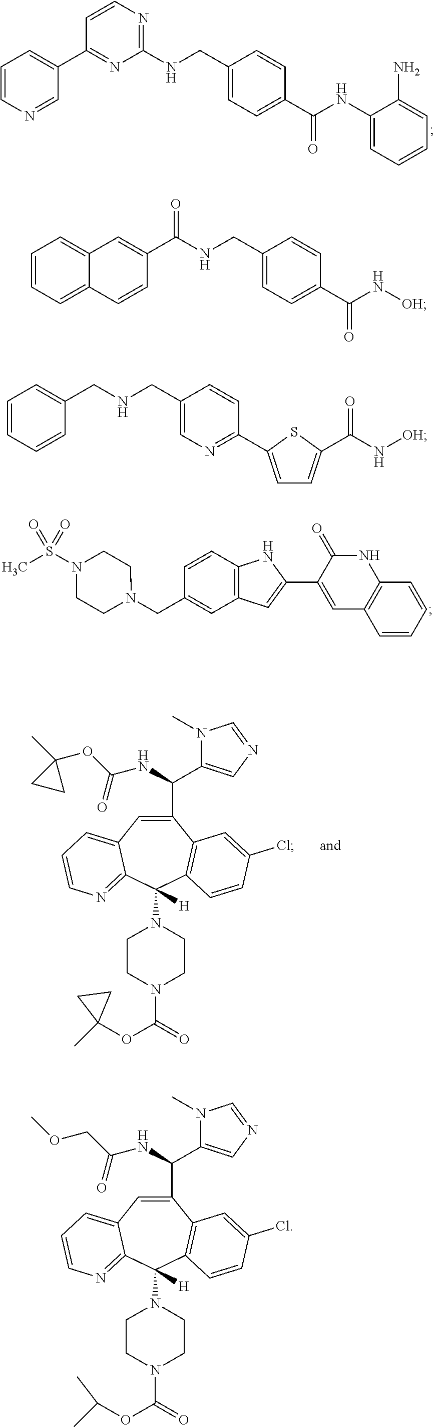

19. A method for evaluating the sensitivity of in vitro tumor cells, obtained from a subject, to IGF1R inhibitor therapy, optionally in association with gemcitabine and/or ridaforolimus, comprising determining if the fractional cycle number of a real time polymerase chain reaction amplification of IGF1 cDNA, that was reverse transcribed from IGF1 mRNA from a cell of said tumor, normalized relative to that of one or more reference genes, in which acceleration of amplification is at a maximum, is at or below about 2.03 or at or below about 2.87; and, if the fractional cycle number is at or below about 2.03 or at or below about 2.87, then determining that the tumor cells are sensitive; and, optionally, further comprising administering a therapeutically effective amount of the IGF1R inhibitor, optionally in association with gemcitabine and/or ridaforolimus, to the subject.

20. The method of claim 19 wherein the point at which acceleration of amplification is at a maximum is determined by determining the second derivative maxima of an amplification curve of the reaction.

21. The method of claim 1 wherein the level of KRAS expression and/or whether KRAS is wild-type or mutated, in the tumor cells, is determined.

22. A kit for determining whether tumor cells are sensitive to an IGF1R inhibitor comprising an IGF1R inhibitor an instructions for performing the method of any claim 19.

23. The method of claim 1 wherein IGF1 comprises the nucleotide sequence set forth in SEQ ID NO: 13 or a cDNA sequence thereof or a nucleotide sequence comprising at least 80% identity thereto and/or wherein the reference gene comprises a nucleotide sequence selected from the group consisting of SEQ ID NOs: 14-25 or a cDNA sequence thereof or a nucleotide sequence comprising at least 80% identity thereto.
